SEQUENCE LISTING (120 sequences)

SEQ ID NO: 1 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 153 aa, Homo sapiens
SENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 3 - protein, 160 aa, Artificial Sequence
  source: /note=“Description of Artificial Sequence Synthetic polypeptide”
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 4 - protein, 213 aa, Artificial Sequence
  source: /note=“Description of Artificial Sequence Synthetic polypeptide”
GTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 5 - protein, 240 aa, Artificial Sequence
  source: /note=“Description of Artificial Sequence Synthetic polypeptide”
DIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 6 - protein, 240 aa, Artificial Sequence
  source: /note=“Description of Artificial Sequence Synthetic polypeptide”
QVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELK

SEQ ID NO: 7 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 8 - protein, 111 aa, Artificial Sequence
  source: /note=“Description of Artificial Sequence Synthetic polypeptide”
DIQMTQSPSSLSASVGDRVTITCRASESVDFYGNSFMHWYQQKPGKAPKLLIYLASNLESGVPSRFSGSGSGTDFTLTISSLQPEDFATYYCQQNIEDPLTFGGGTKVEIK

SEQ ID NO: 9 - protein, 121 aa, Artificial Sequence
  source: /note=“Description of Artificial Sequence Synthetic polypeptide”
QVQLVQSGAEVKKPGASVKVSCKASGYTFTSEWMNWVRQAPGQGLEWMGQIFPALGSTNYNEMYEGRVTMTTDTSTSTAYMELRSLRSDDTAVYYCARGIGNYALDAMDYWGQGTLVTVSS

SEQ ID NO: 10 - protein, 137 aa, Artificial Sequence
  source: /note=“Description of Artificial Sequence Synthetic polypeptide”
GIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 11 - protein, 237 aa, Artificial Sequence
  source: /note=“Description of Artificial Sequence Synthetic polypeptide”
MAWMMLLLGLLAYGSGVDSDIVLTQSPASLAVSLGQRATISCRASLSVSSSGYSHMHWYQQKPGQPPKFLIYLASKLQSGVPARFSGSGSGTDFTLSIHPVEEEDAATYYCQHSRELPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 467 aa, Artificial Sequence
  source: /note=“Description of Artificial Sequence Synthetic polypeptide”
MAWMMLLLGLLAYGSGVDSDVQLVESGGGLVQPGGSRKLSCAASGFTFSTFGMHWVRQTPEKGLDWVAYISSGSTTIHYADSVKGRFTISRDNPKNTLFLEMTSLRSEDTAMYYCSRRMERNGMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 13 - protein, 112 aa, Artificial Sequence [sequence omitted]

SEQ ID NO: 14 - protein, 125 aa, Artificial Sequence [sequence omitted]

SEQ ID NO: 15 - protein, 107 aa, Artificial Sequence [sequence omitted]

SEQ ID NO: 16 - protein, 121 aa, Artificial Sequence [sequence omitted]

SEQ ID NO: 17 - protein, 107 aa, Artificial Sequence [sequence omitted]

SEQ ID NO: 18 - protein, 119 aa, Artificial Sequence [sequence omitted]

SEQ ID NO: 19 - protein, 107 aa, Artificial Sequence
  source: /note=“Description of Artificial Sequence Synthetic polypeptide”
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIK

SEQ ID NO: 20 - protein, 116 aa, Artificial Sequence
  source: /note=“Description of Artificial Sequence Synthetic polypeptide”
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 21 - protein, 111 aa, Artificial Sequence
  source: /note=“Description of Artificial Sequence Synthetic polypeptide”
DIVLTQSPDSLAVSLGERATINCRASESVDTFDYSFLHWYQQKPGQPPKLLIYRASNLESGVPDRFSGSGSGTDFTLTISSLQAEDVAVYYCQQSNEDPYTFGQGTKLEIK

SEQ ID NO: 22 - protein, 121 aa, Artificial Sequence
  source: /note=“Description of Artificial Sequence Synthetic polypeptide”
EVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSS

SEQ ID NO: 23 - protein, 107 aa, Artificial Sequence
  source: /note=“Description of Artificial Sequence Synthetic polypeptide”
DIQMTQSPASISASVGETVTITCRASENIYSYLVWYQQKEGKTPHLLVYNAKTLAEGVPSRFSGSGSGTQFSLKINSLQPEDFGTYYCQHHYGTPLTFGAGTKLELK

SEQ ID NO: 24 - protein, 122 aa, Artificial Sequence
  source: /note=“Description of Artificial Sequence Synthetic polypeptide”
QVQLQQSGAELARPGASVKLSCKASGFTFTDHFINWVRQRTGQGLEWIGEIYPGTGNAFYSEKFKGKATLTADKSSSTAYMHLSSLTSEDSAVFFCASPLRSGSHYWYFDVWGAGTTVTVSS

SEQ ID NO: 25 - protein, 150 aa, Artificial Sequence
  source: /note=“Description of Artificial Sequence Synthetic polypeptide”
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 26 - protein, 219 aa, Artificial Sequence
  source: /note=“Description of Artificial Sequence Synthetic polypeptide”
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 627 aa, Artificial Sequence
  source: /note=“Description of Artificial Sequence Synthetic polypeptide”
  SITE (453)..(467): /note=“This region may encompass 0-3 ′Gly Gly Gly Gly Ser′ repeating units”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 28 - protein, 833 aa, Artificial Sequence
  source: /note=“Description of Artificial Sequence Synthetic polypeptide”
  SITE (453)..(467): /note=“This region may encompass 0-3 ′Gly Gly Gly Gly Ser′ repeating units”
  SITE (628)..(662): /note=“This region may encompass 1-5 ′Gly Gly Gly Gly Ser Ala Ala′ repeating units”
  SITE (663)..(673): /note=“This region may or may not be present”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAAPYAYWMRGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 29 - protein, 1019 aa, Artificial Sequence
  source: /note=“Description of Artificial Sequence Synthetic polypeptide”
  SITE (453)..(487): /note=“This region may encompass 1-5 ′Gly Gly Gly Gly Ser Ala Ala′ repeating units”
  SITE (728)..(762): /note=“This region may encompass 1-5 ′Gly Gly Gly Gly Ser Ala Ala′ repeating units”
  SITE (763)..(769): /note=“This region may or may not be present”
  SITE (770)..(779): /note=“This region may encompass 0-2 ′Gly Gly Gly Gly Ser′ repeating units”
  SITE (728)..(1019): /note=“This region may or may not be present”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSAAGGGGSAAGGGGSAAGGGGSAAGGGGSAADIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSAAGGGGSAAGGGGSAAGGGGSAAGGGGSAAPYAYWMRGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 30 - protein, 1019 aa, Artificial Sequence
  source: /note=“Description of Artificial Sequence Synthetic polypeptide”
  SITE (453)..(487): /note=“This region may encompass 1-5 ′Gly Gly Gly Gly Ser Ala Ala′ repeating units”
  SITE (728)..(762): /note=“This region may encompass 1-5 ′Gly Gly Gly Gly Ser Ala Ala′ repeating units”
  SITE (763)..(769): /note=“This region may or may not be present”
  SITE (770)..(779): /note=“This region may encompass 0-2 ′Gly Gly Gly Gly Ser′ repeating units”
  SITE (728)..(1019): /note=“This region may or may not be present”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSAAGGGGSAAGGGGSAAGGGGSAAGGGGSAAQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSAAGGGGSAAGGGGSAAGGGGSAAGGGGSAAPYAYWMRGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELK

SEQ ID NO: 31 - protein, 939 aa, Artificial Sequence
  source: /note=“Description of Artificial Sequence Synthetic polypeptide”
  SITE (453)..(487): /note=“This region may encompass 0-5 ′Gly Gly Gly Gly Ser Ala Ala′ repeating units”
  SITE (648)..(682): /note=“This region may encompass 0-5 ′Gly Gly Gly Gly Ser Ala Ala′ repeating units”
  SITE (683)..(689): /note=“This region may or may not be present”
  SITE (690)..(699): /note=“This region may encompass 0-2 ′Gly Gly Gly Gly Ser′ repeating units”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAAPYAYWMRGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 32 - protein, 923 aa, Artificial Sequence
  source: /note=“Description of Artificial Sequence Synthetic polypeptide”
  SITE (453)..(477): /note=“This region may encompass 0-5 ′Gly Gly Gly Gly Ser′ repeating units”
  SITE (718)..(752): /note=“This region may encompass 0-5 ′Gly Gly Gly Gly Ser Ala Ala′ repeating units”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSAAGGGGSAAGGGGSAAGGGGSAAGGGGSAAPYAYWMRGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 33 - protein, 939 aa, Artificial Sequence
  source: /note=“Description of Artificial Sequence Synthetic polypeptide”
  SITE (453)..(487): /note=“This region may encompass 0-5 ′Gly Gly Gly Gly Ser Ala Ala′ repeating units”
  SITE (648)..(682): /note=“This region may encompass 1-5 ′Gly Gly Gly Gly Ser Ala Ala′ repeating units”
  SITE (683)..(689): /note=“This region may or may not be present”
  SITE (690)..(699): /note=“This region may encompass 0-2 ′Gly Gly Gly Gly Ser′ repeating units”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAAPYAYWMRGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELK

SEQ ID NO: 34 - protein, 933 aa, Artificial Sequence
  source: /note=“Description of Artificial Sequence Synthetic polypeptide”
  SITE (453)..(487): /note=“This region may encompass 0-5 ′Gly Gly Gly Gly Ser Ala Ala′ repeating units”
  SITE (728)..(762): /note=“This region may encompass 1-5 ′Gly Gly Gly Gly Ser Ala Ala′ repeating units”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAAQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSAAGGGGSAAGGGGSAAGGGGSAAGGGGSAAPYAYWMRGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 35 - protein, 214 aa, Artificial Sequence
  source: /note=“Description of Artificial Sequence Synthetic polypeptide”
DIQMTQSPSSLSASVGDRVTITCGTSEDIINYLNWYQQKPGKAPKLLIYHTSRLQSGVPSRFSGSGSGTDFTLTISSLQPEDFATYYCQQGYTLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 818 aa, Artificial Sequence
  source: /note=“Description of Artificial Sequence Synthetic polypeptide”
  SITE (449)..(463): /note=“This region may encompass 0-3 ′Gly Gly Gly Gly Ser′ repeating units”
  SITE (624)..(658): /note=“This region may encompass 1-5 ′Gly Gly Gly Gly Ser Ala Ala′ repeating units”
  SITE (624)..(818): /note=“This region may or may not be present”
EVQLVQSGAEVKKPGASVKVSCKASGYTFTSYVIHWVRQRPGQGLAWMGYINPYNDGTKYNERFKGKVTITSDRSTSTVYMELSSLRSEDTAVYLCGREGIRYYGLLGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 37 - protein, 938 aa, Artificial Sequence
  source: /note=“Description of Artificial Sequence Synthetic polypeptide”
  SITE (380)..(407): /note=“This region may encompass 1-4 ′Gly Gly Gly Gly Ser Ala Ala′ repeating units”
  SITE (648)..(682): /note=“This region may encompass 1-5 ′Gly Gly Gly Gly Ser Ala Ala′ repeating units”
  SITE (683)..(688): /note=“This region may or may not be present”
  SITE (689)..(698): /note=“This region may encompass 0-2 ′Gly Gly Gly Gly Ser′ repeating units”
  SITE (648)..(938): /note=“This region may or may not be present”
EVQLVQSGAEVKKPGASVKVSCKASGYTFTSYVIHWVRQRPGQGLAWMGYINPYNDGTKYNERFKGKVTITSDRSTSTVYMELSSLRSEDTAVYLCGREGIRYYGLLGDYWGQGTLVTVSSTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSAAGGGGSAAGGGGSAAGGGGSAADIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSAAGGGGSAAGGGGSAAGGGGSAAGGGGSAAGPLGVRGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 38 - protein, 1007 aa, Artificial Sequence
  source: /note=“Description of Artificial Sequence Synthetic polypeptide”
  SITE (449)..(476): /note=“This region may encompass 1-4 ′Gly Gly Gly Gly Ser Ala Ala′ repeating units”
  SITE (717)..(751): /note=“This region may encompass 1-5 ′Gly Gly Gly Gly Ser Ala Ala′ repeating units”
  SITE (752)..(757): /note=“This region may or may not be present”
  SITE (758)..(767): /note=“This region may encompass 0-2 ′Gly Gly Gly Gly Ser′ repeating units”
  SITE (717)..(1007): /note=“This region may or may not be present”
EVQLVQSGAEVKKPGASVKVSCKASGYTFTSYVIHWVRQRPGQGLAWMGYINPYNDGTKYNERFKGKVTITSDRSTSTVYMELSSLRSEDTAVYLCGREGIRYYGLLGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSAAGGGGSAAGGGGSAAGGGGSAAQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSAAGGGGSAAGGGGSAAGGGGSAAGGGGSAAGPLGVRGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELK

SEQ ID NO: 39 - protein, 935 aa, Artificial Sequence
  source: /note=“Description of Artificial Sequence Synthetic polypeptide”
  SITE (449)..(483): /note=“This region may encompass 0-5 ′Gly Gly Gly Gly Ser Ala Ala′ repeating units”
  SITE (644)..(678): /note=“This region may encompass 0-5 ′Gly Gly Gly Gly Ser Ala Ala′ repeating units”
  SITE (679)..(685): /note=“This region may or may not be present”
  SITE (686)..(695): /note=“This region may encompass 0-2 ′Gly Gly Gly Gly Ser′ repeating units”
EVQLVQSGAEVKKPGASVKVSCKASGYTFTSYVIHWVRQRPGQGLAWMGYINPYNDGTKYNERFKGKVTITSDRSTSTVYMELSSLRSEDTAVYLCGREGIRYYGLLGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAAPYAYWMRGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 40 - protein, 908 aa, Artificial Sequence
  source: /note=“Description of Artificial Sequence Synthetic polypeptide”
  SITE (449)..(473): /note=“This region may encompass 0-5 ′Gly Gly Gly Gly Ser′ repeating units”
  SITE (714)..(748): /note=“This region may encompass 0-5 ′Gly Gly Gly Gly Ser Ala Ala′ repeating units”
EVQLVQSGAEVKKPGASVKVSCKASGYTFTSYVIHWVRQRPGQGLAWMGYINPYNDGTKYNERFKGKVTITSDRSTSTVYMELSSLRSEDTAVYLCGREGIRYYGLLGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 41 - protein, 935 aa, Artificial Sequence
  source: /note=“Description of Artificial Sequence Synthetic polypeptide”
  SITE (449)..(483): /note=“This region may encompass 0-5 ′Gly Gly Gly Gly Ser Ala Ala′ repeating units”
  SITE (644)..(678): /note=“This region may encompass 1-5 ′Gly Gly Gly Gly Ser Ala Ala′ repeating units”
  SITE (679)..(685): /note=“This region may or may not be present”
  SITE (686)..(695): /note=“This region may encompass 0-2 ′Gly Gly Gly Gly Ser′ repeating units”
EVQLVQSGAEVKKPGASVKVSCKASGYTFTSYVIHWVRQRPGQGLAWMGYINPYNDGTKYNERFKGKVTITSDRSTSTVYMELSSLRSEDTAVYLCGREGIRYYGLLGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAAPYAYWMRGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELK

SEQ ID NO: 42 - protein, 918 aa, Artificial Sequence
  source: /note=“Description of Artificial Sequence Synthetic polypeptide”
  SITE (449)..(483): /note=“This region may encompass 0-5 ′Gly Gly Gly Gly Ser Ala Ala′ repeating units”
  SITE (724)..(758): /note=“This region may encompass 1-5 ′Gly Gly Gly Gly Ser Ala Ala′ repeating units”
EVQLVQSGAEVKKPGASVKVSCKASGYTFTSYVIHWVRQRPGQGLAWMGYINPYNDGTKYNERFKGKVTITSDRSTSTVYMELSSLRSEDTAVYLCGREGIRYYGLLGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAAQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 43 - protein, 214 aa, Artificial Sequence
  source: /note=“Description of Artificial Sequence Synthetic polypeptide”
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 813 aa, Artificial Sequence
  source: /note=“Description of Artificial Sequence Synthetic polypeptide”
  SITE (444)..(458): /note=“This region may encompass 0-3 ′Gly Gly Gly Gly Ser′ repeating units”
  SITE (619)..(653): /note=“This region may encompass 1-5 ′Gly Gly Gly Gly Ser Ala Ala′ repeating units”
  SITE (619)..(813): /note=“This region may or may not be present”
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 45 - protein, 1003 aa, Artificial Sequence
  source: /note=“Description of Artificial Sequence Synthetic polypeptide”
  SITE (444)..(471): /note=“This region may encompass 1-4 ′Gly Gly Gly Gly Ser Ala Ala′ repeating units”
  SITE (712)..(746): /note=“This region may encompass 1-5 ′Gly Gly Gly Gly Ser Ala Ala′ repeating units”
  SITE (747)..(753): /note=“This region may or may not be present”
  SITE (754)..(763): /note=“This region may encompass 0-2 ′Gly Gly Gly Gly Ser′ repeating units”
  SITE (712)..(1003): /note=“This region may or may not be present”
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSAAGGGGSAAGGGGSAAGGGGSAADIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSAAGGGGSAAGGGGSAAGGGGSAAGGGGSAAPYAYWMRGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 46 - protein, 1003 aa, Artificial Sequence
  source: /note=“Description of Artificial Sequence Synthetic polypeptide”
  SITE (444)..(471): /note=“This region may encompass 1-4 ′Gly Gly Gly Gly Ser Ala Ala′ repeating units”
  SITE (712)..(746): /note=“This region may encompass 1-5 ′Gly Gly Gly Gly Ser Ala Ala′ repeating units”
  SITE (747)..(753): /note=“This region may or may not be present”
  SITE (754)..(763): /note=“This region may encompass 0-2 ′Gly Gly Gly Gly Ser′ repeating units”
  SITE (712)..(1003): /note=“This region may or may not be present”
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSAAGGGGSAAGGGGSAAGGGGSAAQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSAAGGGGSAAGGGGSAAGGGGSAAGGGGSAAPYAYWMRGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELK

SEQ ID NO: 47 - protein, 925 aa, Artificial Sequence
  source: /note=“Description of Artificial Sequence Synthetic polypeptide”
  SITE (444)..(478): /note=“This region may encompass 0-5 ′Gly Gly Gly Gly Ser Ala Ala′ repeating units”
  SITE (639)..(673): /note=“This region may encompass 0-5 ′Gly Gly Gly Gly Ser Ala Ala′ repeating units”
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAAPYAYWMRGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 48 - protein, 903 aa, Artificial Sequence
  source: /note=“Description of Artificial Sequence Synthetic polypeptide”
  SITE (444)..(468): /note=“This region may encompass 0-5 ′Gly Gly Gly Gly Ser′ repeating units”
  SITE (709)..(743): /note=“This region may encompass 0-5 ′Gly Gly Gly Gly Ser Ala Ala′ repeating units”
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 49 - protein, 924 aa, Artificial Sequence
  source: /note=“Description of Artificial Sequence Synthetic polypeptide”
  SITE (444)..(478): /note=“This region may encompass 0-5 ′Gly Gly Gly Gly Ser Ala Ala′ repeating units”
  SITE (719)..(753): /note=“This region may encompass 1-5 ′Gly Gly Gly Gly Ser Ala Ala′ repeating units”
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAAQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSAAGGGGSAAGGGGSAAGGGGSAAGGGGSAAPYAYWMRGGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 50 - protein, 924 aa, Artificial Sequence; identical to SEQ ID NO: 49
  source: /note=“Description of Artificial Sequence Synthetic polypeptide”
  SITE (444)..(478): /note=“This region may encompass 0-5 ′Gly Gly Gly Gly Ser Ala Ala′ repeating units”
  SITE (719)..(753): /note=“This region may encompass 1-5 ′Gly Gly Gly Gly Ser Ala Ala′ repeating units”

SEQ ID NO: 51 - protein, 117 aa, Artificial Sequence [sequence omitted]

SEQ ID NO: 52 - protein, 108 aa, Artificial Sequence [sequence omitted]

SEQ ID NO: 53 - protein, 215 aa, Artificial Sequence
  source: /note=“Description of Artificial Sequence Synthetic polypeptide”
EIVLTQSPGTLSLSPGERATLSCRASQSVSSSFFAWYQQKPGQAPRLLIYGASSRATGIPDRLSGSGSGTDFTLTITRLEPEDFAVYYCQQYDSSAITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 814 aa, Artificial Sequence
  source: /note=“Description of Artificial Sequence Synthetic polypeptide”
  SITE (445)..(459): /note=“This region may encompass 0-3 ′Gly Gly Gly Gly Ser′ repeating units”
  SITE (620)..(654): /note=“This region may encompass 1-5 ′Gly Gly Gly Gly Ser Ala Ala′ repeating units”
  SITE (620)..(814): /note=“This region may or may not be present”
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 55 - protein, 1004 aa, Artificial Sequence
  source: /note=“Description of Artificial Sequence Synthetic polypeptide”
  SITE (445)..(472): /note=“This region may encompass 1-4 ′Gly Gly Gly Gly Ser Ala Ala′ repeating units”
  SITE (713)..(747): /note=“This region may encompass 1-5 ′Gly Gly Gly Gly Ser Ala Ala′ repeating units”
  SITE (748)..(754): /note=“This region may or may not be present”
  SITE (755)..(764): /note=“This region may encompass 0-2 ′Gly Gly Gly Gly Ser′ repeating units”
  SITE (713)..(1004): /note=“This region may or may not be present”
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSAAGGGGSAAGGGGSAAGGGGSAADIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSAAGGGGSAAGGGGSAAGGGGSAAGGGGSAAPYAYWMRGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 56 - protein, 1004 aa, Artificial Sequence
  source: /note=“Description of Artificial Sequence Synthetic polypeptide”
  SITE (445)..(472): /note=“This region may encompass 1-4 ′Gly Gly Gly Gly Ser Ala Ala′ repeating units”
  SITE (713)..(747): /note=“This region may encompass 1-5 ′Gly Gly Gly Gly Ser Ala Ala′ repeating units”
  SITE (748)..(754): /note=“This region may or may not be present”
  SITE (755)..(764): /note=“This region may encompass 0-2 ′Gly Gly Gly Gly Ser′ repeating units”
  SITE (713)..(1004): /note=“This region may or may not be present”
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSAAGGGGSAAGGGGSAAGGGGSAAQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSAAGGGGSAAGGGGSAAGGGGSAAGGGGSAAPYAYWMRGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELK

SEQ ID NO: 57 - protein, 926 aa, Artificial Sequence
  source: /note=“Description of Artificial Sequence Synthetic polypeptide”
  SITE (445)..(479): /note=“This region may encompass 0-5 ′Gly Gly Gly Gly Ser Ala Ala′ repeating units”
  SITE (640)..(674): /note=“This region may encompass 0-5 ′Gly Gly Gly Gly Ser Ala Ala′ repeating units”
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAAPYAYWMRGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 58 - protein, 904 aa, Artificial Sequence
  source: /note=“Description of Artificial Sequence Synthetic polypeptide”
  SITE (445)..(469): /note=“This region may encompass 0-5 ′Gly Gly Gly Gly Ser′ repeating units”
  SITE (710)..(744): /note=“This region may encompass 0-5 ′Gly Gly Gly Gly Ser Ala Ala′ repeating units”
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 59 - protein, 926 aa, Artificial Sequence
  source: /note=“Description of Artificial Sequence Synthetic polypeptide”
  SITE (445)..(479): /note=“This region may encompass 0-5 ′Gly Gly Gly Gly Ser Ala Ala′ repeating units”
  SITE (640)..(674): /note=“This region may encompass 1-5 ′Gly Gly Gly Gly Ser Ala Ala′ repeating units”
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAAPYAYWMRGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELK

SEQ ID NO: 60 - protein, 914 aa, Artificial Sequence
  source: /note=“Description of Artificial Sequence Synthetic polypeptide”
  SITE (445)..(479): /note=“This region may encompass 0-5 ′Gly Gly Gly Gly Ser Ala Ala′ repeating units”
  SITE (720)..(754): /note=“This region may encompass 1-5 ′Gly Gly Gly Gly Ser Ala Ala′ repeating units”
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAAQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 61 - protein, 124 aa, Artificial Sequence
  source: /note=“Description of Artificial Sequence Synthetic polypeptide”
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSD

SEQ ID NO: 62 - protein, 7 aa, Artificial Sequence
  source: /note=“Description of Artificial Sequence Synthetic peptide”
RGFFRGG

SEQ ID NO: 63 - protein, 730 aa, Artificial Sequence
  source: /note=“Description of Artificial Sequence Synthetic polypeptide”
  SITE (361)..(375): /note=“This region may encompass 0-3 ′Gly Gly Gly Gly Ser′ repeating units”
  SITE (536)..(570): /note=“This region may encompass 1-5 ′Gly Gly Gly Gly Ser Ala Ala′ repeating units”
  SITE (536)..(730): /note=“This region may or may not be present”
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSAESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 64 - protein, 920 aa, Artificial Sequence
  source: /note=“Description of Artificial Sequence Synthetic polypeptide”
  SITE (361)..(388): /note=“This region may encompass 1-4 ′Gly Gly Gly Gly Ser Ala Ala′ repeating units”
  SITE (629)..(663): /note=“This region may encompass 1-5 ′Gly Gly Gly Gly Ser Ala Ala′ repeating units”
  SITE (664)..(670): /note=“This region may or may not be present”
  SITE (671)..(680): /note=“This region may encompass 0-2 ′Gly Gly Gly Gly Ser′ repeating units”
  SITE (629)..(920): /note=“This region may or may not be present”
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSAESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSAAGGGGSAAGGGGSAAGGGGSAADIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSAAGGGGSAAGGGGSAAGGGGSAAGGGGSAAPYAYWMRGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 65 - protein, 920 aa, Artificial Sequence
  source: /note=“Description of Artificial Sequence Synthetic polypeptide”
  SITE (361)..(388): /note=“This region may encompass 1-4 ′Gly Gly Gly Gly Ser Ala Ala′ repeating units”
  SITE (629)..(663): /note=“This region may encompass 1-5 ′Gly Gly Gly Gly Ser Ala Ala′ repeating units”
  SITE (664)..(670): /note=“This region may or may not be present”
  SITE (671)..(680): /note=“This region may encompass 0-2 ′Gly Gly Gly Gly Ser′ repeating units”
  SITE (629)..(920): /note=“This region may or may not be present”
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSAESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSAAGGGGSAAGGGGSAAGGGGSAAQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSAAGGGGSAAGGGGSAAGGGGSAAGGGGSAAPYAYWMRGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELK

SEQ ID NO: 66 - protein, 842 aa, Artificial Sequence
  source: /note=“Description of Artificial Sequence Synthetic polypeptide”
  SITE (361)..(395): /note=“This region may encompass 0-5 ′Gly Gly Gly Gly Ser Ala Ala′ repeating units”
  SITE (556)..(590): /note=“This region may encompass 0-5 ′Gly Gly Gly Gly Ser Ala Ala′ repeating units”
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSAESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAAPYAYWMRGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 67 - protein, 820 aa, Artificial Sequence
  source: /note=“Description of Artificial Sequence Synthetic polypeptide”
  SITE (361)..(385): /note=“This region may encompass 0-5 ′Gly Gly Gly Gly Ser′ repeating units”
  SITE (626)..(660): /note=“This region may encompass 0-5 ′Gly Gly Gly Gly Ser Ala Ala′ repeating units”
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSAESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 68 - protein, 842 aa, Artificial Sequence
  source: /note=“Description of Artificial Sequence Synthetic polypeptide”
  SITE (361)..(395): /note=“This region may encompass 0-5 ′Gly Gly Gly Gly Ser Ala Ala′ repeating units”
  SITE (556)..(590): /note=“This region may encompass 1-5 ′Gly Gly Gly Gly Ser Ala Ala′ repeating units”
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSAESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAAPYAYWMRGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELK

SEQ ID NO: 69 - protein, 830 aa, Artificial Sequence
  source: /note=“Description of Artificial Sequence Synthetic polypeptide”
  SITE (361)..(395): /note=“This region may encompass 0-5 ′Gly Gly Gly Gly Ser Ala Ala′ repeating units”
  SITE (636)..(670): /note=“This region may encompass 1-5 ′Gly Gly Gly Gly Ser Ala Ala′ repeating units”
MHVAQPAVVLASSRGIASFVCEYASPGKYTEVRVTVLRQADSQVTEVCAATYMMGNELTFLDDSICTGTSSGNQVNLTIQGLRAMDTGLYICKVELMYPPPYYEGIGNGTQIYVIDPEPCPDSDQEPKSSAESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAAQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 70 - protein, 841 aa, Artificial Sequence
  source: /note=“Description of Artificial Sequence Synthetic polypeptide”
  SITE (472)..(486): /note=“This region may encompass 0-3 ′Gly Gly Gly Gly Ser′ repeating units”
  SITE (647)..(681): /note=“This region may encompass 0-5 ′Gly Gly Gly Gly Ser Ala Ala′ repeating units”
  SITE (647)..(841): /note=“This region may or may not be present”
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDQEPKSSAESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 71 - protein, 1030 aa, Artificial Sequence
  source: /note=“Description of Artificial Sequence Synthetic polypeptide”
  SITE (472)..(499): /note=“This region may encompass 1-4 ′Gly Gly Gly Gly Ser Ala Ala′ repeating units”
  SITE (740)..(774): /note=“This region may encompass 1-5 ′Gly Gly Gly Gly Ser Ala Ala′ repeating units”
  SITE (775)..(780): /note=“This region may or may not be present”
  SITE (781)..(790): /note=“This region may encompass 0-2 ′Gly Gly Gly Gly Ser′ repeating units”
  SITE (740)..(1030): /note=“This region may or may not be present”
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDQEPKSSAESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSAAGGGGSAAGGGGSAAGGGGSAADIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSAAGGGGSAAGGGGSAAGGGGSAAGGGGSAAGPLGVRGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 72 - protein, 1031 aa, Artificial Sequence
  source: /note=“Description of Artificial Sequence Synthetic polypeptide”
  SITE (472)..(499): /note=“This region may encompass 1-4 ′Gly Gly Gly Gly Ser Ala Ala′ repeating units”
  SITE (740)..(774): /note=“This region may encompass 1-5 ′Gly Gly Gly Gly Ser Ala Ala′ repeating units”
  SITE (775)..(781): /note=“This region may or may not be present”
  SITE (782)..(791): /note=“This region may encompass 0-2 ′Gly Gly Gly Gly Ser′ repeating units”
  SITE (740)..(1031): /note=“This region may or may not be present”
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDQEPKSSAESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSAAGGGGSAAGGGGSAAGGGGSAAQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSAAGGGGSAAGGGGSAAGGGGSAAGGGGSAAPYAYWMRGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELK

SEQ ID NO: 73 - protein, 1001 aa, Artificial Sequence
  source: /note=“Description of Artificial Sequence Synthetic polypeptide”
  SITE (472)..(506): /note=“This region may encompass 0-5 ′Gly Gly Gly Gly Ser Ala Ala′ repeating units”
  SITE (667)..(701): /note=“This region may encompass 0-5 ′Gly Gly Gly Gly Ser Ala Ala′ repeating units”
  SITE (702)..(736): /note=“This region may encompass 0-5 ′Pro Tyr Ala Tyr Trp Met Arg′ repeating units”
  SITE (737)..(761): /note=“This region may encompass 0-5 ′Gly Gly Gly Gly Ser′ repeating units”
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDQEPKSSAESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAAPYAYWMRPYAYWMRPYAYWMRPYAYWMRPYAYWMRGGGGSGGGGSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 74 - protein, 1001 aa, Artificial Sequence
  source: /note=“Description of Artificial Sequence Synthetic polypeptide”
  SITE (472)..(506): /note=“This region may encompass 0-5 ′Gly Gly Gly Gly Ser Ala Ala′ repeating units”
  SITE (667)..(701): /note=“This region may encompass 1-5 ′Gly Gly Gly Gly Ser Ala Ala′ repeating units”
  SITE (702)..(736): /note=“This region may encompass 0-5 ′Pro Tyr Ala Tyr Trp Met Arg′ repeating units”
  SITE (737)..(761): /note=“This region may encompass 0-5 ′Gly Gly Gly Gly Ser′ repeating units”
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDQEPKSSAESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSAAGGGGSAAGGGGSAAGGGGSAAGGGGSAASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSAAGGGGSAAGGGGSAAGGGGSAAGGGGSAAPYAYWMRPYAYWMRPYAYWMRPYAYWMRPYAYWMRGGGGSGGGGSGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELK

SEQ ID NO: 75 - protein, 13 aa, Artificial Sequence
  source: /note=“Description of Artificial Sequence Synthetic peptide”
GGSLSGRSDNHGS

SEQ ID NO: 76 - protein, 7 aa, Artificial Sequence
  source: /note=“Description of Artificial Sequence Synthetic peptide”
PYAYWMR

SEQ ID NO: 77 - protein, 6 aa, Artificial Sequence
  source: /note=“Description of Artificial Sequence Synthetic peptide”
GPLGVR

SEQ ID NO: 78 - protein, 7 aa, Artificial Sequence
  source: /note=“Description of Artificial Sequence Synthetic peptide”
PLGMWSR

SEQ ID NO: 79 - protein, 7 aa, Artificial Sequence
  source: /note=“Description of Artificial Sequence Synthetic peptide”
PLGLWAR

SEQ ID NO: 80 - protein, 8 aa, Artificial Sequence
  source: /note=“Description of Artificial Sequence Synthetic peptide”
PQGIAGQR

SEQ ID NO: 81 - protein, 6 aa, Artificial Sequence
  source: /note=“Description of Artificial Sequence Synthetic peptide”
PLGLAG

SEQ ID NO: 82 - protein, 6 aa, Artificial Sequence
  source: /note=“Description of Artificial Sequence Synthetic peptide”
LALGPR

SEQ ID NO: 83 - protein, 10 aa, Artificial Sequence
  source: /note=“Description of Artificial Sequence Synthetic peptide”
GGPLGMLSQS

SEQ ID NO: 84 - protein, 9 aa, Artificial Sequence
  source: /note=“Description of Artificial Sequence Synthetic peptide”
GGGGRRGGS

SEQ ID NO: 85 - protein, 8 aa, Artificial Sequence
  source: /note=“Description of Artificial Sequence Synthetic peptide”
TGRGPSWV

SEQ ID NO: 86 - protein, 8 aa, Artificial Sequence
  source: /note=“Description of Artificial Sequence Synthetic peptide”
SARGPSRW

SEQ ID NO: 87 - protein, 8 aa, Artificial Sequence
  source: /note=“Description of Artificial Sequence Synthetic peptide”
TARGPSFK

SEQ ID NO: 88 - protein, 7 aa, Artificial Sequence
  source: /note=“Description of Artificial Sequence Synthetic peptide”
TARGPSW

SEQ ID NO: 89 - protein, 8 aa, Artificial Sequence
  source: /note=“Description of Artificial Sequence Synthetic peptide”
GGWHTGRN

SEQ ID NO: 90 - protein, 8 aa, Artificial Sequence
  source: /note=“Description of Artificial Sequence Synthetic peptide”
HTGRSGAL

SEQ ID NO: 91 - protein, 8 aa, Artificial Sequence
  source: /note=“Description of Artificial Sequence Synthetic peptide”
PLTGRSGG

SEQ ID NO: 92 - protein, 7 aa, Artificial Sequence
  source: /note=“Description of Artificial Sequence Synthetic peptide”
LTGRSGA

SEQ ID NO: 93 - protein, 8 aa, Artificial Sequence
  source: /note=“Description of Artificial Sequence Synthetic peptide”
RQARVVNG

SEQ ID NO: 94 - protein, 18 aa, Artificial Sequence
  source: /note=“Description of Artificial Sequence Synthetic peptide”
VHMPLGFLGPRQARVVNA

SEQ ID NO: 95 - protein, 6 aa, Artificial Sequence
  source: /note=“Description of Artificial Sequence Synthetic peptide”
QEPKSS

SEQ ID NO: 96 - protein, 15 aa, Artificial Sequence
  source: /note=“Description of Artificial Sequence Synthetic peptide”
  SITE (1)..(15): /note=“This sequence may encompass 1-3 ′Gly Gly Gly Gly Ser′ repeating units”
GGGGSGGGGSGGGGS

SEQ ID NO: 97 - protein, 12 aa, Artificial Sequence
  source: /note=“Description of Artificial Sequence Synthetic peptide”
GSAGSAAGSGEF

SEQ ID NO: 98 - protein, 42 aa, Artificial Sequence
  source: /note=“Description of Artificial Sequence Synthetic polypeptide”
  SITE (1)..(15): /note=“This region may encompass 1-3 ′Gly Gly Gly Gly Ser′ repeating units”
  SITE (28)..(42): /note=“This region may encompass 1-3 ′Gly Gly Gly Gly Ser′ repeating units”
GGGGSGGGGSGGGGSGSAGSAAGSGEFGGGGSGGGGSGGGGS

SEQ ID NO: 99 - protein, 32 aa, Artificial Sequence
  source: /note=“Description of Artificial Sequence Synthetic polypeptide”
  SITE (1)..(15): /note=“This region may encompass 2-3 ′Gly Gly Gly Gly Ser′ repeating units”
  SITE (18)..(32): /note=“This region may encompass 2-3 ′Gly Gly Gly Gly Ser′ repeating units”
GGGGSGGGGSGGGGSAAGGGGSGGGGSGGGGS

SEQ ID NO: 100 - protein, 619 aa, Artificial Sequence
  source: /note=“Description of Artificial Sequence Synthetic polypeptide”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAPYAYWMRSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 101 - protein, 383 aa, Artificial Sequence
  source: /note=“Description of Artificial Sequence Synthetic polypeptide”
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 558 aa, Artificial Sequence
  source: /note=“Description of Artificial Sequence Synthetic polypeptide”
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 103 - protein, 821 aa, Artificial Sequence
  source: /note=“Description of Artificial Sequence Synthetic polypeptide”
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSGGGGSAAGGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 104 - protein, 821 aa, Artificial Sequence
  source: /note=“Description of Artificial Sequence Synthetic polypeptide”
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSGGGGSAAGGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELK

SEQ ID NO: 105 - protein, 821 aa, Artificial Sequence
  source: /note=“Description of Artificial Sequence Synthetic polypeptide”
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSAAGGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 106 - protein, 821 aa, Artificial Sequence
  source: /note=“Description of Artificial Sequence Synthetic polypeptide”
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSAAGGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 107 - protein, 558 aa, Artificial Sequence
  source: /note=“Description of Artificial Sequence Synthetic polypeptide”
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGAGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 108 - protein, 240 aa, Artificial Sequence
  source: /note=“Description of Artificial Sequence Synthetic polypeptide”
MDMRVPAQLLGLLLLWLPGAKCDIVLTQSPDSLAVSLGERATINCRASESVDTFDYSFLHWYQQKPGQPPKLLIYRASNLESGVPDRFSGSGSGTDFTLTISSLQAEDVAVYYCQQSNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 465 aa, Artificial Sequence
  source: /note=“Description of Artificial Sequence Synthetic polypeptide”
MGVKVLFALICIAVAEAEVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 906 aa, Artificial Sequence
  source: /note=“Description of Artificial Sequence Synthetic polypeptide”
MGVKVLFALICIAVAEAEVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSGGGGSGPLGVRGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 111 - protein, 903 aa, Artificial Sequence
  source: /note=“Description of Artificial Sequence Synthetic polypeptide”
MGVKVLFALICIAVAEAEVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSAAGGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 112 - protein, 640 aa, Artificial Sequence
  source: /note=“Description of Artificial Sequence Synthetic polypeptide”
MGVKVLFALICIAVAEAEVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGAGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRN

SEQ ID NO: 113 - protein, 907 aa, Artificial Sequence
  source: /note=“Description of Artificial Sequence Synthetic polypeptide”
MGVKVLFALICIAVAEAEVQLVESGGGLVQPGGSLKLSCAASGFTFSTYAMHWVRQASGKGLEWVGRIRTKSSNYATYYAASVKGRFTISRDDSKNTAYLQMNSLKTEDTAVYYCTRDMGIRRQFAYWGQGTLVTVSSASTKGPSVFPLAPCSRSTSESTAALACLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNGGGGSGGGGSPYAYWMRGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSS

SEQ ID NO: 114 - protein, 912 aa, Artificial Sequence
  source: /note=“Description of Artificial Sequence Synthetic polypeptide”
MGVKVLFALICIAVAEAQVQLVQSGAEVKKPGASVKVSCKASGYTFTSYGINWVRQAPGQGLEWMGWISVYSGNTNYAQKVQGRVTMTADTSTSTAYMDLRSLRSDDTAVYYCAREGSSSSGDYYYGMDVWGQGTTVTVSSASTKGPSVFPLAPSSKSTSGGTAALACLVKDYFPEPVTVSWNSGALTSGVHTFPAVLQSSGLYSLSSVVTVPSSSLGTQTYICNVNHKPSNTKVDKKAEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNAGGGGSGGGGSPYAYWMRGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELK

SEQ ID NO: 115 - protein, 888 aa, Artificial Sequence
  source: /note=“Description of Artificial Sequence Synthetic polypeptide”
QVQLVQSGAEVKKPGASVKVSCKASGYTFTSYGINWVRQAPGQGLEWMGWISVYSGNTNYAQKVQGRVTMTADTSTSTAYMDLRSLRSDDTAVYYCAREGSSSSGDYYYGMDVWGQGTTVTVSSASTKGPSVFPLAPSSKSTSGGTAALACLVKDYFPEPVTVSWNSGALTSGVHTFPAVLQSSGLYSLSSVVTVPSSSLGTQTYICNVNHKPSNTKVDKKAEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSQVQLVESGGGVVQPGRSLRLSCAASGFTFSDYHMAWVRQAPGKGLEWVASITLDATYTYYRDSVRGRFTISRDNSKNTLYLQMNSLRAEDTAVYYCARHRGFSVWLDYWGQGTLVTVSSGGGGSGGGGSGGGGSDIQMTQSPSSLSASVGDRVTITCKTSQNIFENLAWYQQKPGKAPKLLIYNASPLQAGVPSRFSGSGSGTDFTLTISSLQPEDFATYYCHQYYSGYTFGPGTKLELKAGGGGSPYAYWMRGGGGSGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRSLPCENKSKAVEQVKNAFNKLQEKGIYKAMSEFDIFINYIEAYMTMKIRNA

SEQ ID NO: 116 - protein, 219 aa, Artificial Sequence
  source: /note=“Description of Artificial Sequence Synthetic polypeptide”
DIVMTQTPLSLSVTPGQPASISCKSSQSLLHTDGTTYLYWYLQKPGQPPQLLIYEVSNRFSGVPDRFSGSGSGTDFTLKISRVEAEDVGIYYCMQNIQLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 5 aa, Artificial Sequence
  source: /note=“Description of Artificial Sequence Synthetic peptide”
GGGGS

SEQ ID NO: 118 - protein, 10 aa, Artificial Sequence
  source: /note=“Description of Artificial Sequence Synthetic peptide”
GGGGSGGGGS

SEQ ID NO: 119 - protein, 15 aa, Artificial Sequence
  source: /note=“Description of Artificial Sequence Synthetic peptide”
GGGGSGGGGSGGGGS

SEQ ID NO: 120 - protein, 22 aa, Artificial Sequence
  source: /note=“Description of Artificial Sequence Synthetic peptide”
GGGGSGGGGSAAGGGGSGGGGS

(19) United States
(12) Patent Application Publication
(10) Pub. No.: US 2024/0076331 A1
(43) Pub. Date: Mar. 7, 2024

(54) Chimeric Molecules Comprising an IL-10 or TGF-Beta Agonist Polypeptide

(71) Applicant: ASKGENE PHARMA, INC., Camarillo, CA (US)

(72) Inventor: Yuefeng LU, Moorpark, CA (US)

(73) Assignee: ASKGENE PHARMA, INC., Camarillo, CA (US)

(21) Appl. No.: 18/259,706

(22) PCT Filed: Feb. 1, 2022
(86) PCT No.: PCT/US2022/014801

§ 371 Date: Jun. 28, 2023

Related U.S. Application Data
(60) Provisional application No. 63/143,954, filed on Feb. 1, 2021.

(51) Int. Cl. C07K 14/495; C07K 14/54

(52) U.S. Cl. CPC C07K 14/495; C07K 14/5428; A61K 38/00; C07K 2319/50

(57) ABSTRACT

The present disclosure relates to prodrugs or chimeric molecules which comprise a carrier moiety, a cytokine moiety selected from an IL-10 agonist polypeptide and a TGF-β agonist polypeptide, and a masking moiety that binds to said cytokine moiety and inhibits its biological activity. Further included in the present disclosure are methods of making and using the novel prodrugs or chimeric molecules.

### CROSS-REFERENCE TO RELATED APPLICATION

The present application claims priority from U.S. Provisional Application No. 63/143,954, filed on Feb. 1, 2021, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jan. 28, 2022, is named 025471_WO016_SL.txt and is 501,203 bytes in size.

### BACKGROUND OF THE INVENTION

IL-10 is an anti-inflammatory cytokine. It inhibits cytokine (e.g., IFN-γ) production by Th1 cells via its action on antigen-presenting cell (APC) functions, including inhibition of cytokine production by activated macrophages and dendritic cells (Bogdan et al., J Exp Med. (1991) 174(6):1549-55; Fiorentino et. al., J Immunol. (1991) 146(10):3444-51; and Macatonia et al., J Immunol. (1993) 150(9):3755-65). IL-10 is also known to prevent antigen-specific proliferation of human T cells through inhibition of antigen-presenting capacity of monocytes via downregulation of class II MHC expression (de Waal Malefyt R et al., J Exp Med. (1991) 174(4):915-24).

Transforming growth factor β (TGF-β) is a key pleiotropic cytokine involved in controlling immune cell functions relating to both suppressive and inflammatory immune responses. TGF-β inhibits cytotoxic T lymphocyte (CTL), Th1-, and Th2-cell differentiation while promoting peripheral (p)Treg-, Th17-, Th9-, and Tfh-cell generation, and T-cell tissue residence in response to immune challenges (Sanjabi et al., Cold Spring Harb Perspect Biol. (2017) 9(6):a0222336).

Given that IL-10 and TGF-β have immune-suppressive activities, these cytokines may potentially form the basis of therapies for autoimmune and inflammation diseases (Wang et al., Cold Spring Harb Perspect Biol. (2019) 11(2):a028548; Worthington et al., Immunobiology (2012) 217(12):1259-65). However, these cytokines have been found to have short half-lives in vivo, have high toxicity, and lack specificity for immune cells or disease site selectivity. Thus, there is a need to develop novel IL-10 and TGF-β cytokine therapeutics that have longer half-lives, cause fewer adverse effects, and provide better target specificity.

### SUMMARY OF THE INVENTION

The present disclosure provides a prodrug comprising a cytokine moiety, a masking moiety, and a carrier moiety, wherein the masking moiety binds to the cytokine moiety and inhibits a biological activity of the cytokine moiety, the cytokine moiety comprises an interleukin-10 (IL-10) agonist polypeptide or a transforming factor beta (TGF-β) agonist polypeptide and is fused to the carrier moiety or the masking moiety, the carrier moiety binds to an antigen expressed on the surface of an immune cell; wherein the immune cell expresses a receptor for the cytokine moiety; and the masking moiety is fused to the cytokine moiety or to the carrier moiety, optionally through a peptide linker.

In particular embodiments, the IL-10 agonist polypeptide comprises SEQ ID NO: 1, 2, or 3, or an amino acid sequence that is at least 90% identical to SEQ ID NO: 1, 2, or 3.

In some embodiments, the TGF-β is selected from a TGF-β1 agonist polypeptide, a TGF-β2 agonist polypeptide, and a TGF-β3 agonist polypeptide. In some embodiments, the cytokine moiety comprises a human TGF-β agonist polypeptide comprising SEQ ID NO: 7, or an amino acid sequence that is at least 90% identical to SEQ ID NO: 7.

In some embodiments, the cytokine moiety is fused to the carrier moiety directly or via a non-cleavable or cleavable peptide linker and the masking moiety is fused to the carrier moiety directly or via a non-cleavable or cleavable peptide linker. In other embodiments, the prodrug further comprises a second cytokine moiety fused to the C-terminus of the cytokine moiety. In additional embodiments, the prodrug further comprises a second masking moiety fused to the C-terminus of the masking moiety.

In some embodiments, the two cytokine moieties are separately fused to the carrier moiety through cleavable peptide linkers, wherein said cleavable linker comprises 4, 5, 6, 7, 8, 9, or 10 amino acids, optionally comprising SEQ ID NO: 77.

In some embodiments, the two cytokine moieties are separately fused to the carrier moiety directly or via non-cleavable peptide linkers and two masking moieties are separately fused to the two cytokine moieties directly or via non-cleavable or cleavable peptide linkers.

In some embodiments, two cytokine moieties are separately fused to the carrier moiety directly or via non-cleavable peptide linkers and one masking moiety is fused to one of the two cytokine moieties directly or via a non-cleavable or a cleavable peptide linker. In some embodiments, two masking moieties are separately fused to the carrier moiety directly or via non-cleavable peptide linkers and two cytokine moieties are separately fused to the two masking moieties directly or via non-cleavable peptide linkers.

In some embodiments, the cytokine moiety is fused to the carrier moiety directly or via a non-cleavable peptide linker and the masking moiety is fused to the carrier moiety directly or via a non-cleavable peptide linker, and a second cytokine moiety is fused to the C-terminus of the masking moiety directly or via a non-cleavable peptide linker.

In some embodiments, the carrier moiety comprises 1) an antibody or antigen-binding fragment thereof, or 2) an antibody Fc domain and two antigen-binding moieties, which are fused directly or via a non-cleavable peptide linker to the N-terminus or the C-terminus of the Fc domain. In some embodiments, the antibody or antigen-binding fragment thereof binds to an antigen expressed on the surface of an immune cell. In some embodiments, the immune cell is selected from an NK cell, a T cell, a B cell, and a macrophage and expresses a cell surface receptor for the cytokine moiety. In some embodiments, the Fc domain optionally comprises knobs-into-holes mutations. In some embodiments, the Fc domain or the Fc domain of the antibody optionally comprises RF mutations, wherein the RF mutations reduce or eliminate binding of the Fc domain to a protein A affinity resin. In some embodiments, the RF mutation is selected from H371R/Y372F (numbering according to SEQ ID NO: 107) or H453R/Y454F (numbering according to SEQ ID NO: 112).

In some embodiments, the carrier moiety comprises an antibody or antigen-binding fragment thereof that binds to an antigen selected from IL-1 receptor accessory protein (IL1RAP), IL-1 receptor (IL-1RI), a human IL-3 receptor, IL-4 receptor α chain (IL-4Rα), IL-5 receptor α chain (IL-5Rα), IL-6 receptor α chain (IL-6Rα), a human IL-9 receptor, a human IL-13 receptor, a human IL-17 receptor, a human IL-23 receptor, a human IL-31 receptor, a human IL-33 receptor, a receptor for thymic stromal lymphopoietin (TSLP), CD20, CD25, BCMA, CD40, CD80, CD86, Trem-1, CSF-1R, OX40, 4-1BB, TNF-alpha receptor 1 (TNFR-1), TNF-alpha receptor 1 (TNFR-2), a receptor for B lymphocyte stimulator (BLyS), mucosal addressin cell adhesion molecule 1 (MAdCAM-1), and an Interferon-alpha receptor.

In some embodiments, the carrier moiety comprises an antibody or antigen-binding fragment that comprises the same heavy and light chain complementarity-determining regions (CDRs), the same heavy and light variable domains, or the same heavy and light chains, as an antibody selected from canakinumab, adalimumab, CDP-571, infliximab, rontalizumab, sifalimumab, olokizumab (CDP6038), elsilimomab, BMS-945429 (ALD518), sirukumab (CNTO 136), levilimab (BCD-089), siltuximab, secukinumab, ixekizumab, ustekinuma, guselkumab, and tildrakizumab.

In some embodiments, the carrier moiety comprises an anti-IL-4 receptor α chain (IL-4Rα) antibody or an antigen-binding fragment thereof, which comprises light chain CDRs derived from SEQ ID NO: 11 and heavy chain CDRs derived from SEQ ID NO: 12; or a light chain variable domain with an amino acid sequence of SEQ ID NO: 13 or at least 95% identical thereto, and a heavy chain variable domain with an amino acid sequence of SEQ ID NO: 14 or at least 95% identical thereto.

In some embodiments, the carrier moiety comprises an anti-IL-5 receptor α chain (IL-5Rα) antibody or an antigen-binding fragment thereof, which comprises a light chain variable domain with an amino acid sequence of SEQ ID NO: 15 or at least 95% identical thereto, and a heavy chain variable domain with an amino acid sequence of SEQ ID NO: 16 or at least 95% identical thereto.

In some embodiments, the carrier moiety comprises an anti-IL-6 receptor α chain (IL-6Rα) antibody or a binding fragment thereof, which comprises a light chain variable domain with an amino acid sequence of SEQ ID NO: 17 or at least 95% identical thereto, and a heavy chain variable domain with an amino acid sequence of SEQ ID NO: 18 or at least 95% identical thereto; or a light chain variable domain with an amino acid sequence of SEQ ID NO: 19 or at least 95% identical thereto, and a heavy chain variable domain with an amino acid sequence of SEQ ID NO: 19 or at least 95% identical thereto.

In some embodiments, the carrier moiety comprises an anti-Trem-1 antibody or a fragment thereof, which comprises a light chain variable domain with an amino acid sequence of SEQ ID NO: 21 or at least 95% identical thereto, and a heavy chain variable domain with an amino acid sequence of SEQ ID NO: 22 or at least 95% identical thereto.

In some embodiments, the carrier moiety comprises an anti-CD86 antibody or a fragment thereof, which comprises a light chain variable domain with an amino acid sequence of SEQ ID NO: 23 or at least 95% identical thereto, and a heavy chain variable domain with an amino acid sequence of SEQ ID NO: 24 or at least 95% identical thereto.

In some embodiments, the carrier moiety comprises an extracellular domain of CTLA-4 (CTLA-4-ECD) or a functional analog thereof, which comprises an amino acid sequence of SEQ ID NO: 25 or 61 or at least 95% identical as that of SEQ ID NO: 25 or 61.

In some embodiments, the carrier moiety comprises an anti-interferon alpha receptor 1 (IFNRA-1) antibody or antigen-binding fragment thereof, which comprises a light chain variable domain with an amino acid sequence of SEQ ID NO: 52 or at least 95% identical as that of SEQ ID NO: 52, and a heavy chain variable domain with an amino acid sequence of SEQ ID NO: 51 or at least 95% identical as that of SEQ ID NO: 51.

In some embodiments, the carrier moiety comprises an anti-CD86 antibody or a fragment thereof, which comprises a light chain variable domain with an amino acid sequence of SEQ ID NO: 23 or at least 95% identical thereto, and a heavy chain variable domain with an amino acid sequence of SEQ ID NO: 24 or at least 95% identical thereto.

In some embodiments, the peptide linker that is cleavable by one or more proteases located at a site of inflammation or an autoimmune disease, optionally selected comprising a substrate sequence of urokinase-type plasminogen activator (uPA), matrix metallopeptidase (MT1-MMP), matrix metallopeptidase 2 (MMP2), MMP3, MMP9, matriptase, legumain, plasmin, TMPRSS-3/4, cathepsin, caspase, human neutrophil elastase, beta-secretase, or PSA, or (i) both uPA and MMP2, (ii) both uPA and MMP9, or (iii) matriptase, MMP2 and MMP9.

In some embodiments, the cleavable peptide linker is cleavable by one or more proteases located at a site of inflammation or an autoimmune disease.

In some embodiments, the non-cleavable peptide linker comprises an amino acid sequence selected from SEQ ID NOs: 95-99.

In some embodiments, the masking moiety inhibits the binding of the cytokine moiety to its receptor on the surface of a cell.

In some embodiments, the masking moiety comprises an extracellular domain of IL-10 receptor α chain (IL-10Rα-ECD), an analog of IL-10Rα-ECD, or an antibody against human IL-10 or a binding fragment thereof, or SEQ ID NO: 4, 5, or 6, or an amino acid sequence that is at least 95% identical thereto.

In some embodiments, the masking moiety comprises an extracellular domain of TGF-β Receptor II (TGFRII-ECD), an analog of TGFRII-ECD, or an antibody against human TGF-β or a binding fragment thereof.

In particular embodiments, the masking moiety comprises an extracellular domain of TGF-β Receptor II (TGFRII-ECD), an analog of TGFRII-ECD, or an antibody against human TGF-β or a binding fragment thereof, SEQ ID NO: 10, or an amino acid sequence that is at least 95% identical thereto, or a scFv that binds to human TGF-β, optionally wherein the scFv comprises a VH domain with an amino acid sequence of SEQ ID NO: 9 or at least 95% identical thereto, and a VL domain with an amino acid sequence of SEQ ID NO: 8 or at least 95% identical thereto.

In particular embodiments, the prodrug comprises two identical light chains and a first heavy chain polypeptide chain and a second heavy chain polypeptide chain, wherein the light chain comprises SEQ ID NO: 26 or an amino acid sequence at least 95% identical thereto the first heavy chain polypeptide chain comprises SEQ ID NO: 27 or 28, or an amino acid sequence at least 95% identical thereto, and the second heavy chain polypeptide chain comprises SEQ ID NO: 29 or 30, or an amino acid sequence at least 95% identical thereto.

In particular embodiments, the prodrug comprises two identical light chains and two identical heavy chains; wherein the light chain comprises SEQ ID NO: 26 or an amino acid sequence at least 95% identical thereto, and the heavy chain comprises SEQ ID NO: 31, 32, 33, 34, or 100 or an amino acid sequence at least 95% identical thereto.

In particular embodiments, the prodrug comprises two identical light chains and a first heavy chain polypeptide chain and a second heavy chain polypeptide chain; wherein the light chain comprises SEQ ID NO: 35 or an amino acid sequence at least 95% identical thereto the first heavy chain polypeptide chain comprises SEQ ID NO: 36 or an amino acid sequence at least 95% identical thereto, and the second heavy chain polypeptide chain comprises SEQ ID NO: 37 or 38, or an amino acid sequence at least 95% identical there.

In particular embodiments, the prodrug comprises two identical light chains and two identical heavy chains, wherein the light chain comprises SEQ ID NO: 35 or an amino acid sequence at least 95% identical thereto, and the heavy chain comprises SEQ ID NO: 39, 40, 41 or 42, or an amino acid sequence at least 95% identical thereto.

In particular embodiments, the prodrug comprises two identical light chains and a first heavy chain polypeptide chain and a second heavy chain polypeptide chain, wherein the light chain comprises SEQ ID NO: 43 or an amino acid sequence at least 95% identical thereto SEQ ID NO: 43, the first heavy chain polypeptide chain comprises SEQ ID NO: 44 or an amino acid sequence at least 95% identical thereto, and the second heavy chain polypeptide chain comprises SEQ ID NO: 45 or 46, or an amino acid sequence at least 95% identical thereto.

In particular embodiments, the prodrug comprises two identical light chains and two identical heavy chains, wherein the light chain comprises SEQ ID NO: 43 or said at least 95% identical thereto, and the heavy chain comprises SEQ ID NO: 47, 48, 49 or 50, or an amino acid sequence at least 95% identical thereto.

In particular embodiments, the prodrug comprises two identical light chains and a first heavy chain polypeptide chain and a second heavy chain polypeptide chain, the light chain comprises SEQ ID NO: 53 or an amino acid sequence at least 95% identical thereto, the first heavy chain polypeptide chain comprises SEQ ID NO: 54 or an amino acid sequence at least 95% identical thereto, and the second heavy chain polypeptide chain comprises SEQ ID NO: 55 or 56, or an amino acid sequence at least 95% identical thereto.

In particular embodiments, the prodrug comprises two identical light chains and two identical heavy chains, wherein the light chain comprises SEQ ID NO: 53 or an amino acid sequence at least 95% identical thereto, and the heavy chain comprises SEQ ID NO: 57, 58, 59 or 60, or an amino acid sequence at least 95% identical thereto.

In particular embodiments, the prodrug comprises two identical polypeptide chains comprising an amino acid sequence selected from SEQ ID NOs: 66, 67, 68, 69, 101-106 or at least 95% identical thereto.

In particular embodiments, the prodrug comprises a first polypeptide chain and a second polypeptide chain which form a heterodimer, wherein the first polypeptide chain comprises SEQ ID NO: 63 or 107, or an amino acid sequence at least 95% identical thereto, and the second polypeptide chain comprises SEQ ID NO: 64, 65, 105, or 106, or an amino acid sequence at least 95% identical thereto.

In particular embodiments, the prodrug comprises a first polypeptide chain and a second polypeptide chain which form a heterodimer; wherein the first polypeptide chain comprises SEQ ID NO: 107 or an amino acid sequence at least 95% identical thereto, and the second polypeptide chain comprises SEQ ID NO:105 or 106, or an amino acid sequence at least 95% identical thereto.

In particular embodiments, the prodrug comprises two identical light chains and two identical heavy chains, wherein the light chain comprises SEQ ID NO: 108 or an amino acid sequence at least 95% identical thereto SEQ ID NO: 108, and the heavy chain comprises SEQ ID NO: 109, 110, 111, or 113, or an amino acid sequence at least 95% identical thereto.

In particular embodiments, the prodrug comprises two identical light chains, a first heavy chain polypeptide chain, and a second heavy chain polypeptide chain, wherein the light chain comprises SEQ ID NO: 108 or an amino acid sequence at least 95% identical thereto, the first heavy chain polypeptide chain comprises SEQ ID NO: 110, or 111, or an amino acid sequence at least 95% identical thereto, and the second heavy chain polypeptide chain comprises SEQ ID NO: 112, or an amino acid sequence at least 95% identical thereto.

In particular embodiments, the prodrug comprises two identical light chains and two identical heavy chains, wherein the light chain comprises SEQ ID NO: 116, or an amino acid sequence at least 95% identical thereto, and the heavy chain polypeptide chain comprises SEQ ID NO: 114 or 115, or an amino acid sequence at least 95% identical thereto.

In some embodiments, the prodrug has a higher activity modulating an immune cell which expresses both the antigen targeted by the carrier moiety and a receptor for IL-10 or TGF-β than an immune cell which does not express both or either of the antigen and the cytokine receptor.

In other aspects, the present disclosure also provides a prodrug comprising pharmaceutical composition and a pharmaceutically acceptable excipient; a polynucleotide or polynucleotides encoding the prodrug; an expression vector or vectors comprising the polynucleotide or polynucleotides; and a host cell comprising the vector(s), wherein the host cell may be a prokaryotic cell or an eukaryotic cell such as a mammalian cell. In some embodiments, the mammalian host cell has the gene or genes encoding uPA, MMP3, MMP-2 and/or MMP-9 knocked out (e.g., containing null mutations of one or more of these genes).

In some embodiments, the present disclosure also provides a method of making the prodrug, comprising culturing the host cell under conditions that allow expression of the prodrug, wherein the host cell is a mammalian cell, and isolating the prodrug.

The present disclosure also provides a method of treating an autoimmune disease or inflammatory condition in a patient in need thereof comprising administering to the patient a therapeutically effective amount of the prodrug or pharmaceutical composition of the present disclosure. The patient may have, for example, asthma, atopic dermatitis, Type I diabetes, diabetic ulcers, allergy, psoriasis, rheumatoid arthritis, multiple sclerosis, osteoarthritis, graft vs host disease (GvHD), lupus nephritis, systemic lupus erythematosus (SLE), Alzheimer's disease, a neuron degeneration disease, an inflammatory bowel disease, ulcerative colitis, Crohn's disease NASH, atherosclerosis, and systemic sclerosis.

Also provided herein are a prodrugs or pharmaceutical compositions for use in treating an autoimmune disease or inflammatory condition in a patient in need thereof; use of a prodrug for the manufacture of a medicament for treating an autoimmune disease or inflammatory condition in a patient in need thereof; and articles of manufacture (e.g., kits) comprising one or more dosing units of the present prodrug. In some embodiments, the present prodrug can be further combined with another therapeutic, such as a pharmaceutical composition comprising an IL-2 mutein, an antagonist of TNFα, an antagonist of IL-12, an antagonist of IL-17 or its receptor, an antagonist of IL-23 or its receptor, an antagonist of IL-6 or its receptor, an antagonist of IL-5 or its receptor, an antagonist of IL-4 or its receptor, an antagonist of IL-1β or its receptor, an antagonist of interferon alpha receptor-1 (INFAR-1), an antagonist of CD40, an antagonist of CD80, or an antagonist of CD86.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE DRAWINGS

FIGS. 1A-1C illustrate cytokine prodrugs or chimeric molecules which comprise a carrier moiety, a masking moiety, and a cytokine moiety. FIG. 1A shows a cytokine prodrug comprising one cytokine moiety comprising a cytokine agonist polypeptide, a carrier moiety, and one masking moiety. The prodrug comprises two antigen-binding moieties that are separately fused to the N-termini of the Fc domain (i.e., on the two different Fc polypeptide chains of the Fc domain) optionally through non-cleavable peptide linkers to form the carrier moiety. The antigen-binding moieties may be selected from a CTLA-4 extracellular domain (CTLA-4-ECD), a TNFα receptor-2 extracellular domain (TNFAR-2-ECD), and peptide M3. Both the masking moiety and the cytokine moiety are separately fused to the C-termini of the Fc domain optionally through non-cleavable peptide linkers. The Fc domain contains knobs-into-holes mutations. FIG. 1B shows a cytokine prodrug comprising one cytokine moiety comprising a cytokine agonist polypeptide, a carrier moiety, and one masking moiety. The prodrug comprises two antigen-binding moieties that are separately fused to the C-termini of the Fc domain directly or optionally through non-cleavable peptide linkers to form the carrier moiety. For example, the antigen-binding moieties may be selected from CTLA-4-ECD, TNFAR-2-ECD, and peptide M3. Both the masking moiety and the cytokine moiety are separately fused to the N-termini of the Fc domain, which contains knobs-into-holes mutations, optionally through non-cleavable peptide linkers. FIG. 1C shows a cytokine prodrug comprising one cytokine moiety comprising a cytokine agonist polypeptide, a carrier moiety, and one masking moiety. The carrier moiety comprises an antibody. The masking moiety and the cytokine moiety are separately fused to the C-termini of the heavy chains (on different heavy chains) optionally through non-cleavable peptide linkers.

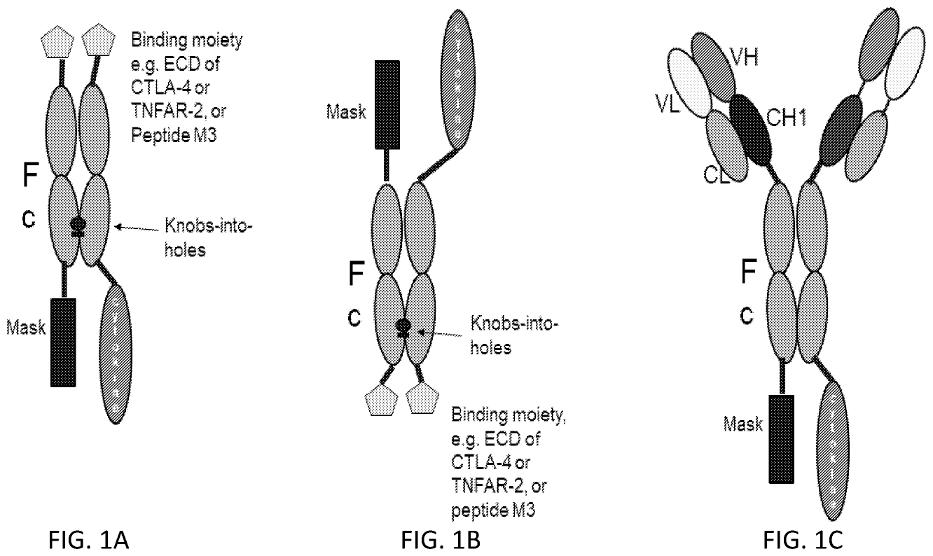

FIGS. 2A-2D illustrate cytokine prodrugs or chimeric molecules which comprise a carrier moiety, at least one masking moiety and two cytokine moieties. FIG. 2A shows a cytokine prodrug comprising two IL-10 agonist polypeptides (cytokine moieties), a carrier moiety, and one masking moiety. The prodrug comprises two antigen-binding moieties that are separately fused to the N-termini of the Fc domain optionally through non-cleavable peptide linkers to form the carrier moiety. The Fc domain contains knobs-into-holes mutations. Two IL-10 agonist polypeptides are linked to each other optionally through a non-cleavable peptide linker and are fused to the C-terminus of one of the Fc polypeptide chains of the Fc domain portion of the carrier moiety. The IL-10 agonist polypeptide that is proximal to the Fc domain is fused to the C-terminus of one of the Fc polypeptide chains of the Fc domain optionally through a non-cleavable peptide linker. The masking moiety is fused to the C-terminus of the other Fc polypeptide chain of the Fc domain. FIG. 2B shows a cytokine prodrug comprising two cytokine moieties, a carrier moiety, and two masking moieties. The prodrug comprises two antigen-binding moieties that are fused to the N-termini of the Fc domain optionally through non-cleavable peptide linkers to form the carrier moiety. The Fc domain contains knobs-into-holes mutations. Two IL-10 agonist polypeptides are linked to each other optionally through a non-cleavable peptide linker and are fused to the C-terminus of one of the Fc polypeptide chains of the Fc domain. The IL-10 agonist polypeptide that is proximal to the Fc domain is fused to the C-terminus of one of the Fc polypeptide chains of the Fc domain optionally through a non-cleavable peptide linker. The first masking moiety is fused to C-terminus of the other Fc polypeptide of the Fc domain optionally through a non-cleavable peptide linker, and the second masking moiety is fused to the C-terminus of the first masking moiety optionally through a peptide linker, which is optionally a cleavable peptide linker. FIG. 2C shows a cytokine prodrug comprising an antibody as the carrier moiety, two cytokine moieties, and one masking moiety. Two IL-10 agonist polypeptides are linked to each other optionally through a peptide linker and are fused to the C-terminus of one of the heavy chains of the antibody. The IL-10 agonist polypeptide that is proximal to the heavy chains of the antibody is fused to the C-terminus of one of the heavy chains of the antibody optionally through a non-cleavable peptide linker. The masking moiety is fused to the C-terminus of the other heavy chain of the antibody. FIG. 2D shows a cytokine prodrug comprising an antibody as the carrier moiety, two cytokine moieties, and two masking moieties. Two IL-10 agonist polypeptides are linked to each other through a peptide linker and are fused to the C-terminus of one of the heavy chains of the antibody. The IL-10 agonist polypeptide that is proximal to the heavy chains of the antibody is fused to the C-terminus of one of the heavy chains of the antibody optionally through a non-cleavable peptide linker. The first masking moiety is fused to the C-terminus of the other heavy chain of the antibody, and the second masking moiety is fused to the C-terminus of the first masking moiety optionally through a cleavable peptide linker.

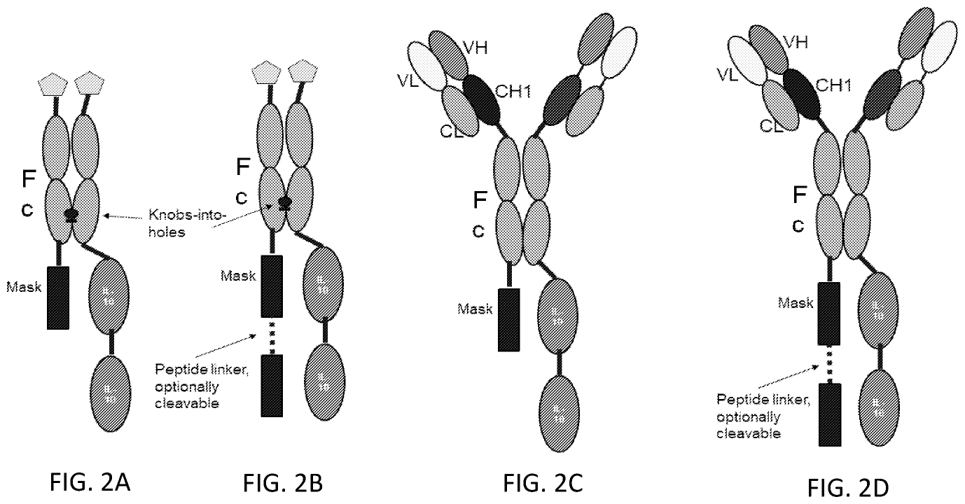

FIG. 3 shows a chimeric molecule comprising an antibody as the carrier moiety and two cytokine moieties. Two IL-10 agonist polypeptides are separately fused to the C-terminus of the heavy chains of the antibody through cleavable peptide linkers.

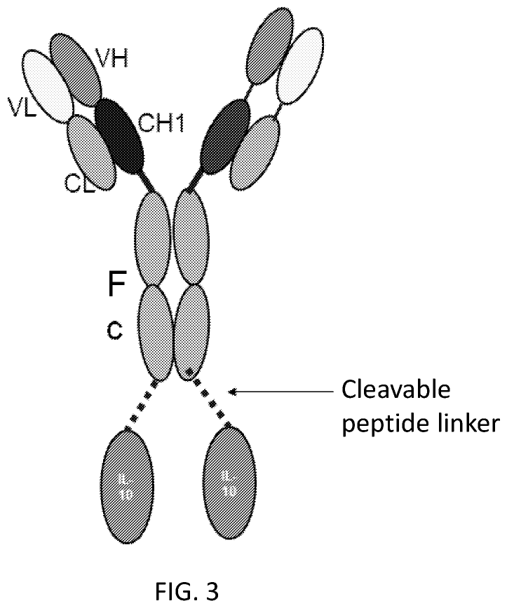

FIGS. 4A and 4B show cytokine prodrugs comprising an antibody as the carrier moiety, two cytokine moieties, and two masking moieties. FIG. 4A shows a cytokine prodrug which comprises an antibody as the carrier moiety, two IL-10 agonist polypeptides that are separately fused to the C-termini of the heavy chains of the antibody, and two masking moieties that are each fused to the C-terminus of the two IL-10 agonist polypeptides, optionally through non-cleavable peptide linkers. FIG. 4B shows a cytokine prodrug which comprises two IL-10 agonist polypeptides that are separately fused to the C-termini of the heavy chains of the antibody, and two masking moieties that are each fused to the C-terminus of the two IL-10 agonist polypeptides, optionally through a cleavable peptide linker.

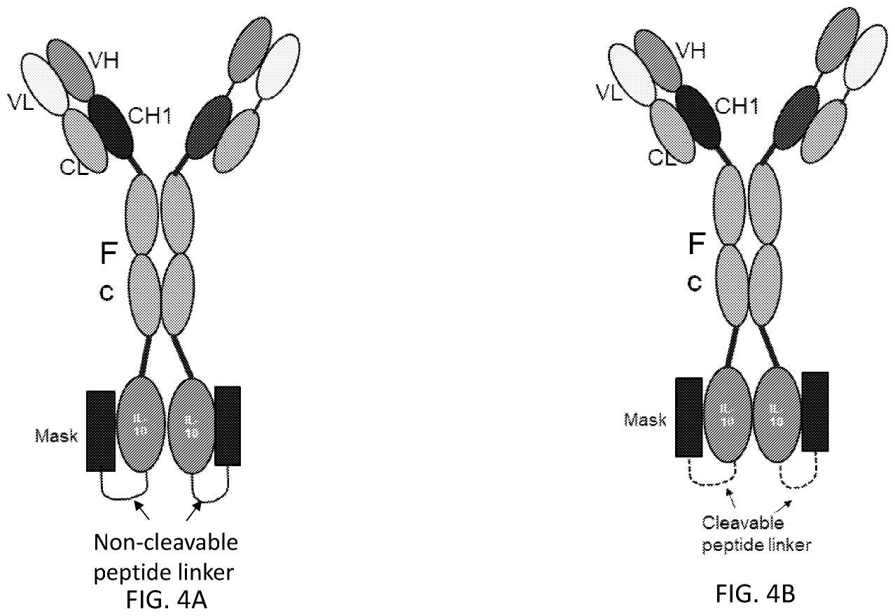

FIG. 5 shows a cytokine prodrug comprising an antibody as the carrier moiety, two IL-10 agonist polypeptides, and two masking moieties; wherein the cytokine moieties are fused to the C-termini of the heavy chains of the antibody, and the IL-10 agonist polypeptides are fused to the C-termini of the masking moieties. The two masking moieties are separately fused to the C-termini of the antibody and the two IL-10 agonist polypeptides are each fused to the C-terminus of the two masking moieties through a non-cleavable peptide linker.

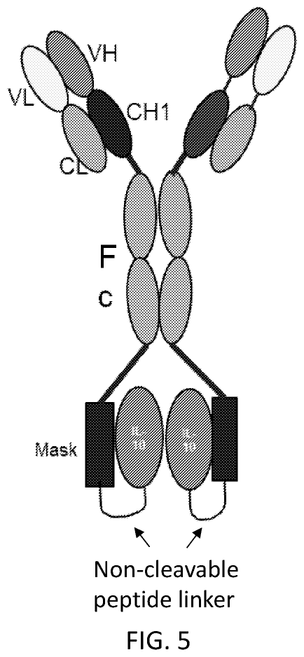

FIG. 6A shows the amino acid sequence information for IL-10 prodrugs which comprise CTLA-4 extracellular domains (ECDs) as antigen-binding moieties. Prodrugs IL-10-B, IL-10-C, IL-10-D, IL-10-E and IL-10-F are homodimers, while IL-10-G, IL-10-H, IL-10-P, and IL-10-Q are heterodimers. The homodimer prodrugs comprise two IL-10 moieties and two masking moieties. The heterodimer prodrugs comprise two IL-10 moieties and one masking moiety.

FIG. 6B-6G shows the schematic drawings of the IL-10 prodrugs IL-10-C, IL-10-D, IL-10-F, IL-10-P, IL-10-Q, and IL-10-H, which are prodrugs that comprise a cytokine moiety comprising an IL-10 agonist polypeptide, a carrier moiety, and a masking moiety. FIG. 6B shows an IL-10 prodrug (IL-10-C/JR11.48.3) comprising two CTLA4-ECD moieties separately fused to the N-termini of an IgG1 Fc domain to form the carrier moiety. Two IL-10 agonist polypeptides are separately fused to the C-termini of the IgG1 Fc domain via non-cleavable peptide linkers. Two single chain variable fragment (scFv) masking moieties, each comprising the amino acid sequence of SEQ ID NO: 5, are fused to each IL-10 agonist polypeptide via the IL-10 C-terminus through non-cleavable peptide linkers. FIG. 6C shows an IL-10 prodrug (IL-10-D/JR11.48.4) comprising two CTLA4-ECD moieties directly but separately fused to the N-termini of an IgG1 Fc domain to form the carrier moiety. Two IL-10 agonist polypeptides are separately fused to the C-termini of the IgG1 Fc domain via non-cleavable peptide linkers. Two single chain variable fragment (scFv) masking moieties each comprising the amino acid sequence of SEQ ID NO: 6 are fused to each IL-10 agonist polypeptide via the IL-10 C-terminus through non-cleavable peptide linkers. FIG. 6D shows an IL-10 prodrug (IL-10-F/JR.11.49.1) comprising two CTLA4-ECD moieties separately fused to the N-termini of an IgG1 Fc domain to form the carrier moiety. Two scFv masking moieties, each comprising the amino acid sequence of SEQ ID NO: 6, are separately fused to the C-termini of the IgG1 Fc domain. Two IL-10 agonist polypeptides are each fused to the C-terminus of the two scFv masking moieties via a non-cleavable peptide linker. FIG. 6E shows an IL-10 prodrug (IL-10-P/JR11.49.2) comprising two CTLA4-ECD moieties separately fused to the N-termini of an IgG1 Fc domain to form the carrier moiety. Two IL-10 agonist polypeptides are separately fused to the C-termini of the IgG1 Fc domain via non-cleavable peptide linkers. A single chain variable fragment (scFv) masking moiety comprising the amino acid sequence of SEQ ID NO: 5 is fused to one of the two IL-10 agonist polypeptides via the IL-10 C-terminus through a non-cleavable peptide linker. The Fc domain comprises RF mutations (H371R/Y372F; numbering according to SEQ ID NO: 107). FIG. 6F shows an IL-10 prodrug (IL-10-P/JR11.49.2) comprising two CTLA4-ECD moieties separately fused to the N-termini of an IgG1 Fc domain to form the carrier moiety. Two IL-10 agonist polypeptides are separately fused to the C-termini of the IgG1 Fc domain via non-cleavable peptide linkers. A single chain variable fragment (scFv) masking moiety comprising the amino acid sequence of SEQ ID NO: 6 is fused to one of the two IL-10 agonist polypeptides via the IL-10 C-terminus through a non-cleavable peptide linker. The Fc domain comprises RF mutations (H371R/Y372F; numbering according to SEQ ID NO: 107). FIG. 6G shows an IL-10 prodrug (IL-10-P/JR11.49.2) comprising two CTLA4-ECD moieties separately fused to the N-termini of an IgG1 Fc domain to form the carrier moiety. One IL-10 agonist polypeptide is fused to the C-terminus of one Fc polypeptide chain of the IgG1 Fc domain via a non-cleavable peptide linker. A single chain variable fragment (scFv) masking moiety comprising the amino acid sequence of SEQ ID NO: 6 is fused to the other Fc polypeptide chain of the IgG1 Fc domain via a non-cleavable peptide linker. A second IL-10 agonist polypeptide is fused to the C-terminus of the scFv masking moiety through a non-cleavable peptide linker. The Fc domain comprises RF mutations (H371R/Y372F; numbering according to SEQ ID NO: 107).

FIG. 6H shows the reporter assay results of the IL-10 prodrug molecules. The activities of the negative control (CTLA-4 ECD-Fc) and the positive control (IL-10) were also included in the assay.

FIG. 6I shows the prodrug activities as compared to IL-10.

FIG. 7A shows the amino acid sequence information for IL-10 prodrugs which comprise an antibody against Trem-1 as the carrier. Molecules IL-10-I, and IL-10-J are homodimers, while IL-10-K, and IL-10-O are heterodimers. The homodimer prodrug molecules comprise two IL-10 moieties and two masking moieties. The heterodimers comprise two IL-10 moieties and one masking moiety.

FIGS. 7B-7E shows the schematic drawings of the IL-10 prodrugs. IL-10-I (FIG. 7B) and IL-10-J (FIG. 7C), both of which comprise identical heavy chain polypeptides. Both prodrugs comprise an antibody carrier moiety, two cytokine moieties, and two masking moieties. Prodrugs in FIG. 7B IL-10-I (FIG. 7B) and IL-10-K (FIG. 7D) comprise a carrier moiety, two cytokine moieties and one masking moiety. IL-10-I (FIG. 7B) and IL-10-K (FIG. 7D) comprise cleavable peptide linkers. FIG. 7B shows a prodrug comprising an antibody as a carrier moiety, two IL-10 agonist polypeptides separately fused to the C-termini of the heavy chains of the antibody via non-cleavable peptide linkers. Two scFv masking moieties, each comprising an amino acid sequence of SEQ ID NO: 5, are separately fused to the C-terminus of the two IL-10 agonist polypeptides via cleavable peptide linkers. FIG. 7C shows a prodrug comprising an antibody as a carrier moiety with two scFv masking moieties, each comprising an amino acid sequence of SEQ ID NO: 5, that are separately fused to the C-termini of the heavy chains of the antibody via non-cleavable peptide linkers. Two IL-10 agonist polypeptides are separately fused to the C-termini of the scFv masking moieties via non-cleavable peptide linkers. FIG. 7D shows a prodrug comprising an antibody as a carrier moiety, wherein the antibody Fc domain comprises RF mutations H453R/Y454F (numbering according to SEQ ID NO: 112). Two IL-10 agonist polypeptides are separately fused to the C-termini of the heavy chains of the antibody via non-cleavable peptide linkers. A scFv masking moiety comprising an amino acid sequence of SEQ ID NO: 5 is separately fused to the C-termini of one of the two IL-10 agonist polypeptides via a cleavable peptide linker. FIG. 7E shows a prodrug comprising an antibody as a carrier moiety, wherein the antibody Fc domain comprises RF mutations H453R/Y454F (numbering according to SEQ ID NO: 112). An IL-10 agonist polypeptide is fused to the C-terminus of the heavy chains of the antibody via non-cleavable peptide linkers. A scFv masking moiety comprising an amino acid sequence of SEQ ID NO: 5 is fused to the C-terminus of one of the heavy chains of the antibody via a non-cleavable peptide linker. A second IL-10 agonist polypeptide is fused to the C-terminus of the masking moiety via non-cleavable peptide linkers.

FIG. 7F shows the reporter assay results of the IL-10 prodrug molecules.

FIG. 7G shows the IL-10 prodrug activities as compared to IL-10.

FIG. 8A shows the SEC-HPLC purity of the purified IL-10-J sample.

FIG. 8B shows the reporter assay activity of the purified IL-10-J sample.

### DETAILED DESCRIPTION OF THE INVENTION

As used herein and in the appended claims, the singular forms “a,” “or,” and “the” include plural referents unless the context clearly dictates otherwise. Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z.”

The term “antigen-binding moiety” refers to a polypeptide or a set of interacting polypeptides that specifically bind to an antigen, and includes, but is not limited to, an antibody (e.g., a monoclonal antibody, polyclonal antibody, a multi-specific antibody, a dual specific or bispecific antibody, an anti-idiotypic antibody, or a bifunctional hybrid antibody) or an antigen-binding fragment thereof (e.g., a Fab, a Fab′, a F(ab′)₂, a Fv, a disulfide linked Fv, a scFv, a single domain antibody (dAb), or a diabody), a single chain antibody, and an Fc-containing polypeptide such as an immunoadhesin. In some embodiments, the antibody may be of any heavy chain isotype (e.g., IgG, IgA, IgM, IgE, or IgD) or subtype (e.g., IgG₁, IgG₂, IgG₃, or IgG₄). In some embodiments, the antibody may be of any light chain isotype (e.g., kappa or lambda). The antibody may be human, non-human (e.g., from mouse, rat, rabbit, goat, or another non-human animal), chimeric (e.g., with a non-human variable region and a human constant region), or humanized (e.g., with non-human CDRs and human framework and constant regions). In some embodiments, the antibody is a derivatized antibody.

The terms “cytokine agonist polypeptide” or “cytokine moiety” refers to a wildtype cytokine, or an analog thereof. An analog of a wildtype cytokine has the same biological specificity (e.g., binding to the same receptor(s) and activating the same target cells) as the wildtype cytokine, although the activity level of the analog may be different from that of the wildtype cytokine. The analog may be, for example, a mutein (i.e., mutated polypeptide) of the wildtype cytokine, and may comprise at least one, at least two, at least three, at least four, at least five, at least six, at least seven, at least eight, at least nine, or at least ten mutations relative to the wildtype cytokine.

The term “cytokine mask” or “masking moiety” refers to a moiety (e.g., a polypeptide) that binds to a cytokine, thereby inhibiting the cytokine from binding to its receptor on the surface of a target cell and/or exerting its biological functions while being bound by the mask. Examples of a cytokine mask include, without limitations, a polypeptide derived from an extracellular domain of the cytokine's natural receptor that makes contact with the cytokine.

The term “effective amount” or “therapeutically effective amount” refers to an amount of a compound or composition sufficient to treat a specified disorder, condition, or disease, such as ameliorate, palliate, lessen, and/or delay one or more of its symptoms.

The term “functional analog” refers to a molecule that has the same biological specificity (e.g., binding to the same ligand) and/or activity (e.g., activating or inhibiting a target cell) as a reference molecule.

The term “fused” or “fusion” in reference to two polypeptide sequences refers to the joining of the two polypeptide sequences through a backbone peptide bond. Two polypeptides may be fused directly or through a peptide linker that is one or more amino acids long. A fusion polypeptide may be made by recombinant technology from a coding sequence containing the respective coding sequences for the two fusion partners, with or without a coding sequence for a peptide linker in between. In some embodiments, fusion encompasses chemical conjugation.

The term “pharmaceutically acceptable excipient” when used to refer to an ingredient in a composition means that the excipient is suitable for administration to a treatment subject, including a human subject, without undue deleterious side effects to the subject and without affecting the biological activity of the active pharmaceutical ingredient (API).

The term “subject” refers to a mammal and includes, but is not limited to, a human, a pet (e.g., a canine or a feline), a farm animal (e.g., cattle or horse), a rodent, or a primate.

As used herein, “treatment” or “treating” is an approach for obtaining beneficial or desired clinical results. Beneficial or desired clinical results include, but are not limited to, one or more of the following: alleviating one or more symptoms resulting from a disease, diminishing the extent of a disease, ameliorating a disease state, stabilizing a disease (e.g., preventing or delaying the worsening or progression of the disease), preventing or delaying the spread (e.g., metastasis) of a disease, preventing or delaying the recurrence of a disease, providing partial or total remission of a disease, decreasing the dose of one or more other medications required to treat a disease, increasing the patient's quality of life, and/or prolonging survival. The methods of the present disclosure contemplate any one or more of these aspects of treatment.

It is to be understood that one, some or all of the properties of the various embodiments described herein may be combined to form other embodiments of the present invention. The section headings used herein are for organizational purposes only and are not to be construed as limiting the subject matter described thereunder.

### I. IL-10 and TGF-β Prodrugs

The present disclosure provides chimeric molecules or cytokine prodrugs that are useful to treat an autoimmune disease or an inflammation condition of a patient. In some embodiments, the cytokine prodrugs comprise a cytokine moiety, a masking moiety, a carrier moiety, and one or more peptide linkers. The terms “chimeric molecules” and “cytokine prodrugs” or “prodrugs” may be used interchangeably herein, with the exception that chimeric molecules may also include prodrug molecules following activation (i.e., after cleavage of one or more masking moieties).

In some embodiments, the cytokine moiety comprises an IL-10 agonist polypeptide or a TGF-β agonist polypeptide. In some embodiments, the carrier moiety comprises an Fc domain. In some embodiments, the carrier moiety comprises an antibody, or an antibody Fc domain with two antigen-binding moieties. In some embodiments, the masking moiety comprises an extracellular domain of IL-10 receptor alpha chain (IL-10Rα-ECD), a scFv, or Fab which binds to human IL-10. In some embodiments, the masking moiety comprises an extracellular domain of TGF-β Receptor II (TGFRII-ECD), a scFv, or Fab which binds to TGF-β. In some embodiments, the prodrug comprises one or more peptide linkers, which can optionally be non-cleavable or cleavable.

A. Cytokine Moieties of the Prodrugs

In some embodiments, the cytokine moiety comprises an IL-10 agonist polypeptide or a TGF-β agonist polypeptide. In some embodiments, the IL-10 agonist polypeptide comprises an amino acid sequence of SEQ ID NO: 1 or 2 or at least 90% identical as that of SEQ ID NO: 1 or 2. In a particular embodiment, the IL-10 agonist polypeptide comprises a F111S mutation (numbering according to SEQ ID NO: 1). In another particular embodiment, the IL-10 agonist polypeptide comprises an amino acid sequence of SEQ ID NO: 3.

In some embodiments, the cytokine moiety comprises TGF-β and the masking moiety binds to human TGF-β1. In some embodiments, the cytokine comprises an amino acid sequence of SEQ ID NO: 7, or at least 90% identical to SEQ ID NO: 7. In some embodiments, the masking moiety comprises an amino acid sequence of SEQ ID NO: 10, or at least 90% identical to SEQ ID NO: 10. In some embodiments, the masking moiety comprises a VH domain with an amino acid sequence of SEQ ID NO: 9 or at least 90% identical to SEQ ID NO: 9, and a VL domain with an amino acid sequence of SEQ ID NO: 8 or at least 90% identical to SEQ ID NO: 8.

B. Masking Moieties of the Prodrugs

The masking moiety in the present prodrugs may comprise a peptide, an antibody, or antibody fragment that binds to the cytokine moiety in the prodrug, thereby masking the cytokine moiety and inhibiting its biological functions. In some embodiments, the masking moiety is operationally linked to the rest of the prodrug through a peptide linker (cleavable or non-cleavable).

By way of example, the prodrug comprises an IL-10 or TGF-β agonist polypeptide and the masking moiety comprises a peptide, or an antibody, or antibody fragment that binds IL-10 or TGF-β and interferes with the binding of the IL-10 or TGF-β to its cognate receptors. In some embodiments, the masking moiety reduces biological activities of the IL-10 or TGF-β moiety while masked.

In some embodiments, the masking moiety comprises an extracellular domain of IL-10 receptor α chain (IL-10Rα-ECD), an analog of IL-10Rα-ECD, or an antibody against human IL-10 or a binding fragment thereof. In some embodiments, the masking moiety comprises an amino acid sequence of SEQ ID NO: 4, 5, or 6, or at least 95% identical as SEQ ID NO: 4, 5, or 6.

In some embodiments, the masking moiety comprises a scFv or Fab which binds to human IL-10. In some embodiments, the masking moiety comprises a scFv with an amino acid sequence of SEQ ID NO: 4 or 5 or at least 90% identical to SEQ ID NO: 4 or 5. In some embodiments, the masking moiety binds to human IL-10. In some embodiments, the masking moiety optionally comprises the extracellular domain of IL-10, which comprises an amino acid sequence of SEQ ID NO: 6 or at least 90% identical as that of SEQ ID NO: 6.

In some embodiments, the masking moiety comprises an extracellular domain of TGF-β Receptor II (TGFRII-ECD), an analog of TGFRII-ECD, or an antibody against human TGF-β or a binding fragment thereof. In some embodiments, the masking moiety is a scFv which binds to human TGF-β, wherein the scFv comprises a VH domain with an amino acid sequence of SEQ ID NO: 9 or at least 95% identical to SEQ ID NO: 9, and a VL domain with an amino acid sequence of SEQ ID NO: 8 or at least 95% identical to SEQ ID NO: 8.

In some embodiments, the IL-10 masking moiety comprises a peptide identified through a peptide library screening.

C. Carrier Moieties of the Prodrugs

The carrier moieties of the present prodrugs may be an antigen-binding moiety, or a moiety that is not antigen-binding. The carrier moiety may improve the PK profiles, such as serum half-life, of the cytokine agonist polypeptide and may also target the cytokine agonist polypeptide to a target site in the body, such as a disease site.

1. Antigen-Binding Domains of Carrier Moieties

The carrier moiety comprises an antigen-binding domain and may be an antibody or an antigen-binding fragment thereof, or an immunoadhesin. In some embodiments, the antigen-binding carrier moiety is a full-length antibody with two heavy chains and two light chains, a Fab fragment, a Fab′ fragment, a F(ab′)₂ fragment, a Fv fragment, a disulfide linked Fv fragment, a single domain antibody, a nanobody, or a single-chain variable fragment (scFv). In some embodiments, the antigen-binding moiety is a bispecific antigen-binding moiety and can bind to two different antigens or two different epitopes on the same antigen. In some embodiments, the carrier moiety comprises two antigen-binding domains that share the same antigen specificity. The antigen-binding moiety may provide additional and potentially synergetic therapeutic efficacy to the cytokine agonist polypeptide.

The cytokine agonist polypeptide and its mask may be fused to the N-terminus or C-terminus of the light chain(s) and/or heavy chain(s) of the antigen-binding moiety. By way of example, the IL-10 or TGF-β agonist polypeptide and its mask may be fused to the antibody heavy chain or an antigen-binding fragment thereof or to the antibody light chain or an antigen-binding fragment thereof. In some embodiments, one terminus of the IL-10 or TGF-β agonist polypeptide is fused to the C-terminus of one or both heavy chains of an antibody, and the IL-10 mask is fused to the other terminus of the IL-10 or TGF-β agonist polypeptide through a non-cleavable peptide linker. In some embodiments, the IL-10 or TGF-β agonist polypeptide is fused to the C-terminus of one of the heavy chains of an antibody, and the IL-10 or TGF-β mask is fused to the C-terminus of the other heavy chain of the antibody through a non-cleavable peptide linker, wherein the two heavy chains contain mutations that allow the specific pairing of the two different heavy chains.

Strategies for forming heterodimers for Fc-fusion polypeptides or bispecific antibodies are well known (see, e.g., Spies et al., Mol Imm. (2015) 67(2)(A):95-106). For example, the two heavy chain polypeptides in the prodrug may form stable heterodimers through “knobs-into-holes” mutations. “Knobs-into-holes” mutations are made to promote the formation of the heterodimers of the antibody heavy chains and are commonly used to make bispecific antibodies (see, e.g., U.S. Pat. No. 8,642,745). For example, the Fc domain of the antibody may comprise a T366W mutation in the CH3 domain of the “knob chain” and T366S, L368A, and/or Y407V mutations in the CH3 domain of the “hole chain.” An additional interchain disulfide bridge between the CH3 domains can also be used, e.g., by introducing a Y349C mutation into the CH3 domain of the “knobs chain” and an E356C or S354C mutation into the CH3 domain of the “hole chain” (see, e.g., Merchant et al., Nature Biotech (1998) 16:677-81). In other embodiments, the antibody moiety may comprise Y349C and/or T366W mutations in one of the two CH3 domains, and E356C, T366S, L368A, and/or Y407V mutations in the other CH3 domain. In certain embodiments, the antibody moiety may comprise Y349C and/or T366W mutations in one of the two CH3 domains, and S354C (or E356C), T366S, L368A, and/or Y407V mutations in the other CH3 domain, with the additional Y349C mutation in one CH3 domain and the additional E356C or S354C mutation in the other CH3 domain, forming an interchain disulfide bridge (numbering always according to EU index of Kabat; Kabat et al., “Sequences of Proteins of Immunological Interest,” 5th ed., Public Health Service, National Institutes of Health, Bethesda, Md. (1991)). Other knobs-into-holes technologies, such as those described in EP1870459A1, can be used alternatively or additionally. Thus, another example of knobs-into-holes mutations for an antibody moiety is having R409D/K370E mutations in the CH3 domain of the “knob chain” and D399K/E357K mutations in the CH3 domain of the “hole chain” (EU numbering).

In some embodiments, the antigen-binding moiety in the prodrug is an antibody that comprises L234A and L235A (“LALA”) mutations in its Fc domain. The LALA mutations eliminate complement binding and fixation as well as Fcγ dependent ADCC (see, e.g., Hezareh et al., J. Virol. (2001) 75(24):12161-8). In further embodiments, the LALA mutations are present in the antibody moiety in addition to the knobs-into-holes mutations. In some embodiments, the antibody moiety in the prodrug further comprises G237A mutation in its Fc domain.

In some embodiments, the antigen-binding moiety is an antibody that comprises the M252Y/S254T/T256E (“YTE”) mutations in the Fc domain. The YTE mutations allow the simultaneous modulation of serum half-life, tissue distribution and activity of IgG₁ (see Dall'Acqua et al., J Biol Chem. (2006) 281:23514-24; and Robbie et al., Antimicrob Agents Chemother. (2013) 57(12):6147-53). In further embodiments, the YTE mutations are present in the antibody in addition to the knobs-into-holes mutations. In particular embodiments, the antibody has YTE, LALA and knobs-into-holes mutations or any combination thereof.

In some embodiments, the antibody or antigen-binding fragment thereof binds to a receptor of a cytokine (except the receptor for IL-10 or TGF-β) or a receptor for a chemokine. In some embodiments, the antibody or antigen-binding fragment binds to an antigen on an immune cell, for examples, IL-1 receptor accessory protein (IL1RAP), IL-1 receptor (IL-1RI), a human IL-3 receptor, IL-4 receptor α chain (IL-4Rα), IL-5 receptor α chain (IL-5Rα), IL-6 receptor α chain (IL-6Rα), a human IL-9 receptor, a human IL-13 receptor, a human IL-17 receptor, a human IL-23 receptor, a human IL-31 receptor, a human IL-33 receptor, a receptor for thymic stromal lymphopoietin (TSLP), CD20, CD25, BCMA, CD40, CD80, CD86, mucosal addressin cell adhesion molecule 1 (MAdCAM-1), triggering receptors expressed on myeloid cells-1 (Trem-1), Colony-stimulating factor 1 receptor (CSF-1R), OX40, 4-1BB, TNF-alpha receptor 1 (TNFR-1), TNF-alpha receptor 1 (TNFR-2), a receptor for B lymphocyte stimulator (BLyS), and an Interferon-alpha receptor. In some embodiments, the antibody binds to a cytokine (except IL-10 and TGF-β). In some embodiments, the antibody or antigen-binding fragment thereof binds to and neutralizes a biological activity of a cytokine selected from IL-1α, IL-1(3, IL-4, IL-5, IL-6, IL-12, IL-13, IL-17, IL-23, IL-31, IL-33, tumor necrosis factor alpha (TNFα), and interferon alpha (IFNα), interferon gamma (IFNγ). In other embodiments, the antibody or antigen-binding fragment thereof binds to and neutralizes a biological activity of a cytokine selected from IL-1α, IL-1(3, IL-4, IL-5, IL-6, 11-12, IL-13, IL-17, IL-23, IL-31, IL-33, TNFα, and interferon alpha (IFNα), interferon gamma (IFNγ) or binds to and neutralizes a chemokine.

In some embodiments, the antigen-binding moiety comprises the six complementarity determining regions (CDRs) of dupilumab, benralizumab, tocilizumab, sarilumab, canakinumab, adalimumab, CDP-571, infliximab, rontalizumab, sifalimumab, olokizumab (CDP6038), elsilimomab, BMS-945429 (ALD518), sirukumab (CNTO 136), levilimab (BCD-089), siltuximab, secukinumab, ixekizumab, ustekinuma, guselkumab and tildrakizumab.

A number of CDR delineations are known in the art and are encompassed herein. A person of skill in the art can readily determine a CDR for a given delineation based on the sequence of the heavy or light chain variable region. The “Kabat” CDRs are based on sequence variability and are the most commonly used (Kabat et al., Sequences of Proteins of Immunological Interest, 5th Ed. Public Health Service, National Institutes of Health, Bethesda, Md. (1991)). “Chothia” CDRs refer to the location of the structural loops (Chothia & Lesk, J. Mol. Biol. (1987) 196:901-917). The “AbM” CDRs represent a compromise between the Kabat CDRs and Chothia structural loops, and are used by Oxford Molecular's AbM antibody modeling software. The “Contact” CDRs are based on an analysis of the available complex crystal structures. The residues from each of these CDRs are noted below in Table 1, in reference to common antibody numbering schemes. Unless otherwise specified herein, amino acid numbers in antibodies refer to the Kabat numbering scheme as described in Kabat et al., supra, including when CDR delineations are made in reference to Kabat, Chothia, AbM, or Contact schemes. Using this numbering system, the actual linear amino acid sequence may contain fewer or additional amino acids corresponding to a shortening of, or insertion into, a framework region (FR) or CDR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

TABLE 1
CDR Delineations According to Various Schemes

| CDR | Kabat | AbM | Chothia | Contact |
|---|---|---|---|---|
| VL-CDR1 | L24-L34 | L24-L34 | L26-L32 | L30-L36 |
| VL-CDR2 | L50-L56 | L50-L56 | L50-L52 | L46-L55 |
| VL-CDR3 | L89-L97 | L89-L97 | L91-L96 | L89-L96 |
| VH-CDR1 | H31-H35B | H26-H35B | H26-H32 | H30-H35B |
| (Kabat nos.) | | | | |
| VH-CDR1 | H31-H35 | H26-H35 | H26-H32 | H30-H35 |
| (Chothia nos.) | | | | |
| VH-CDR2 | H50-H65 | H50-H58 | H53-H55 | H47-H58 |
| VH-CDR3 | H95-H102 | H95-H102 | H95-H101 | H93-H101 |

In some embodiments, the CDRs are “extended CDRs,” and encompass a region that begins or terminates according to a different scheme. For example, an extended CDR can be as follows: L24-L36, L26-L34, or L26-L36 (VL-CDR1); L46-L52, L46-L56, or L50-L55 (VL-CDR2); L91-L97 (VL-CDR3); H47-H55, H47-H65, H50-H55, H53-H58, or H53-H65 (VH-CDR2); and/or H93-H102 (VH-CDR3).

In some embodiments, the antigen-binding moiety comprises one, two, three, or four antigen-binding domains. For example, the antigen-binding moiety is bispecific and binds to two different antigens selected from the group consisting of IL-1 receptor accessory protein (IL1RAP), IL-1 receptor (IL-1RI), a human IL-3 receptor, IL-4 receptor α chain (IL-4Rα), IL-5 receptor α chain (IL-5Rα), IL-6 receptor α chain (IL-6Rα), a human IL-9 receptor, a human IL-13 receptor, a human IL-17 receptor, a human IL-23 receptor, a human IL-31 receptor, a human IL-33 receptor, a receptor for thymic stromal lymphopoietin (TSLP), CD20, CD25, BCMA, CD40, CD80, CD86, mucosal addressin cell adhesion molecule 1 (MAdCAM-1), triggering receptors expressed on myeloid cells-1 (Trem-1), Colony-stimulating factor 1 receptor (CSF-1R), OX40, 4-1BB, TNF-alpha receptor 1 (TNFR-1), TNF-alpha receptor 1 (TNFR-2), a receptor for B lymphocyte stimulator (BLyS), and an Interferon-alpha receptor.

2. Other Domains of Carrier Moieties

The carrier moieties may also comprise other domains that are not antigen-binding. For example, an antibody Fc domain (e.g., a human IgG₁, IgG₂, IgG₃, or IgG₄ Fc), a polymer (e.g., PEG), an albumin (e.g., a human albumin) or a fragment thereof, or a nanoparticle can be used.

By way of example, the cytokine agonist polypeptide (e.g., IL-10 or TGF-β) and its antagonist may be fused to an antibody Fc domain, forming an Fc fusion protein. In some embodiments, the cytokine agonist polypeptide is fused (directly or through a peptide linker) to the C-terminus or N-terminus of one of the Fc domain polypeptide chains, and the cytokine mask is fused to the corresponding C-terminus or N-terminus of the other Fc domain polypeptide chain through a cleavable peptide linker, wherein the two Fc domain polypeptide chains contain mutations that allow the specific pairing of the two different Fc chains. In some embodiments, the Fc domain comprises the knobs-into-holes mutations described above. In further embodiments, the Fc domain also comprise the YTE and/or LALA mutations described above.

The carrier moiety of the prodrug may comprise an albumin (e.g., human serum albumin) or a fragment thereof. In some embodiments, the carrier moiety comprises an albumin fragment (e.g., a human serum albumin fragment) that is about 10 or more, 20 or more, 30 or more 40 or more, 50 or more, 60 or more, 70 or more, 80 or more, 90 or more, 100 or more, 120 or more, 140 or more, 160 or more, 180 or more, 200 or more, 250 or more, 300 or more, 350 or more, 400 or more, 450 or more, 500 or more, or 550 or more amino acids in length. In some embodiments, the albumin fragment is between about 10 amino acids and about 584 amino acids in length (such as between about 10 and about 20, about 20 and about 40, about 40 and about 80, about 80 and about 160, about 160 and about 250, about 250 and about 350, about 350 and about 450, or about 450 and about 550 amino acids in length). In some embodiments, the albumin fragment includes the Sudlow I domain or a fragment thereof, or the Sudlow II domain or the fragment thereof.

In some embodiments, the carrier is an antibody Fc fragment. Fc is a dimeric molecule that has two N-terminals and two C-terminals. In some embodiments, the cytokine moiety can be fused to one Fc polypeptide in a dimeric Fc fragment, and the masking moieties can be fused to the 2^{nd} Fc polypeptide. In a preferred embodiment, both the cytokine moiety and the masking moiety are fused to the C-terminal of each polypeptide chain of the dimeric Fc fragment. In some embodiments, both the cytokine moiety and the masking moieties are fused to the N-terminal of each polypeptide chain of the dimeric Fc fragment.

D. Linker Components of the Prodrugs

The moieties of the prodrugs disclosed herein may be fused to each other through one or more peptides linkers (e.g., non-cleavable linker or cleavable linker). The cytokine moiety may be fused to the carrier moiety with or without a peptide linker. The peptide linker may be non-cleavable and may be selected from GGGGS (SEQ ID NO: 117), GGGGSGGGGS (SEQ ID NO: 118), GGGGSGGGGSGGGGS (SEQ ID NO: 119), and GGGGSGGGGSAAGGGGSGGGGS (SEQ ID NO: 120).

The cleavable linker may contain one or more (e.g., two or three) cleavable moieties (CM). Each CM may be a substrate for an enzyme or protease selected from legumain, plasmin, TMPRSS-3/4, MMP2, MMP3, MMP9, MT1-MMP, cathepsin, caspase, human neutrophil elastase, beta-secretase, uPA, EOS, and PSA. Examples of cleavable linkers include, without limitation, those comprising an amino acid sequence selected from SEQ ID NOs: 75-95. The peptide linker may be a cleavable peptide linker comprising PYAYWMR (SEQ ID NO: 76). In some embodiments, the cleavable linker is no more than 10 amino acids, or no more than 8 amino acids, or no more than 6 amino acids in length.

### II. Pharmaceutical Compositions

Pharmaceutical compositions of the prodrugs or chimeric molecules are prepared by mixing the presently disclosed prodrugs or chimeric molecules having the desired degree of purity with one or more optional pharmaceutically acceptable carriers (see Osol, A. Ed. Remington's Pharmaceutical Sciences 16th edition (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof, such as citrate, phosphate, succinate, tartrate, fumarate, gluconate, oxalate, lactate, acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl, or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter- and intra-molecular interactions. Tonicity agents can be present in any amount between 0.1% to 25% by weight, or more preferably between 1% to 5% by weight, taking into account the relative amounts of the other ingredients. Preferred tonicity agents include polyhydric sugar alcohols, preferably trihydric or higher sugar alcohols, such as glycerin, erythritol, arabitol, xylitol, sorbitol, and mannitol.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

The choice of pharmaceutical carrier, excipient or diluent may be selected with regard to the intended route of administration and standard pharmaceutical practice. Pharmaceutical compositions may comprise as—or in addition to—the carrier, excipient, or diluent any suitable binder(s), lubricant(s), suspending agent(s), coating agent(s) or solubilizing agent(s).

There may be different composition/formulation requirements dependent on the different delivery systems. By way of example, pharmaceutical compositions useful in the present invention may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular, or subcutaneous route. Alternatively, the formulation may be designed to be administered by a number of routes. In some embodiments, said formulation is administrated topically.

In some embodiments, an antibody or protein formulation is a lyophilized formulation. In some embodiments, an antibody or protein formulation is an aqueous formulation.

In some embodiments, the pharmaceutical composition is a combination pharmaceutical composition, which comprises a chimeric molecule or prodrug of the present invention, a pharmaceutically acceptable excipient, and a second active ingredient selected from a different cytokine or its fusion molecule, an antibody against TNFα, an antibody against IL-6, an antibody against IL-17, an antibody against IL-23, and an IL-2 mutein which selectively stimulate and activate Treg cells.

### III. Methods of Treatment

The presently disclosed chimeric molecules or prodrugs can be used to treat a disease, depending on the antigen bound by the antigen-binding domain. In some embodiments, the chimeric molecule or prodrugs disclosed herein are used to treat a disease such as an autoimmune disease or an inflammation disease.

In some embodiments, a method of treating a disease in a subject comprises administering to the subject an effective amount of the presently disclosed chimeric molecule or prodrugs.

In some embodiments, the autoimmune disease or inflammation disease or condition is selected from the group consisting of rheumatoid arthritis, multiple sclerosis, osteoarthritis, psoriasis, graft vs host disease (GvHD), lupus (e.g., SLE), a neurodegeneration disease (e.g., Alzheimer's disease), an inflammatory bowel disease, ulcerative colitis, Crohn's disease, NASH, atherosclerosis, and systemic sclerosis.

Generally, dosages and routes of administration of the present pharmaceutical compositions are determined according to the size and condition of the subject, according to standard pharmaceutical practice. In some embodiments, the pharmaceutical composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, topically, by nebulization, intrapleurally, intraventricularly, intra-articularly, intraocularly, or intraspinally. In some embodiments, the composition is administered to a subject intravenously.

In some embodiments, the prodrug is administered to a subject in need a single dose or a repeated dose. In some embodiments, the doses are given to a subject once per day, twice per day, three times per day, or four or more times per day. In some embodiments, about 1 or more (such as about 2, 3, 4, 5, 6, or 7 or more) doses are given in a week. In some embodiments, the antibody fusion molecule conjugated to the drug is administered weekly, once every 2 weeks, once every 3 weeks, once every 4 weeks, weekly for two weeks out of 3 weeks, or weekly for 3 weeks out of 4 weeks. In some embodiments, multiple doses are given over the course of days, weeks, months, or years. In some embodiments, a course of treatment is about 1 or more doses (such as about 2, 2, 3, 4, 5, 7, 10, 15, or 20 or more doses).

In some embodiments, the chimeric molecule or prodrug is administered to a subject in combination with a second pharmaceutical composition, wherein the second pharmaceutical composition comprises an active ingredient selected from another cytokine or its fusion molecule such as an IL-2 mutein which selectively stimulate Treg cells, an antibody against TNFα, an antibody against IL-1, an antibody against IFN-γ, an antibody against IFNα, an antibody against IL-6, an antibody against IL-17, and an antibody against IL-23.

### IV. Methods of Making the Prodrugs

The presently disclosed prodrugs or chimeric molecules can be produced using recombinant DNA methods. Nucleic acid molecules encoding the polypeptide or the fusion polypeptide of the prodrug can be isolated and inserted into one or more vectors for further cloning and/or expression in a host cell. Such nucleic acid molecules may be readily isolated and sequenced using conventional methods. Suitable host cells for cloning or expression of fusion polypeptide vectors include prokaryotic cells or eukaryotic cells. Exemplary host cells include Chinese Hamster Ovary (CHO) cells or human embryonic kidney cells (e.g., HEK293).

Expression host cells express the antibody fusion molecule. After an expression period, the host cells can be lysed and the prodrug or antibody fusion molecule can be purified. Exemplary purification methods include liquid chromatography, such as ion exchange chromatography, affinity chromatography (such as Protein A affinity chromatography), or size exclusion chromatography.

It is understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is not intended to be limited to a particular compound, composition, article, or method, unless expressly stated as such. In addition, those of ordinary skill in the art will recognize that certain changes, modification, permutations, alterations, additions, subtractions, and sub-combinations thereof can be made in accordance with the teachings herein without departing from the spirit of the present specification.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It is understood that aspects and variations of the invention described herein include “consisting” and/or “consisting essentially of” aspects and variations. All publications and other references mentioned herein are incorporated by reference in their entirety. Although a number of documents are cited herein, this citation does not constitute an admission that any of these documents forms part of the common general knowledge in the art.

### V. Exemplary Embodiments

In order that this invention may be better understood, the following exemplary embodiments are set forth. These embodiments are for purposes of illustration only and are not to be construed as limiting the scope of the invention in any manner.

1. A chimeric molecule which modulates a function of an immune cell and is useful to treat a patient with an autoimmune disease or an inflammatory condition, comprising:
a. a carrier which comprises an antigen-binding moiety which binds to an antigen expressed on the surface of an immune cell;
b. a cytokine moiety which comprises a cytokine selected from a TGF-β1 agonist polypeptide, a TGF-β2 agonist polypeptide, a TGF-β3 agonist polypeptide, and an IL-10 agonist polypeptide; and wherein said immune cell also express a receptor for TGF-β or IL-10.
   2. The chimeric molecule of embodiment 1, which further comprises a masking moiety; wherein said masking moiety binds to said cytokine moiety and inhibits a biological activity of said cytokine moiety.
   3. The chimeric molecule of embodiment 1 or 2, wherein said antigen-binding moiety binds to one or more antigens selected from IL-1 receptor accessory protein (IL1RAP), IL-1 receptor (IL-1RI), a human IL-3 receptor, IL-4 receptor α chain (IL-4Rα), IL-5 receptor α chain (IL-5Rα), IL-6 receptor α chain (IL-6Rα), a human IL-9 receptor, a human IL-13 receptor, a human IL-17 receptor, a human IL-23 receptor, a human IL-31 receptor, a human IL-33 receptor, a receptor for thymic stromal lymphopoietin (TSLP), CD20, CD25, BCMA, CD40, CD80, CD86, Trem-1, CSF-1R, OX40, 4-1BB, TNF-alpha receptor 1 (TNFR-1), TNF-alpha receptor 1 (TNFR-2), a receptor for B lymphocyte stimulator (BLyS), and an Interferon-alpha receptor.
   4. The chimeric molecule of any one of embodiments 1-3, wherein said cytokine moiety comprises a human IL-10 agonist polypeptide with an amino acid sequence of SEQ ID NO: 1, 2, or 3 or at least 90% identical as SEQ ID NO: 1, 2, or 3; and wherein said masking moiety comprises an extracellular domain of IL-10 receptor α chain (IL-10Rα-ECD), an analog of IL-10Rα-ECD, or an antibody against human IL-10 or a binding fragment thereof.
   5. The chimeric molecule of any one of embodiments 1-3, wherein said cytokine moiety comprises a human IL-10 agonist polypeptide with an amino acid sequence of SEQ ID NO: 1, 2, or 3 or at least 90% identical as SEQ ID NO: 1, 2, or 3; and wherein said masking moiety comprises an amino acid sequence of SEQ ID NO: 4, 5, or 6, or at least 95% identical as SEQ ID NO: 4, 5, or 6.
   6. The chimeric molecule of any one of embodiments 1-3, wherein said cytokine moiety comprises a human TGF-β agonist polypeptide with an amino acid sequence of SEQ ID NO: 7 or at least 90% identical as SEQ ID NO: 7; and wherein said masking moiety comprises an extracellular domain of TGF-β Receptor II (TGFRII-ECD), an analog of TGFRII-ECD, or an antibody against human TGF-β or a binding fragment thereof.
   7. The chimeric molecule of any one of embodiments 1-3, wherein said cytokine moiety comprises a human TGF-β agonist polypeptide with an amino acid sequence of SEQ ID NO: 7 or at least 90% identical as SEQ ID NO: 7; and wherein said masking moiety comprises an amino acid sequence of SEQ ID NO: 10 or at least 95% identical as that of SEQ ID NO: 10.
   8. The chimeric molecule of embodiment 6, wherein said masking moiety is a scFv which binds to human TGF-β; wherein said scFv comprises a VH domain with an amino acid sequence of SEQ ID NO: 9 or at least 95% identical as SEQ ID NO:9, and a VL domain with an amino acid sequence of SEQ ID NO: 8 or at least 95% identical as SEQ ID NO:8.
   9. The chimeric molecule of any one of embodiments 1-8, wherein said carrier moiety comprises an anti-IL-4 receptor α chain (IL-4Rα) antibody or a binding fragment thereof, which comprises light chain CDRs as derived from SEQ ID NO: 11, and heavy chain CDRs as derived from SEQ ID NO: 12.
   10. The chimeric molecule of any one of embodiments 1-8, wherein said carrier moiety comprises an anti-IL-4 receptor α chain (IL-4Rα) antibody or a binding fragment thereof, which comprises a light chain variable domain with an amino acid sequence of SEQ ID NO: 13 or at least 95% identical as that of SEQ ID NO: 13, and a heavy chain variable domain with an amino acid sequence of SEQ ID NO: 14 or at least 95% identical as that of SEQ ID NO: 14.
   11. The chimeric molecule of any of embodiments 1-8, wherein said carrier moiety comprises an anti-IL-5 receptor α chain (IL-5Rα) antibody (benralizumab) or a fragment thereof, which comprises a light chain variable domain with an amino acid sequence of SEQ ID NO: 15 or at least 95% identical as that of SEQ ID NO: 15, and a heavy chain variable domain with an amino acid sequence of SEQ ID NO: 16 or at least 95% identical as that of SEQ ID NO: 16.
   12. The chimeric molecule of any one of embodiments 1-8, wherein said carrier moiety comprises an anti-IL-6 receptor α chain (IL-6Rα) antibody or a fragment thereof, which comprises a light chain variable domain with an amino acid sequence of SEQ ID NO: 17 or at least 95% identical as that of SEQ ID NO: 17, and a heavy chain variable domain with an amino acid sequence of SEQ ID NO: 18 or at least 95% identical as that of SEQ ID NO: 18.
   13. The chimeric molecule of any one of embodiments 1-8, wherein said carrier moiety comprises an anti-IL-6 receptor α chain (IL-6Rα) antibody or a fragment thereof, which comprises a light chain variable domain with an amino acid sequence of SEQ ID NO: 19 or at least 95% identical as that of SEQ ID NO: 20, and a heavy chain variable domain with an amino acid sequence of SEQ ID NO: 19 or at least 95% identical as that of SEQ ID NO: 20.
   14. The chimeric molecule of any one of embodiments 1-8, wherein said carrier moiety comprises an anti-Trem-1 antibody or a fragment thereof, which comprises a light chain variable domain with an amino acid sequence of SEQ ID NO: 21 or at least 95% identical as that of SEQ ID NO: 21, and a heavy chain variable domain with an amino acid sequence of SEQ ID NO: 22 or at least 95% identical as that of SEQ ID NO: 22.
   15. The chimeric molecule of any one of embodiments 1-8, wherein said carrier moiety comprises an anti-CD86 antibody or a fragment thereof, which comprises a light chain variable domain with an amino acid sequence of SEQ ID NO: 23 or at least 95% identical as that of SEQ ID NO: 23, and a heavy chain variable domain with an amino acid sequence of SEQ ID NO: 24 or at least 95% identical as that of SEQ ID NO: 24.
   16. The chimeric molecule of any one of embodiments 1-8, wherein said carrier moiety comprises an extracellular domain of CTLA-4 (CTLA-4-ECD) or a fragment or analog thereof, which comprises an amino acid sequence of SEQ ID NO: 25 or 61 or at least 95% identical as that of SEQ ID NO: 25 or 61.
   17. The chimeric molecule of any one of embodiments 1-8, wherein said carrier moiety comprises an anti-interferon alpha receptor 1 (IFNRA-1) antibody or a fragment thereof, which comprises a light chain variable domain with an amino acid sequence of SEQ ID NO: 52 or at least 95% identical as that of SEQ ID NO: 52, and a heavy chain variable domain with an amino acid sequence of SEQ ID NO: 51 or at least 95% identical as that of SEQ ID NO: 51.
   18. The chimeric molecule of any one of embodiments 1-8, wherein said carrier moiety comprises an anti-CD86 antibody or a fragment thereof, which comprises a light chain variable domain with an amino acid sequence of SEQ ID NO: 23 or at least 95% identical as that of SEQ ID NO: 23, and a heavy chain variable domain with an amino acid sequence of SEQ ID NO: 24 or at least 95% identical as that of SEQ ID NO: 24.
   19. The chimeric molecule of any one of embodiments 1-16, which comprises one or more peptide linkers; wherein said peptide linkers are not cleavable, which are optionally selected from SEQ ID NOs: 95-99.
   20. A chimeric molecule which comprises two identical light chains and a first heavy chain polypeptide chain and a second heavy chain polypeptide chain; wherein said light chain comprises an amino acid sequence of SEQ ID NO: 26 or at least 95% identical as that of SEQ ID NO: 26; said first heavy chain polypeptide chain comprises an amino acid sequence of SEQ ID NO: 27 or 28, or at least 95% identical as that of SEQ ID NO: 27 or 28; and said second heavy chain polypeptide chain comprises an amino acid sequence of SEQ ID NO: 29 or 30 or at least 95% identical as that of SEQ ID NO: 29 or 30.
   21. A chimeric molecule which comprises two identical light chains and two identical heavy chain polypeptide chains; wherein said light chain comprises an amino acid sequence of SEQ ID NO: 26 or at least 95% identical as that of SEQ ID NO: 26; said heavy chain polypeptide chain comprises an amino acid sequence of SEQ ID NO: 31, 32, 33, 34, or 100 or at least 95% identical as that of SEQ ID NO: 31, 32, 33, 34, or 100.
   22. A chimeric molecule which comprises two identical light chains and a first heavy chain polypeptide chain and a second heavy chain polypeptide chain; wherein said light chain comprises an amino acid sequence of SEQ ID NO: 35 or at least 95% identical as that of SEQ ID NO: 35; said first heavy chain polypeptide chain comprises an amino acid sequence of SEQ ID NO: 36 or at least 95% identical as that of SEQ ID NO: 36; and said second heavy chain polypeptide chain comprises an amino acid sequence of SEQ ID NO: 37 or 38 or at least 95% identical as that of SEQ ID NO: 37 or 38.
   23. A chimeric molecule which comprises two identical light chains and two identical heavy chain polypeptide chains; wherein said light chain comprises an amino acid sequence of SEQ ID NO: 35 or at least 95% identical as that of SEQ ID NO: 35; said heavy chain polypeptide chain comprises an amino acid sequence of SEQ ID NO: 39, 40, 41 or 42 or at least 95% identical as that of SEQ ID NO: 39, 40, 41 or 42.
   24. A chimeric molecule which comprises two identical light chains and a first heavy chain polypeptide chain and a second heavy chain polypeptide chain; wherein said light chain comprises an amino acid sequence of SEQ ID NO: 43 or at least 95% identical as that of SEQ ID NO: 43; said first heavy chain polypeptide chain comprises an amino acid sequence of SEQ ID NO: 44 or at least 95% identical as that of SEQ ID NO: 44; and said second heavy chain polypeptide chain comprises an amino acid sequence of SEQ ID NO: 45 or 46 or at least 95% identical as that of SEQ ID NO: 45 or 46.
   25. A chimeric molecule which comprises two identical light chains and two identical heavy chain polypeptide chains; wherein said light chain comprises an amino acid sequence of SEQ ID NO: 43 or at least 95% identical as that of SEQ ID NO: 43; said heavy chain polypeptide chain comprises an amino acid sequence of SEQ ID NO: 47, 48, 49 or 50 or at least 95% identical as that of SEQ ID NO: 47, 48, 49, or 50.
   26. A chimeric molecule which comprises two identical light chains and a first heavy chain polypeptide chain and a second heavy chain polypeptide chain; wherein said light chain comprises an amino acid sequence of SEQ ID NO: 53 or at least 95% identical as that of SEQ ID NO: 53; said first heavy chain polypeptide chain comprises an amino acid sequence of SEQ ID NO: 54 or at least 95% identical as that of SEQ ID NO: 54; and said second heavy chain polypeptide chain comprises an amino acid sequence of SEQ ID NO: 55 or 56 or at least 95% identical as that of SEQ ID NO: 55 or 56.
   27. A chimeric molecule which comprises two identical light chains and two identical heavy chain polypeptide chains; wherein said light chain comprises an amino acid sequence of SEQ ID NO: 53 or at least 95% identical as that of SEQ ID NO: 53; said heavy chain polypeptide chain comprises an amino acid sequence of SEQ ID NO: 57, 58, 59 or 60 or at least 95% identical as that of SEQ ID NO: 57, 58, 59, or 60.
   28. A chimeric molecule which comprises two identical polypeptide chains, which comprises an amino acid sequence of SEQ ID NO: 66, 67, 68, or 69 or at least 95% identical as that of SEQ ID NO: 66, 67, 68, or 69.
   29. A chimeric molecule which comprises 1 first polypeptide chain and a second polypeptide chain which form a heterodimer; wherein said first polypeptide chain comprises an amino acid sequence of SEQ ID NO: 63 or at least 95% identical as that of SEQ ID NO: 63, and said second polypeptide chain comprises an amino acid sequence of SEQ ID NO: 64 or 65 or at least 95% identical as that of SEQ ID NO: 64 or 65.
   30. The chimeric molecule of any one of embodiments 1-16, which comprises a cleavable peptide linker, which is cleavable by a protease preferentially expressed at an inflammation site.
   31. The chimeric molecule of any one of embodiments 1-16, which comprises a cleavable peptide linker, which comprises an amino acid sequence selected from SEQ ID NOs: 75-94.
   32. The chimeric molecule of any one of embodiments 1-31, which has higher activity modulating an immune cell which expresses both the antigen targeted by the carrier moiety and a receptor for IL-10 or TGF-β than an immune cell which does not express said antigen and/or said cytokine receptor.
   33. A chimeric molecule which modulates a function of an immune cell and is useful to treat a patient with an autoimmune disease or an inflammatory condition, comprising:
a. a carrier which comprises a neutralizing antibody or a binding fragment thereof which binds to and neutralize a biological activity of a cytokine selected from IL-1α, IL-1β, IL-4, IL-5, IL-6, 11-12, IL-13, IL-17, IL-23, IL-31, IL-33, tumor necrosis factor alpha (TNFα), and interferon alpha (IFNα), interferon gamma (IFNγ) or binds to a chemokine;
b. a cytokine moiety which comprises a cytokine selected from a TGF-β1 agonist polypeptide, a TGF-β2 agonist polypeptide, a TGF-β3 agonist polypeptide, and an IL-10 agonist polypeptide;
c. a masking moiety which binds to the IL-10 agonist polypeptide or the TGF-β agonist; and
d. a cleavable peptide linker which links said masking moiety to the carrier moiety or the cytokine moiety; or a cleavable peptide linker which links said cytokine moiety to the carrier moiety or the masking moiety.
   34. The chimeric molecule of embodiment 33, wherein said neutralizing antibody comprises the same light chain variable domain and the same heavy chain variable domain as an antibody selected from canakinumab, adalimumab, CDP-571, infliximab, rontalizumab, sifalimumab, olokizumab (CDP6038), elsilimomab, BMS-945429 (ALD518), sirukumab (CNTO 136), levilimab (BCD-089), siltuximab, secukinumab, ixekizumab, ustekinuma, guselkumab and tildrakizumab.
   35. The chimeric molecule of embodiment 33, wherein said neutralizing antibody comprises the same light chain CDR domains and the same heavy chain domains as derived from an antibody selected from canakinumab, adalimumab, CDP-571, infliximab, rontalizumab, sifalimumab, olokizumab (CDP6038), elsilimomab, BMS-945429 (ALD518), sirukumab (CNTO 136), levilimab (BCD-089), siltuximab, secukinumab, ixekizumab, ustekinuma, guselkumab and tildrakizumab.
   36. The chimeric molecule of any one of the embodiments 33-35, wherein said cytokine moiety comprises one or two copies of an IL-10 agonist polypeptide, which comprises an amino acid sequence of SEQ ID NO: 1, 2, or 3, or at least 95% identical as that of SEQ ID NO: 1, 2, or 3.
   37. The chimeric molecule of any one of the embodiments 33-35, wherein said cytokine moiety comprises one or two copies of a TGF-β agonist polypeptide, which comprises an amino acid sequence of SEQ ID NO: 7, or at least 95% identical as that of SEQ ID NO: 7; and wherein said masking moiety comprises an amino acid sequence of SEQ ID NO: 10 or at least 95% identical as that of SEQ ID NO: 10.
   38. The chimeric molecule of any one of the embodiments 33-36, wherein said masking moiety comprises one or two copies of an IL-10 antagonist, which comprises an amino acid sequence selected from SEQ ID NO: 4, 5, or 6, or at least 95% identical as that of SEQ ID NO: 4, 5, or 6.
   39. The chimeric molecule of any one of embodiments 33-38, wherein said cleavable peptide linker is cleavable by a protease preferentially expressed at a site of inflammation.
   40. The chimeric molecule of any one of embodiments 33-38, wherein said cleavable peptide linker comprises an amino acid sequence selected from SEQ ID NOs: 75-94.
   41. A chimeric molecule which comprises two identical polypeptide chains, which comprises an amino acid sequence of SEQ ID NO: 73 or 74 or at least 95% identical as that of SEQ ID NO: 73 or 74.
   42. A chimeric molecule which comprises a first polypeptide chain and a second polypeptide chain which form a heterodimer; wherein said first polypeptide chain comprises an amino acid sequence of SEQ ID NO: 70 or at least 95% identical as that of SEQ ID NO: 70, and said second polypeptide chain comprises an amino acid sequence of SEQ ID NO: 71 or 72 or at least 95% identical as that of SEQ ID NO: 71 or 72.
   43. A pharmaceutical composition comprising the chimeric molecule of any one of embodiments 1-42 as its active ingredient, and a pharmaceutically acceptable excipient.
   44. A polynucleotide or polynucleotides encoding the chimeric molecule of any one of embodiments 1-42.
   45. An expression vector or vectors comprising the polynucleotide or polynucleotides of embodiment 44.
   46. A host cell comprising the vector(s) of embodiment 45.
   47. A method of making the chimeric molecule of any one of embodiments 1-42, comprising culturing the host cell of embodiment 46 under conditions that allow expression of the chimeric molecule, and isolating the chimeric molecule.
   48. A method of treating an autoimmune disease or inflammation in a patient in need thereof, comprising administering the patient with the pharmaceutical composition of embodiment 43.
   49. A method of treating a patient with cancer, an autoimmune disease or an inflammation condition, comprising administering the patient with pharmaceutical composition of embodiment
   43, wherein said patient is also administrated with a pharmaceutical composition which comprises an IL-2 mutein, an antagonist of TNFα, an antagonist of IL-12, an antagonist of IL-17 or its receptor, an antagonist of IL-23 or its receptor, an antagonist of IL-6 or its receptor, an antagonist of IL-5 or its receptor, an antagonist of IL-4 or its receptor, an antagonist of IL-1β or its receptor, an antagonist of interferon alpha receptor-1 (INFAR-1), an antagonist of CD40, an antagonist of CD80, or an antagonist of CD86.
   50. The method of embodiment 48, or 49, wherein said autoimmune disease or inflammation condition is selected from the group consisting of asthma, atopic dermatitis, Type I diabetes, diabetic ulcers, allergy, psoriasis, rheumatoid arthritis, osteoarthritis, graft vs host disease (GvHD), lupus nephritis, systemic lupus erythematosus (SLE), Alzheimer's disease, a neuron degeneration disease, an inflammatory bowel disease, ulcerative colitis, Crohn's disease NASH, atherosclerosis, and systemic sclerosis.

### EXAMPLES

### Example 1: Transient Transfection of ExpiCHO™ Cells

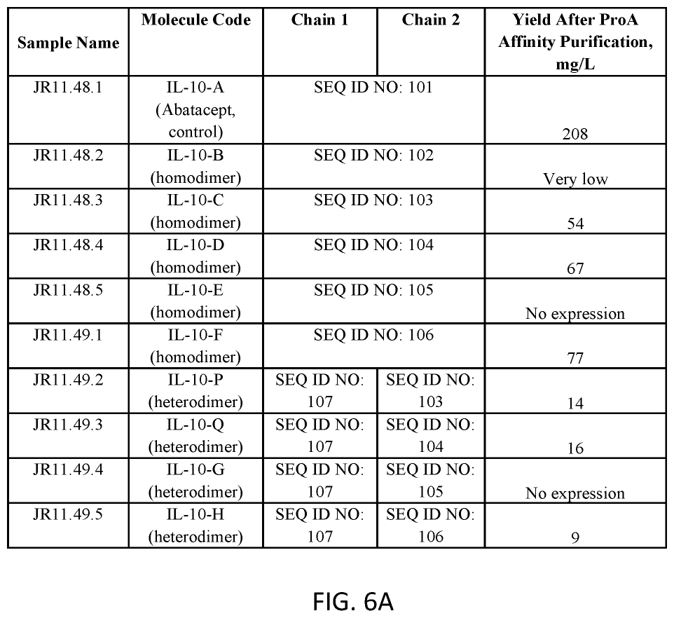

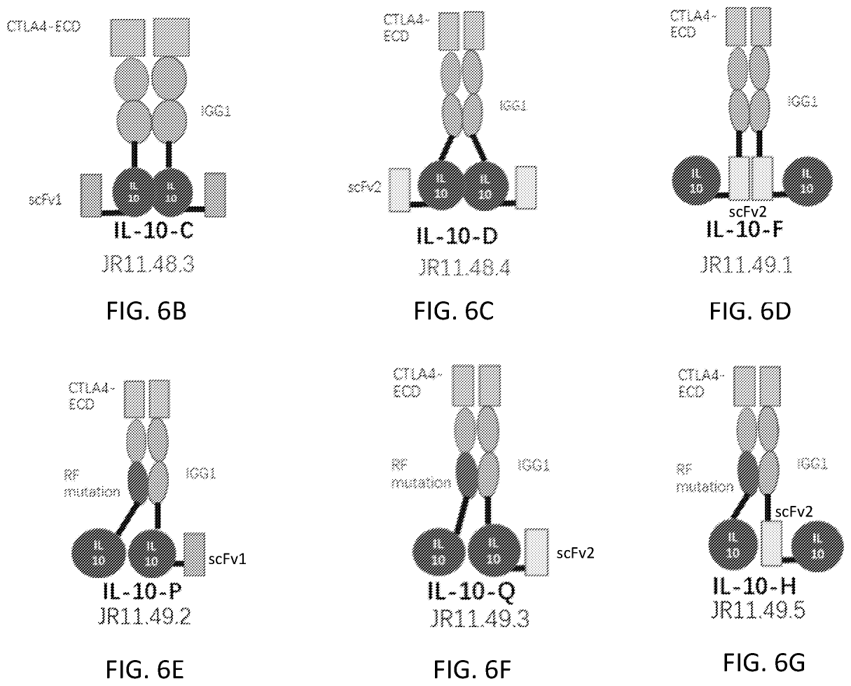

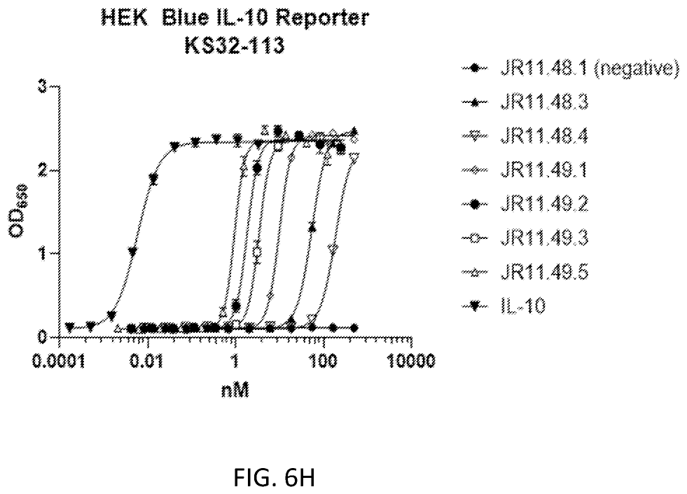

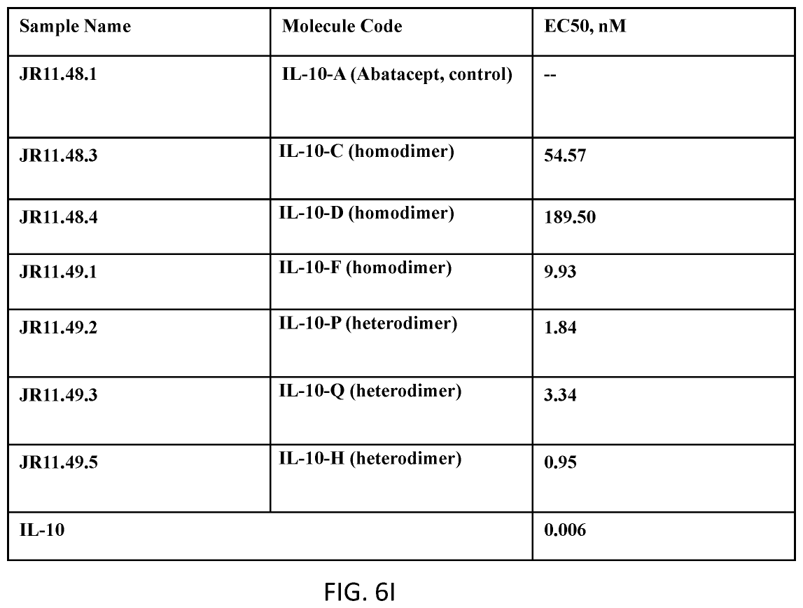

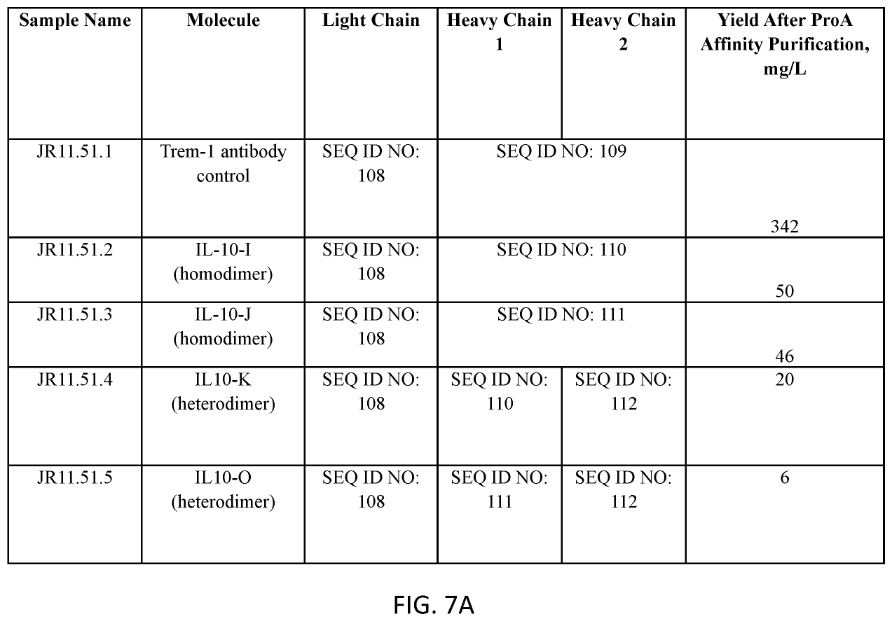

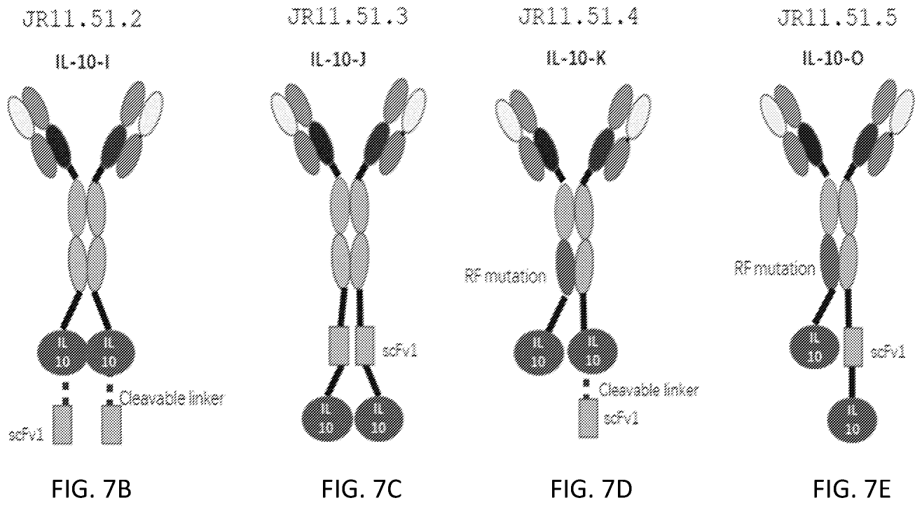

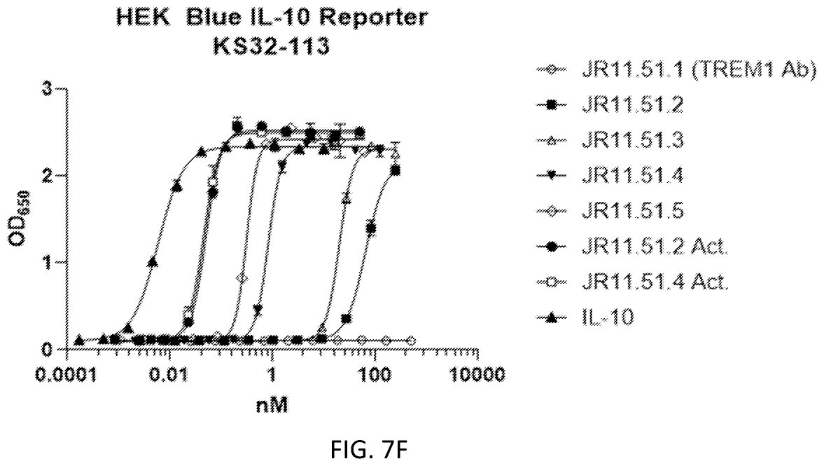

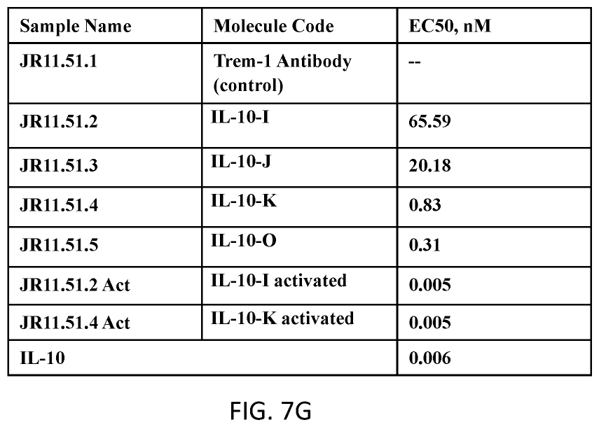

Expression plasmids were co-transfected into 6×10 6 cell/ml freestyle ExpiCHO™ cells at 1 μg/ml using ExpiFectamine™ CHO Transfection kit (Gibco). For the IL-10 prodrug molecules of two or more polypeptide chains, the ratios of the various chains were tested to achieve good transient expression titers. FIG. 6A shows the sequence IDs of the CTLA-4 ECD-Fc-based IL-10 prodrugs. FIG. 7A shows the sequence IDs of the Trem-1 antibody-IL-10 prodrug molecules. The cell cultures were harvested 7 days after transfection by centrifuging at 9,000 rpm for 45 min followed by 0.22 μM filtration.

### Example 2: Purification of IL-10 Prodrugs

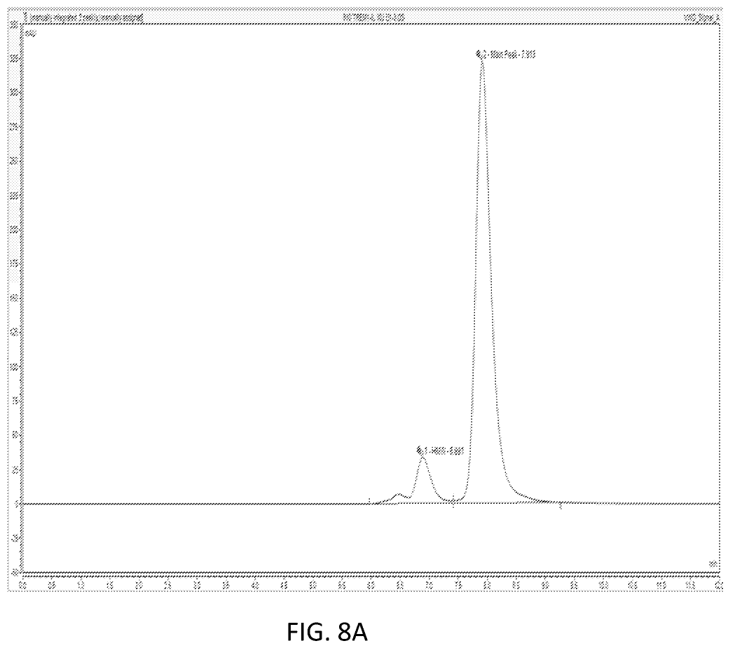

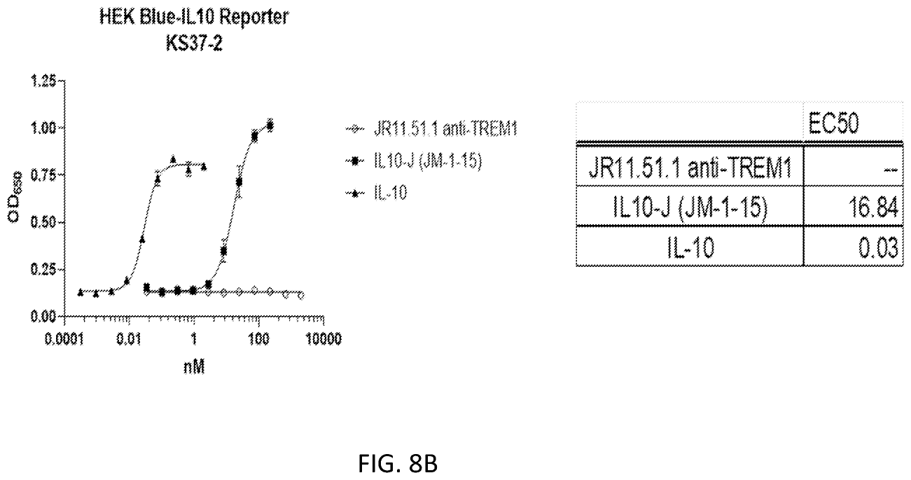

The purifications of the proteins of the IL-10 prodrugs were carried out using Protein A affinity chromatography. Additional purification steps were carried out using additional chromatography and filtration steps. For example, chromatography steps with resins such as Capto™ MMC ImpRes, Capto™ Adhere, Capto™ SP, and/or Q Sepharose FF can be used for further purification of the prodrugs. For the prodrug IL-10-J, it was further purified by Capto™ MMC Impres after the Protein A Affinity Chromatography. Its purity was analyzed by SEC-HPLC (FIG. 8A) and activity by the HEK-Blue™ IL-10 Reporter Assay (FIG. 8B).

### Example 3: SEC-HPLC Analysis

SEC-HPLC was carried out using an Agilent 1100 Series of HPLC system with a TSKgel® G3000SWXL column (7.8 mmIDX 30 cm, 5 μm particle size) ordered from Tosoh Bioscience. A sample of up to 10011.1 was loaded. The column was run with a buffer containing 200 mM K3PO₄, 250 mM KCl, pH 6.5. The flow rate was 0.5 ml/min. The column was run at room temperature. The protein elution was monitored both at 220 nm and 280 nm. FIG. 8A shows the SEC-HPLC chromatograph of the prodrug IL-10-J (sample name: JM-1-15) after purification.

### Example 4: Proteolytic Treatment

One μg of the protease, human MMP-2 (R&D systems), human MMP-9 (R&D systems), mouse MMP-2 (R&D systems), or mouse MMP-9 (R&D systems) was added to 50 of the precursor protein, and incubated at 37° C. overnight.

### Example 5: HEK-Blue™ IL-10 Reporter Assay

Interleukin-10 binding to its receptor causes oligomerization of the IL-10Rα chain and IL-10Rβ chain. This allows phosphorylation of JAK1 and TyK2 resulting in activation of STAT3. Functional activity of IL-10 was measured using a secreted alkaline phosphatase reporter cell line (HEK-Blue™ IL-10, InvivoGen). This reporter cell line consists of HEK293 cells stably transfected with the IL10Rα and IL10Rβ chains, human STAT3 and a secreted alkaline phosphatase (SEAP) reporter. The reporter gene is under control of the IFN-β minimal promoter fused to AP1 and STAT3 binding sites. The cell line is also knockout for the expression of human IFNAR2 and human IL-6R to prevent STAT3 signaling from other cytokines known to activate STAT3. Culturing HEK-Blue™ IL-10 cells with IL-10 activates JAK1/STAT3 resulting in the production of SEAP in the culture supernatant, which is quantitated using Quanti-Blue™ Solution (InvivoGen). Test articles were serially diluted in 50 μL/well in 96-well tissue culture plates. HEK-Blue™ IL-10 cells were added at 30,000 cells/well/50 μL. Cultures were incubated overnight at 37° C., and 20 μL of culture supernatant was transferred to an ELISA plate and 180 μL of Quanti-Blue™ Solution is added. Plates were incubated at 37° C. for 1 hour, and absorbance was measured with a spectrophotometer at 450 nm.

The prodrugs of FIGS. 6E-6G comprise RF mutations (H371R/Y372F; numbering according to SEQ ID NO: 107) in the Fc domain to reduce or eliminate the binding of the Fc domain to a Protein A affinity resin. FIG. 6H shows the reporter assay results of the IL-10 prodrug molecules with CTLA-4 ECD-Fc as the carrier. The activities of the negative control molecule (CTLA-4 ECD-Fc) and the positive control (IL-10) were also included in the assay. As shown in FIG. 6I, all the prodrugs had significantly lower activities compared to IL-10. In addition, the homodimer IL-10 prodrug molecule samples JR11.48.3, JR11.48.4, and JR11.49.1 have two masking moieties and had significantly lower activities compared to the ones with one masking moiety (JR11.49.2, JR11.49.3, and JR11.49.5).

FIG. 7C shows the reporter assay results of the IL-10 prodrug molecules with the Trem-1 antibody as the carrier. The activities of the carrier control and the positive control (IL-10) were also included in the assay. In addition, IL-10-I (FIG. 7B) and IL-10-K (FIG. 7D) after activation were also tested. As shown in FIG. 7G, all the prodrugs had significantly lower activities compared to IL-10. In addition, the IL-10 prodrug molecule samples with two masking moieties had significantly lower activities compared to the ones with one masking moiety. Further, both IL-10-I (FIG. 7B) and IL-10-K (FIG. 7D) had significantly higher activity after activation compared to the ones prior to the activation (FIG. 7G), which demonstrate that the prodrugs are activatable.

FIG. 8B shows the reporter assay activity of the purified IL-10-J sample, which was significantly lower than IL-10. The activity assay results show that both scFv1 and scFv2 worked well as IL-10 masks as both reduced the biological activity of the IL-10 moiety. In addition, the prodrug molecules IL-10-I and IL-10-K masked with scFv1 were both activated after protease cleavage, further demonstrating that scFv1 can serve as the masking moiety for the IL-10 prodrugs.

The above non-limiting examples are provided for illustrative purposes only in order to facilitate a more complete understanding of the disclosed subject matter. These examples should not be construed to limit any of the embodiments described in the present specification, including those pertaining to the antibodies, pharmaceutical compositions, or methods and uses for treating an autoimmune disease or inflammatory condition.

SEQUENCES

SEQ ID NO: 1-human IL-10 (aa 18-178)
SPGQGTQSEN SCTHEPGNLP NMLRDLRDAF SRVKTFFQMK DQLDNLLLKE SLLEDEKGYL
GCQALSEMIQ FYLEEVMPQA ENQDPDIKAH VNSLGENLKT LRLRLRRCHR FLPCENKSKA
VEQVKNAFNK LQEKGIYKAM SEFDIFINYI EAYMTMKIRN
SEQ ID NO: 2-human IL-10 (aa 26-178)
SENSCTHFPG NLPNMLRDLR DAFSRVKTFF QMKDQLDNLL LKESLLEDFK GYLGCQALSE
MIQFYLEEVM PQAENQDPDI KAHVNSLGEN LKTLRLRLRR CHRFLPCENK SKAVEQVKNA
FNKLQEKGIY KAMSEFDIFI NYIEAYMTMK IRN
SEQ ID NO: 3-human IL-10 (aa 18-178, F111S)
SPGQGTQSEN SCTHFPGNLP NMLRDLRDAF SRVKTFFQMK DQLDNLLLKE SLLEDEKGYL
GCQALSEMIQ FYLEEVMPQA ENQDPDIKAH VNSLGENLKT LRLRLRRCHR SLPCENKSKA
VEQVKNAFNK LQEKGIYKAM SEFDIFINYI EAYMTMKIRN
SEQ ID NO: 4-IL-10Ra-ECD
GTELPSPPSV WFEAEFFHHI LHWTPIPNQS ESTCYEVALL RYGIESWNSI SNCSQTLSYD
LTAVTLDLYH SNGYRARVRA VDGSRHSNWT VINTRESVDE VTLTVGSVNL EIHNGFILGK
IQLPRPKMAP ANDTYESIFS HFREYEIAIR KVPGNFTFTH KKVKHENESL LTSGEVGEFC
VQVKPSVASR SNKGMWSKEE CISLTRQYFT VIN
SEQ ID NO: 5-scFvA against IL-10
DIQMTQSPSS LSASVGDRVT ITCKTSQNIF ENLAWYQQKP GKAPKLLIYN ASPLQAGVPS
RFSGSGSGTD FTLTISSLQP EDFATYYCHQ YYSGYTFGPG TKLELKGGGG SGGGGSGGGG
SQVQLVESGG GVVQPGRSLR LSCAASGFTF SDYHMAWVRQ APGKGLEWVA SITLDATYTY
YRDSVRGRFT ISRDNSKNTL YLQMNSLRAE DTAVYYCARH RGESVWLDYW GQGTLVTVSS
SEQ ID NO: 6-scFVB against IL-10
QVQLVESGGG VVQPGRSLRL SCAASGFTES DYHMAWVRQA PGKGLEWVAS ITLDATYTYY
RDSVRGRFTI SRDNSKNTLY LQMNSLRAED TAVYYCARHR GFSVWLDYWG QGTLVTVSSG
GGGSGGGGSG GGGSDIQMTQ SPSSLSASVG DRVTITCKTS QNIFENLAWY QQKPGKAPKL
LIYNASPLQA GVPSRESGSG SGTDFTLTIS SLQPEDFATY YCHQYYSGYT FGPGTKLELK
SEQ ID NO: 7-human TGF-β1
ALDTNYCESS TEKNCCVRQL YIDERKDLGW KWIHEPKGYH ANFCLGPCPY IWSLDTQYSK
VLALYNQHNP GASAAPCCVP QALEPLPIVY YVGRKPKVEQ LSNMIVRSCK CS
SEQ ID NO: 8-VL of an antibody against TGF-β
DIQMTQSPSS LSASVGDRVT ITCRASESVD FYGNSEMHWY QQKPGKAPKL LIYLASNLES
GVPSRFSGSG SGTDETLTIS SLQPEDFATY YCQQNIEDPL TFGGGTKVEI K
SEQ ID NO: 9-VH of an antibody against TGF-β
QVQLVQSGAE VKKPGASVKV SCKASGYTFT SEWMNWVRQA PGQGLEWMGQ IFPALGSTNY
NEMYEGRVTM TTDTSTSTAY MELRSLRSDD TAVYYCARGI GNYALDAMDY WGQGTLVTVS
S
SEQ ID NO: 10-TGF-βRII-ECD
GIPPHVQKSV NNDMIVTDNN GAVKFPQLCK FCDVRESTCD NQKSCMSNCS ITSICEKPQE
VCVAVWRKND ENITLETVCH DPKLPYHDFI LEDAASPKCI MKEKKKPGET FFMCSCSSDE
CNDNIIFSEE YNTSNPD
SEQ ID NO: 11-light chain of an antibody against il-4rβ
MAWMMLLLGL LAYGSGVDSD IVLTQSPASL AVSLGQRATI SCRASLSVSS SGYSHMHWYQ
QKPGQPPKFL IYLASKLQSG VPARESGSGS GTDETLSIHP VEEEDAATYY CQHSRELPFT
FGSGTKLEIK RADAAPTVSI FPPSSEQLTS GGASVVCFLN NFYPKDINVK WKIDGSERQN
GVLNSWTDQD SKDSTYSMSS TLTLTKDEYE RHNSYTCEAT HKTSTSPIVK SENRNEC
SEQ ID NO: 12-heavy chain of an antibody against IL-4Rβ
MAWMMLLLGL LAYGSGVDSD VQLVESGGGL VQPGGSRKLS CAASGFTEST FGMHWVRQTP
EKGLDWVAYI SSGSTTIHYA DSVKGRFTIS RDNPKNTLFL EMTSLRSEDT AMYYCSRRME
RNGMDYWGQG TSVTVSSAKT TAPSVYPLAP VCGDTTGSSV TLGCLVKGYF PEPVTLTWNS
GSLSSGVHTF PAVLQSDLYT LSSSVTVISS TWPSQSITCN VAHPASSTKV DKKIEPRGPT
IKPCPPCKCP APNLLGGPSV FIFPPKIKDV LMISLSPIVT CVVVDVSEDD PDVQISWFVN
NVEVHTAQTQ THREDYNSTL RVVSALPIQH QDWMSGKEFK CKVNNKDLPA PIERTISKPK
GSVRAPQVYV LPPPEEEMTK KQVTLTCMVT DEMPEDIYVE WTNNGKTELN YKNTEPVLDS
DGSYFMYSKL RVEKKNWVER NSYSCSVVHE GLHNHHTTKS FSRTPGK
SEQ ID NO: 13-dupilumab light chain variable domain
DIVMTQSPLS LPVTPGEPAS ISCRSSQSLL YSIGYNYLDW YLQKSGQSPQ LLIYLGSNRA
SGVPDRFSGS GSGTDFTLKI SRVEAEDVGF YYCMQALQTP YTFGQGTKLE IK
SEQ ID NO: 14-dupilumab heavy chain variable domain
EVQLVESGGG LEQPGGSLRL SCAGSGFTER DYAMTWVRQA PGKGLEWVSS ISGSGGNTYY
ADSVKGRETI SRDNSKNTLY LQMNSLRAED TAVYYCAKDR LSITIRPRYY GLDVWGQGTT
VTVSS
SEQ ID NO: 15-IL-5 antibody benralizumab light chain variable domain
DIQMTQSPSS LSASVGDRVT ITCGTSEDII NYLNWYQQKP GKAPKLLIYH TSRLQSGVPS
RFSGSGSGTD FTLTISSLQP EDFATYYCQQ GYTLPYTFGQ GTKVEIK
SEQ ID NO: 16-IL-5 antibody benralizumab heavy chain variable domain
EVQLVQSGAE VKKPGASVKV SCKASGYTFT SYVIHWVRQR PGQGLAWMGY INPYNDGTKY
NERFKGKVTI TSDRSTSTVY MELSSLRSED TAVYLCGREG IRYYGLLGDY WGQGTLVTVS
S
SEQ ID NO: 17-IL-6 antibody tocilizumab Light chain variable domain
DIQMTQSPSS LSASVGDRVT ITCRASQDIS SYLNWYQQKP GKAPKLLIYY TSRLHSGVPS
RFSGSGSGTD FTFTISSLQP EDIATYYCQQ GNTLPYTFGQ GTKVEIK
SEQ ID NO: 18-IL-6 antibody tocilizumab heavy chain variable domain
QVQLQESGPG LVRPSQTLSL TCTVSGYSIT SDHAWSWVRQ PPGRGLEWIG YISYSGITTY
NPSLKSRVTM LRDTSKNQFS LRLSSVTAAD TAVYYCARSL ARTTAMDYWG QGSLVTVSS
SEQ ID NO 19-IL-6 antibody sarilumab light chain variable domain
DIQMTQSPSS VSASVGDRVT ITCRASQGIS SWLAWYQQKP GKAPKLLIYG ASSLESGVPS
RFSGSGSGTD FTLTISSLQP EDFASYYCQQ ANSFPYTFGQ GTKLEIK
SEQ ID NO 20-IL-6 antibody Sarilumab heavy chain variable domain
EVQLVESGGG LVQPGRSLRL SCAASRFTED DYAMHWVRQA PGKGLEWVSG ISWNSGRIGY
ADSVKGRFTI SRDNAENSLF LQMNGLRAED TALYYCAKGR DSFDIWGQGT MVTVSS
SEQ ID NO: 21-VL of antagonist antibody against trem-1
DIVLTQSPDS LAVSLGERAT INCRASESVD TEDYSELHWY QQKPGQPPKL LIYRASNLES
GVPDRESGSG SGTDETLTIS SLQAEDVAVY YCQQSNEDPY TEGQGTKLEI K
SEQ ID NO: 22-VH of antagonist antibody against trem-1
EVQLVESGGG LVQPGGSLKL SCAASGFTES TYAMHWVRQA SGKGLEWVGR IRTKSSNYAT
YYAASVKGRF TISRDDSKNT AYLQMNSLKT EDTAVYYCTR DMGIRRQFAY WGQGTLVTVS
S
SEQ ID NO: 23-anti-CD86 antibody VL
DIQMTQSPAS ISASVGETVT ITCRASENIY SYLVWYQQKE GKTPHLLVYN AKTLAEGVPS
RFSGSGSGTQ FSLKINSLQP EDFGTYYCQH HYGTPLTFGA GTKLELK
SEQ ID NO: 24-anti-CD86 antibody VH
QVQLQQSGAE LARPGASVKL SCKASGFTFT DHFINWVRQR TGQGLEWIGE IYPGTGNAFY
SEKFKGKATL TADKSSSTAY MHLSSLTSED SAVEFCASPL RSGSHYWYFD VWGAGTTVTV
SS
SEQ ID NO: 25-CTLA-4 ECD abatacept (signal peptide is underlined)
MGVLLTQRTL LSLVLALLFP SMASMAMHVA QPAVVLASSR GIASFVCEYA SPGKATEVRV
TVLRQADSQV TEVCAATYMM GNELTELDDS ICTGTSSGNQ VNLTIQGLRA MDTGLYICKV
ELMYPPPYYL GIGNGTQIYV IDPEPCPDSD
SEQ ID NO: 26-dupilumab light chain
DIVMTQSPLS LPVTPGEPAS ISCRSSQSLL YSIGYNYLDW YLQKSGQSPQ LLIYLGSNRA
SGVPDRFSGS GSGTDFTLKI SRVEAEDVGF YYCMQALQTP YTFGQGTKLE IKRTVAAPSV
FIFPPSDEQL KSGTASVVCL LNNFYPREAK VQWKVDNALQ SGNSQESVTE QDSKDSTYSL
SSTLTLSKAD YEKHKVYACE VTHQGLSSPV TKSENRGEC
SEQ ID NO: 27-dupilumab heavy chain-IL10, knob
EVQLVESGGG LEQPGGSLRL SCAGSGFTER DYAMTWVRQA PGKGLEWVSS ISGSGGNTYY
ADSVKGRFTI SRDNSKNTLY LQMNSLRAED TAVYYCAKDR LSITIRPRYY GLDVWGQGTT
VTVSSASTKG PSVFPLAPCS RSTSESTAAL GCLVKDYFPE PVTVSWNSGA LTSGVHTEPA
VLQSSGLYSL SSVVTVPSSS LGTKTYTCNV DHKPSNTKVD KRVESKYGPP CPPCPAPEFL
GGPSVFLFPP KPKDTLYITR EPEVTCVVVD VSQEDPEVQF NWYVDGVEVH NAKTKPREEQ
FNSTYRVVSV LTVLHQDWLN GKEYKCKVSN KGLPSSIEKT ISKAKGQPRE PQVYTLPPCQ
EEMTKNQVSL WCLVKGFYPS DIAVEWESNG QPENNYKTTP PVLDSDGSFF LYSRLTVDKS
RWQEGNVFSC SVMHEALHNH YTQKSLSLSL GA(GGGGS)ₙ₁ SPGQGTQSEN SCTHEPGNLP
NMLRDLRDAF SRVKTFFQMK DQLDNLLLKE SLLEDEKGYL GCQALSEMIQ FYLEEVMPQA
ENQDPDIKAH VNSLGENLKT LRLRLRRCHR SLPCENKSKA VEQVKNAENK LQEKGIYKAM
SEFDIFINYI EAYMTMKIRN; wherein n1 = 0, 1, 2, or 3
SEQ ID NO: 28-dupilumab heavy chain-IL10, knob, v2
EVQLVESGGG LEQPGGSLRL SCAGSGFTER DYAMTWVRQA PGKGLEWVSS ISGSGGNTYY
ADSVKGRFTI SRDNSKNTLY LQMNSLRAED TAVYYCAKDR LSITIRPRYY GLDVWGQGTT
VTVSSASTKG PSVEPLAPCS RSTSESTAAL GCLVKDYFPE PVTVSWNSGA LTSGVHTEPA
VLQSSGLYSL SSVVTVPSSS LGTKTYTCNV DHKPSNTKVD KRVESKYGPP CPPCPAPEFL
GGPSVFLFPP KPKDTLYITR EPEVTCVVVD VSQEDPEVQF NWYVDGVEVH NAKTKPREEQ
FNSTYRVVSV LTVLHQDWLN GKEYKCKVSN KGLPSSIEKT ISKAKGQPRE PQVYTLPPCQ
EEMTKNQVSL WCLVKGFYPS DIAVEWESNG QPENNYKTTP PVLDSDGSFF LYSRLTVDKS
RWQEGNVFSC SVMHEALHNH YTQKSLSLSL GA(GGGGS)ₙ₁ SPGQGTQSEN SCTHEPGNLP
NMLRDLRDAF SRVKTFFQMK DQLDNLLLKE SLLEDFKGYL GCQALSEMIQ FYLEEVMPQA
ENQDPDIKAH VNSLGENLKT LRLRLRRCHR SLPCENKSKA VEQVKNAFNK LQEKGIYKAM
SEFDIFINYI EAYMTMKIRN (GGGGSAA)ₙ₂ (PYAYWMRG GGG)ₙ₃SPGQ GTQSENSCTH
FPGNLPNMLR DLRDAFSRVK TFFQMKDQLD NLLLKESLLE DEKGYLGCQA LSEMIQFYLE
EVMPQAENQD PDIKAHVNSL GENLKTLRLR LRRCHRSLPC ENKSKAVEQV KNAFNKLQEK
GIYKAMSEFD IFINYIEAYM TMKIRN; wherein n1 = 0, 1, 2, or 3; n2 = 1,
2, 3, 4, or 5; and n3 = 0, or 1.
SEQ ID NO: 29-dupilumab heavy chain-scFv1, hole
EVQLVESGGG LEQPGGSLRL SCAGSGFTER DYAMTWVRQA PGKGLEWVSS ISGSGGNTYY
ADSVKGRFTI SRDNSKNTLY LQMNSLRAED TAVYYCAKDR LSITIRPRYY GLDVWGQGTT
VTVSSASTKG PSVFPLAPCS RSTSESTAAL GCLVKDYFPE PVTVSWNSGA LTSGVHTFPA
VLQSSGLYSL SSVVTVPSSS LGTKTYTCNV DHKPSNTKVD KRVESKYGPP CPPCPAPEFL
GGPSVFLFPP KPKDTLYITR EPEVTCVVVD VSQEDPEVQF NWYVDGVEVH NAKTKPREEQ
FNSTYRVVSV LTVLHQDWLN GKEYKCKVSN KGLPSSIEKT ISKAKGQPRE PQVCTLPPSQ
EEMTKNQVSL SCAVKGFYPS DIAVEWESNG QPENNYKTTP PVLDSDGSFF LVSRLTVDKS
RWQEGNVFSC SVMHEALHNH YTQKSLSLSL GA(GGGGSAA)ₙ₁ DIQMTQSPS SLSASVGDRV
TITCKTSQNI FENLAWYQQK PGKAPKLLIY NASPLQAGVP SRESGSGSGT DETLTISSLQ
PEDFATYYCH QYYSGYTEGP GTKLELKGGG GSGGGGSGGG GSQVQLVESG GGVVQPGRSL
RLSCAASGFT FSDYHMAWVR QAPGKGLEWV ASITLDATYT YYRDSVRGRE TISRDNSKNT
LYLQMNSLRA EDTAVYYCAR HRGESVWLDY WGQGTLVTVS
S[(GGGGSAA)ₙ₂(PYAYWMR)ₙ₃(GGGGS)ₙ₄D IQMTQSPSSL SASVGDRVTI TCKTSQNIFE
NLAWYQQKPG KAPKLLIYNA SPLQAGVPSR FSGSGSGTDF TLTISSLQPE DFATYYCHQY
YSGYTFGPGT KLELKGGGGS GGGGSGGGGS QVQLVESGGG VVQPGRSLRL SCAASGETES
DYHMAWVRQA PGKGLEWVAS ITLDATYTYY RDSVRGRETI SRDNSKNTLY LQMNSLRAED
TAVYYCARHR GFSVWLDYWG QGTLVTVSS]ₙ₅; wherein n1 = 1, 2, 3, 4, or 5;
n2 = 1, 2, 3, 4, or 5; n3 = 0 or1; n4 = 0, 1, or 2, and n5 = 0,
or 1
SEQ ID NO: 30-dupilumab heavy chain-scFv2 hole
EVQLVESGGG LEQPGGSLRL SCAGSGFTER DYAMTWVRQA PGKGLEWVSS ISGSGGNTYY
ADSVKGRFTI SRDNSKNTLY LQMNSLRAED TAVYYCAKDR LSITIRPRYY GLDVWGQGTT
VTVSSASTKG PSVFPLAPCS RSTSESTAAL GCLVKDYFPE PVTVSWNSGA LTSGVHTFPA
VLQSSGLYSL SSVVTVPSSS LGTKTYTCNV DHKPSNTKVD KRVESKYGPP CPPCPAPEFL
GGPSVFLFPP KPKDTLYITR EPEVTCVVVD VSQEDPEVQF NWYVDGVEVH NAKTKPREEQ
FNSTYRVVSV LTVLHQDWLN GKEYKCKVSN KGLPSSIEKT ISKAKGQPRE PQVCTLPPSQ
EEMTKNQVSL SCAVKGFYPS DIAVEWESNG QPENNYKTTP PVLDSDGSFF LVSRLTVDKS
RWQEGNVFSC SVMHEALHNH YTQKSLSLSL GA(GGGGSAA)ₙ₁ QVQLVESGG GVVQPGRSLR
LSCAASGFTF SDYHMAWVRQ APGKGLEWVA SITLDATYTY YRDSVRGRFT ISRDNSKNTL
YLQMNSLRAE DTAVYYCARH RGFSVWLDYW GQGTLVTVSS GGGGSGGGGS GGGGSDIQMT
QSPSSLSASV GDRVTITCKT SQNIFENLAW YQQKPGKAPK LLIYNASPLQ AGVPSRESGS
GSGTDFTLTI SSLQPEDFAT YYCHQYYSGY TFGPGTKLEL
K[(GGGGSAA)ₙ₂(PYAYWMR)ₙ₃(GGGGS)ₙ₄Q VQLVESGGGV VQPGRSLRLS CAASGFTESD
YHMAWVRQAP GKGLEWVASI TLDATYTYYR DSVRGRETIS RDNSKNTLYL QMNSLRAEDT
AVYYCARHRG FSVWLDYWGQ GTLVTVSSGG GGSGGGGSGG GGSDIQMTQS PSSLSASVGD
RVTITCKTSQ NIFENLAWYQ QKPGKAPKLL IYNASPLQAG VPSRESGSGS GTDFTLTISS
LQPEDFATYY CHQYYSGYTF GPGTKLELK]ₙ₅; wherein n1 = 1, 2, 3, 4, or 5;
n2 = 1, 2, 3, 4, or 5; n3 = 0 or 1; n4 = 0, 1, or 2, and n5 = 0,
or 1.
SEQ ID NO: 31-dupilumab heavy
EVQLVESGGG LEQPGGSLRL SCAGSGFTER DYAMTWVRQA PGKGLEWVSS ISGSGGNTYY
ADSVKGRFTI SRDNSKNTLY LQMNSLRAED TAVYYCAKDR LSITIRPRYY GLDVWGQGTT
VTVSSASTKG PSVFPLAPCS RSTSESTAAL GCLVKDYFPE PVTVSWNSGA LTSGVHTFPA
VLQSSGLYSL SSVVTVPSSS LGTKTYTCNV DHKPSNTKVD KRVESKYGPP CPPCPAPEFL
GGPSVFLFPP KPKDTLMISR TPEVTCVVVD VSQEDPEVQF NWYVDGVEVH NAKTKPREEQ
FNSTYRVVSV LTVLHQDWLN GKEYKCKVSN KGLPSSIEKT ISKAKGQPRE PQVYTLPPSQ
EEMTKNQVSL TCLVKGFYPS DIAVEWESNG QPENNYKTTP PVLDSDGSFF LYSRLTVDKS
RWQEGNVFSC SVMHEALHNH YTQKSLSLSL GA(GGGGSAA)ₙ₁ SPGQGTQSE NSCTHEPGNL
PNMLRDLRDA FSRVKTFFQM KDQLDNLLLK ESLLEDEKGY LGCQALSEMI QFYLEEVMPQ
AENQDPDIKA HVNSLGENLK TLRLRLRRCH RSLPCENKSK AVEQVKNAFN KLQEKGIYKA
MSEFDIFINY IEAYMTMKIR N(GGGGSAA)ₙ₂(PYAYWMR)ₙ₃(GGGGS)ₙ₄DI QMTQSPSSLS
ASVGDRVTIT CKTSQNIFEN LAWYQQKPGK APKLLIYNAS PLQAGVPSRF SGSGSGTDFT
LTISSLQPED FATYYCHQYY SGYTFGPGTK LELKGGGGSG GGGSGGGGSQ VQLVESGGGV
VQPGRSLRLS CAASGFTESD YHMAWVRQAP GKGLEWVASI TLDATYTYYR DSVRGRETIS
RDNSKNTLYL QMNSLRAEDT AVYYCARHRG FSVWLDYWGQ GTLVTVSS; wherein
n1 = 0, 1, 2, 3, 4, or 5; n2 = 0, 1, 2, 3, 4, or 5; n3 = 0 or 1;
and n4 = 0, 1, or 2.
SEQ ID NO: 32-dupilumab heavy chain - scFv1-IL10-homodimer 2
EVQLVESGGG LEQPGGSLRL SCAGSGFTER DYAMTWVRQA PGKGLEWVSS ISGSGGNTYY
ADSVKGRFTI SRDNSKNTLY LQMNSLRAED TAVYYCAKDR LSITIRPRYY GLDVWGQGTT
VTVSSASTKG PSVFPLAPCS RSTSESTAAL GCLVKDYFPE PVTVSWNSGA LTSGVHTFPA
VLQSSGLYSL SSVVTVPSSS LGTKTYTCNV DHKPSNTKVD KRVESKYGPP CPPCPAPEFL
GGPSVFLFPP KPKDTLMISR TPEVTCVVVD VSQEDPEVQF NWYVDGVEVH NAKTKPREEQ
FNSTYRVVSV LTVLHQDWLN GKEYKCKVSN KGLPSSIEKT ISKAKGQPRE PQVYTLPPSQ
EEMTKNQVSL TCLVKGFYPS DIAVEWESNG QPENNYKTTP PVLDSDGSFF LYSRLTVDKS
RWQEGNVFSC SVMHEALHNH YTQKSLSLSL GA(GGGGS)ₙ₁DIQMTQSPSSL SASVGDRVTI
TCKTSQNIFE NLAWYQQKPG KAPKLLIYNA SPLQAGVPSR FSGSGSGTDE TLTISSLQPE
DFATYYCHQY YSGYTFGPGT KLELKGGGGS GGGGSGGGGS QVQLVESGGG VVQPGRSLRL
SCAASGFTES DYHMAWVRQA PGKGLEWVAS ITLDATYTYY RDSVRGRFTI SRDNSKNTLY
LQMNSLRAED TAVYYCARHR GFSVWLDYWG QGTLVTVSS (GGGGSAA)ₙ₂PY AYWMRGGGGS
PGQGTQSENS CTHEPGNLPN MLRDLRDAFS RVKTFFQMKD QLDNLLLKES LLEDEKGYLG
CQALSEMIQF YLEEVMPQAE NQDPDIKAHV NSLGENLKTL RLRLRRCHRS LPCENKSKAV
EQVKNAFNKL QEKGIYKAMS EFDIFINYIE AYMTMKIRN; wherein n1 = 0, 1, 2,
3, 4, or 5; and n2 = 0, 1, 2, 3, 4, or 5.
SEQ ID NO: 33-dupilumab heavy chain-scFv2-IL10-homodimer 3
EVQLVESGGG LEQPGGSLRL SCAGSGFTER DYAMTWVRQA PGKGLEWVSS ISGSGGNTYY
ADSVKGRFTI SRDNSKNTLY LQMNSLRAED TAVYYCAKDR LSITIRPRYY GLDVWGQGTT
VTVSSASTKG PSVFPLAPCS RSTSESTAAL GCLVKDYFPE PVTVSWNSGA LTSGVHTFPA
VLQSSGLYSL SSVVTVPSSS LGTKTYTCNV DHKPSNTKVD KRVESKYGPP CPPCPAPEFL
GGPSVFLFPP KPKDTLMISR TPEVTCVVVD VSQEDPEVQF NWYVDGVEVH NAKTKPREEQ
FNSTYRVVSV LTVLHQDWLN GKEYKCKVSN KGLPSSIEKT ISKAKGQPRE PQVYTLPPSQ
EEMTKNQVSL TCLVKGFYPS DIAVEWESNG QPENNYKTTP PVLDSDGSFF LYSRLTVDKS
RWQEGNVESC SVMHEALHNH YTQKSLSLSL GA(GGGGSAA)ₙ₁SPGQGTQSE NSCTHEPGNL
PNMLRDLRDA FSRVKTFFQM KDQLDNLLLK ESLLEDFKGY LGCQALSEMI QFYLEEVMPQ
AENQDPDIKA HVNSLGENLK TLRLRLRRCH RSLPCENKSK AVEQVKNAFN KLQEKGIYKA
MSEFDIFINY IEAYMTMKIR N(GGGGSAA)ₙ₂(PYAYWMR)ₙ₃(GGGGS)ₙ₄Q VQLVESGGGV
VQPGRSLRLS CAASGFTESD YHMAWVRQAP GKGLEWVASI TLDATYTYYR DSVRGRATIS
RDNSKNTLYL QMNSLRAEDT AVYYCARHRG FSVWLDYWGQ GTLVTVSSGG GGSGGGGSGG
GGSDIQMTQS PSSLSASVGD RVTITCKTSQ NIFENLAWYQ QKPGKAPKLL IYNASPLQAG
VPSRESGSGS GTDFTLTISS LQPEDFATYY CHQYYSGYTF GPGTKLELK; wherein n1 =
0, 1, 2, 3, 4, or 5; n2 = 1, 2, 3, 4, or 5; n3 = 0, or 1;
n4 = 0, 1, or 2.
SEQ ID NO: 34-dupilumab heavy chain-scFv2-IL10-homodimer 4
EVQLVESGGG LEQPGGSLRL SCAGSGFTER DYAMTWVRQA PGKGLEWVSS ISGSGGNTYY
ADSVKGRFTI SRDNSKNTLY LQMNSLRAED TAVYYCAKDR LSITIRPRYY GLDVWGQGTT
VTVSSASTKG PSVEPLAPCS RSTSESTAAL GCLVKDYFPE PVTVSWNSGA LTSGVHTFPA
VLQSSGLYSL SSVVTVPSSS LGTKTYTCNV DHKPSNTKVD KRVESKYGPP CPPCPAPEFL
GGPSVFLFPP KPKDTLMISR TPEVTCVVVD VSQEDPEVQF NWYVDGVEVH NAKTKPREEQ
FNSTYRVVSV LTVLHQDWLN GKEYKCKVSN KGLPSSIEKT ISKAKGQPRE PQVYTLPPSQ
EEMTKNQVSL TCLVKGFYPS DIAVEWESNG QPENNYKTTP PVLDSDGSFF LYSRLTVDKS
RWQEGNVESC SVMHEALHNH YTQKSLSLSL GA(GGGGSAA)ₙ₁QVQLVESGG GVVQPGRSLR
LSCAASGFTF SDYHMAWVRQ APGKGLEWVA SITLDATYTY YRDSVRGRET ISRDNSKNTL
YLQMNSLRAE DTAVYYCARH RGFSVWLDYW GQGTLVTVSS GGGGSGGGGS GGGGSDIQMT
QSPSSLSASV GDRVTITCKT SQNIFENLAW YQQKPGKAPK LLIYNASPLQ AGVPSRESGS
GSGTDFTLTI SSLQPEDFAT YYCHQYYSGY TFGPGTKLEL K(GGGGSAA)ₙ₂PYAYWMRGGG
GSPGQGTQSE NSCTHEPGNL PNMLRDLRDA FSRVKTFFQM KDQLDNLLLK ESLLEDEKGY
LGCQALSEMI QFYLEEVMPQ AENQDPDIKA HVNSLGENLK TLRLRLRRCH RSLPCENKSK
AVEQVKNAFN KLQEKGIYKA MSEFDIFINY IEAYMTMKIR N; wherein n1 = 0, 1,
2, 3, 4, or 5; n2 = 1, 2, 3, 4, or 5
SEQ ID NO: 35-IL-5 antibody benralizumab Light chain
DIQMTQSPSS LSASVGDRVT ITCGTSEDII NYLNWYQQKP GKAPKLLIYH TSRLQSGVPS
RFSGSGSGTD FTLTISSLQP EDFATYYCQQ GYTLPYTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSEN RGEC
SEQ ID NO: 36-IL-5 antibody benralizumab heavy chain-IL-10 knob
EVQLVQSGAE VKKPGASVKV SCKASGYTFT SYVIHWVRQR PGQGLAWMGY INPYNDGTKY
NERFKGKVTI TSDRSTSTVY MELSSLRSED TAVYLCGREG IRYYGLLGDY WGQGTLVTVS
SASTKGPSVF PLAPCSRSTS ESTAALGCLV KDYFPEPVTV SWNSGALTSG VHTFPAVLQS
SGLYSLSSVV TVPSSSLGTK TYTCNVDHKP SNTKVDKRVE SKYGPPCPPC PAPEFLGGPS
VFLFPPKPKD TLYITREPEV TCVVVDVSQE DPEVQENWYV DGVEVHNAKT KPREEQENST
YRVVSVLTVL HQDWLNGKEY KCKVSNKGLP SSIEKTISKA KGQPREPQVY TLPPCQEEMT
KNQVSLWCLV KGFYPSDIAV EWESNGQPEN NYKTTPPVLD SDGSFFLYSR LTVDKSRWQE
GNVFSCSVMH EALHNHYTQK SLSLSLGA(GGGGS)ₙ₁SPGQG TQSENSCTHE PGNLPNMLRD
LRDAFSRVKT FFQMKDQLDN LLLKESLLED FKGYLGCQAL SEMIQFYLEE VMPQAENQDP
DIKAHVNSLG ENLKTLRLRL RRCHRSLPCE NKSKAVEQVK NAFNKLQEKG IYKAMSEFDI
FINYIEAYMT MKIRN[(GGGGSAA)ₙ₂SPGQG TQSENSCTHE PGNLPNMLRD LRDAFSRVKT
FFQMKDQLDN LLLKESLLED FKGYLGCQAL SEMIQFYLEE VMPQAENQDP DIKAHVNSLG
ENLKTLRLRL RRCHRSLPCE NKSKAVEQVK NAFNKLQEKG IYKAMSEFDI FINYIEAYMT
MKIRN]ₙ₃; wherein n1 = 0, 1, 2, or 3; n2 = 1, 2, 3, 4, or 5; and
n3 = 0, or 1.
SEQ ID NO: 37-IL-5 antibody benralizumab heavy chain-scFv1, hole
EVQLVQSGAE VKKPGASVKV SCKASGYTFT SYVIHWVRQR PGQGLAWMGY INPYNDGTKY
NERFKGKVTI TSDRSTSTVY MELSSLRSED TAVYLCGREG IRYYGLLGDY WGQGTLVTVS
STVPSSSLGT KTYTCNVDHK PSNTKVDKRV ESKYGPPCPP CPAPEFLGGP SVELFPPKPK
DTLYITREPE VTCVVVDVSQ EDPEVQFNWY VDGVEVHNAK TKPREEQENS TYRVVSVLTV
LHQDWLNGKE YKCKVSNKGL PSSIEKTISK AKGQPREPQV CTLPPSQEEM TKNQVSLSCA
VKGFYPSDIA VEWESNGQPE NNYKTTPPVL DSDGSFFLVS RLTVDKSRWQ EGNVESCSVM
HEALHNHYTQ KSLSLSLGA (GGGGSAA)ₙ₁DI QMTQSPSSLS ASVGDRVTIT CKTSQNIFEN
LAWYQQKPGK APKLLIYNAS PLQAGVPSRF SGSGSGTDFT LTISSLQPED FATYYCHQYY
SGYTFGPGTK LELKGGGGSG GGGSGGGGSQ VQLVESGGGV VQPGRSLRLS CAASGFTESD
YHMAWVRQAP GKGLEWVASI TLDATYTYYR DSVRGRETIS RDNSKNTLYL QMNSLRAEDT
AVYYCARHRG FSVWLDYWGQ GTLVTVSS[(GGGGSAA)ₙ₂(GPLGVR)ₙ₃(GGGGS)ₙ₄DIQMTQ
SPSSLSASVG DRVTITCKTS QNIFENLAWY QQKPGKAPKL LIYNASPLQA GVPSRESGSG
SGTDETLTIS SLQPEDFATY YCHQYYSGYT FGPGTKLELK GGGGSGGGGS GGGGSQVQLV
ESGGGVVQPG RSLRLSCAAS GFTFSDYHMA WVRQAPGKGL EWVASITLDA TYTYYRDSVR
GRFTISRDNS KNTLYLQMNS LRAEDTAVYY CARHRGESVW LDYWGQGTLV TVSS]ₙ₅;
wherein n1 = 1, 2, 3, or 4; n2 = 1, 2, 3, 4, or 5; n3 = 0 or 1;
n4 = 0, 1, or 2; and n5 = 0, or 1.
SEQ ID NO: 38-IL-5 antibody benralizumab heavy chain-scFv2, hole
EVQLVQSGAE VKKPGASVKV SCKASGYTFT SYVIHWVRQR PGQGLAWMGY INPYNDGTKY
NERFKGKVTI TSDRSTSTVY MELSSLRSED TAVYLCGREG IRYYGLLGDY WGQGTLVTVS
SASTKGPSVF PLAPCSRSTS ESTAALGCLV KDYFPEPVTV SWNSGALTSG VHTEPAVLQS
SGLYSLSSVV TVPSSSLGTK TYTCNVDHKP SNTKVDKRVE SKYGPPCPPC PAPEFLGGPS
VFLFPPKPKD TLYITREPEV TCVVVDVSQE DPEVQENWYV DGVEVHNAKT KPREEQENST
YRVVSVLTVL HQDWLNGKEY KCKVSNKGLP SSIEKTISKA KGQPREPQVC TLPPSQEEMT
KNQVSLSCAV KGFYPSDIAV EWESNGQPEN NYKTTPPVLD SDGSFFLVSR LTVDKSRWQE
GNVFSCSVMH EALHNHYTQK SLSLSLGA (GGGGSAA)ₙ₁QVQ LVESGGGVVQ PGRSLRLSCA
ASGFTFSDYH MAWVRQAPGK GLEWVASITL DATYTYYRDS VRGRFTISRD NSKNTLYLQM
NSLRAEDTAV YYCARHRGES VWLDYWGQGT LVTVSSGGGG SGGGGSGGGG SDIQMTQSPS
SLSASVGDRV TITCKTSQNI FENLAWYQQK PGKAPKLLIY NASPLQAGVP SRESGSGSGT
DETLTISSLQ PEDFATYYCH QYYSGYTEGP
GTKLELK[(GGGGSAA)ₙ₂(GPLGVR)ₙ₃(GGGGS)ₙ₄QVQLVES GGGVVQPGRS LRLSCAASGE
TFSDYHMAWV RQAPGKGLEW VASITLDATY TYYRDSVRGR FTISRDNSKN TLYLQMNSLR
AEDTAVYYCA RHRGFSVWLD YWGQGTLVTV SSGGGGSGGG GSGGGGSDIQ MTQSPSSLSA
SVGDRVTITC KTSQNIFENL AWYQQKPGKA PKLLIYNASP LQAGVPSRES GSGSGTDETL
TISSLQPEDF ATYYCHQYYS GYTFGPGTKL ELK]ₙ₅; wherein n1 = 1, 2, 3, or
4; n2 = 1, 2, 3, 4, or 5; n3 = 0 or 1; n4 = 0, 1, or 2; and n5 =
0, or 1.
SEQ ID NO: 39-IL-5 antibody benralizumab heavy chain homodimer 1
EVQLVQSGAE VKKPGASVKV SCKASGYTFT SYVIHWVRQR PGQGLAWMGY INPYNDGTKY
NERFKGKVTI TSDRSTSTVY MELSSLRSED TAVYLCGREG IRYYGLLGDY WGQGTLVTVS
SASTKGPSVF PLAPCSRSTS ESTAALGCLV KDYFPEPVTV SWNSGALTSG VHTFPAVLQS
SGLYSLSSVV TVPSSSLGTK TYTCNVDHKP SNTKVDKRVE SKYGPPCPPC PAPEFLGGPS
VFLFPPKPKD TLMISRTPEV TCVVVDVSQE DPEVQFNWYV DGVEVHNAKT KPREEQENST
YRVVSVLTVL HQDWLNGKEY KCKVSNKGLP SSIEKTISKA KGQPREPQVY TLPPSQEEMT
KNQVSLTCLV KGFYPSDIAV EWESNGQPEN NYKTTPPVLD SDGSFFLYSR LTVDKSRWQE
GNVFSCSVMH EALHNHYTQK SLSLSLGA(GGGGSAA)ₙ₁SPG QGTQSENSCT HFPGNLPNML
RDLRDAFSRV KTFFQMKDQL DNLLLKESLL EDFKGYLGCQ ALSEMIQFYL EEVMPQAENQ
DPDIKAHVNS LGENLKTLRL RLRRCHRSLP CENKSKAVEQ VKNAFNKLQE KGIYKAMSEF
DIFINYIEAY MTMKIRN(GGGGSAA)ₙ₂(PYAYWMR)ₙ₃(GGGGS)ₙ₄DIQMTQS PSSLSASVGD
RVTITCKTSQ NIFENLAWYQ QKPGKAPKLL IYNASPLQAG VPSRESGSGS GTDETLTISS
LQPEDFATYY CHQYYSGYTF GPGTKLELKG GGGSGGGGSG GGGSQVQLVE SGGGVVQPGR
SLRLSCAASG FTESDYHMAW VRQAPGKGLE WVASITLDAT YTYYRDSVRG RFTISRDNSK
NTLYLQMNSL RAEDTAVYYC ARHRGFSVWL DYWGQGTLVT VSS ; wherein n1 = 0,
1, 2, 3, 4, or 5; n2 = 0, 1, 2, 3, 4, or 5; n3 = 0 or 1; and n4 =
0, 1, or 2.
SEQ ID NO: 40-IL-5 antibody benralizumab Heavy chain Homodimer 2
EVQLVQSGAE VKKPGASVKV SCKASGYTFT SYVIHWVRQR PGQGLAWMGY INPYNDGTKY
NERFKGKVTI TSDRSTSTVY MELSSLRSED TAVYLCGREG IRYYGLLGDY WGQGTLVTVS
SASTKGPSVF PLAPCSRSTS ESTAALGCLV KDYFPEPVTV SWNSGALTSG VHTFPAVLQS
SGLYSLSSVV TVPSSSLGTK TYTCNVDHKP SNTKVDKRVE SKYGPPCPPC PAPEFLGGPS
VFLFPPKPKD TLMISRTPEV TCVVVDVSQE DPEVQENWYV DGVEVHNAKT KPREEQFNST
YRVVSVLTVL HQDWLNGKEY KCKVSNKGLP SSIEKTISKA KGQPREPQVY TLPPSQEEMT
KNQVSLTCLV KGFYPSDIAV EWESNGQPEN NYKTTPPVLD SDGSFFLYSR LTVDKSRWQE
GNVFSCSVMH EALHNHYTQK SLSLSLGA(GGGGS)ₙ₁DIQMT QSPSSLSASV GDRVTITCKT
SQNIFENLAW YQQKPGKAPK LLIYNASPLQ AGVPSRESGS GSGTDETLTI SSLQPEDFAT
YYCHQYYSGY TFGPGTKLEL KGGGGSGGGG SGGGGSQVQL VESGGGVVQP GRSLRLSCAA
SGFTESDYHM AWVRQAPGKG LEWVASITLD ATYTYYRDSV RGRFTISRDN SKNTLYLQMN
SLRAEDTAVY YCARHRGFSV WLDYWGQGTL VTVSS(GGGGSAA)ₙ₂SPGQGT QSENSCTHEP
GNLPNMLRDL RDAFSRVKTF FQMKDQLDNL LLKESLLEDF KGYLGCQALS EMIQFYLEEV
MPQAENQDPD IKAHVNSLGE NLKTLRLRLR RCHRSLPCEN KSKAVEQVKN AFNKLQEKGI
YKAMSEFDIF INYIEAYMTM KIRN; wherein n1 = 0, 1, 2, 3, 4, or 5; and
n2 = 0, 1, 2, 3, 4, or 5.
SEQ ID NO: 41-IL-5 antibody benralizumab heavy chain homodimer 3
EVQLVQSGAE VKKPGASVKV SCKASGYTFT SYVIHWVRQR PGQGLAWMGY INPYNDGTKY
NERFKGKVTI TSDRSTSTVY MELSSLRSED TAVYLCGREG IRYYGLLGDY WGQGTLVTVS
SASTKGPSVF PLAPCSRSTS ESTAALGCLV KDYFPEPVTV SWNSGALTSG VHTFPAVLQS
SGLYSLSSVV TVPSSSLGTK TYTCNVDHKP SNTKVDKRVE SKYGPPCPPC PAPEFLGGPS
VFLFPPKPKD TLMISRTPEV TCVVVDVSQE DPEVQFNWYV DGVEVHNAKT KPREEQENST
YRVVSVLTVL HQDWLNGKEY KCKVSNKGLP SSIEKTISKA KGQPREPQVY TLPPSQEEMT
KNQVSLTCLV KGFYPSDIAV EWESNGQPEN NYKTTPPVLD SDGSFFLYSR LTVDKSRWQE
GNVFSCSVMH EALHNHYTQK SLSLSLGA(GGGGSAA)ₙ₁SP GQGTQSENSC THEPGNLPNM
LRDLRDAFSR VKTFFQMKDQ LDNLLLKESL LEDFKGYLGC QALSEMIQFY LEEVMPQAEN
QDPDIKAHVN SLGENLKTLR LRLRRCHRSL PCENKSKAVE QVKNAFNKLQ EKGIYKAMSE
EDIFINYIEA YMTMKIRN(GGGGSAA)ₙ₂(PYAYWMR)ₙ₃(GGGGS)ₙ₄QVQLVE SGGGVVQPGR
SLRLSCAASG FTESDYHMAW VRQAPGKGLE WVASITLDAT YTYYRDSVRG RFTISRDNSK
NTLYLQMNSL RAEDTAVYYC ARHRGFSVWL DYWGQGTLVT VSSGGGGSGG GGSGGGGSDI
QMTQSPSSLS ASVGDRVTIT CKTSQNIFEN LAWYQQKPGK APKLLIYNAS PLQAGVPSRE
SGSGSGTDFT LTISSLQPED FATYYCHQYY SGYTFGPGTK LELK; wherein n1 = 0,
1, 2, 3, 4, or 5; n2 = 1, 2, 3, 4, or 5; n3 = 0 or 1; and n4 = 0,
1, or 2
SEQ ID NO: 42-IL-5 antibody benralizumab heavy chain homodimer 4
EVQLVQSGAE VKKPGASVKV SCKASGYTFT SYVIHWVRQR PGQGLAWMGY INPYNDGTKY
NERFKGKVTI TSDRSTSTVY MELSSLRSED TAVYLCGREG IRYYGLLGDY WGQGTLVTVS
SASTKGPSVF PLAPCSRSTS ESTAALGCLV KDYFPEPVTV SWNSGALTSG VHTFPAVLQS
SGLYSLSSVV TVPSSSLGTK TYTCNVDHKP SNTKVDKRVE SKYGPPCPPC PAPEFLGGPS
VFLFPPKPKD TLMISRTPEV TCVVVDVSQE DPEVQFNWYV DGVEVHNAKT KPREEQFNST
YRVVSVLTVL HQDWLNGKEY KCKVSNKGLP SSIEKTISKA KGQPREPQVY TLPPSQEEMT
KNQVSLTCLV KGFYPSDIAV EWESNGQPEN NYKTTPPVLD SDGSFFLYSR LTVDKSRWQE
GNVFSCSVMH EALHNHYTQK SLSLSLGA(GGGGSAA)ₙ₁QVQ LVESGGGVVQ PGRSLRLSCA
ASGFTFSDYH MAWVRQAPGK GLEWVASITL DATYTYYRDS VRGRFTISRD NSKNTLYLQM
NSLRAEDTAV YYCARHRGES VWLDYWGQGT LVTVSSGGGG SGGGGSGGGG SDIQMTQSPS
SLSASVGDRV TITCKTSQNI FENLAWYQQK PGKAPKLLIY NASPLQAGVP SRESGSGSGT
DETLTISSLQ PEDFATYYCH QYYSGYTFGP GTKLELK(GGGGSAA)ₙ₂SPGQ GTQSENSCTH
FPGNLPNMLR DLRDAFSRVK TFFQMKDQLD NLLLKESLLE DEKGYLGCQA LSEMIQFYLE
EVMPQAENQD PDIKAHVNSL GENLKTLRLR LRRCHRSLPC ENKSKAVEQV KNAFNKLQEK
GIYKAMSEED IFINYIEAYM TMKIRN; wherein n1 = 0, 1, 2, 3, 4, or 5;
n2 = 1, 2, 3, 4, or 5
SEQ ID NO: 43-sarilumab anti-IL-6 receptor antibody light chain
DIQMTQSPSS VSASVGDRVT ITCRASQGIS SWLAWYQQKP GKAPKLLIYG ASSLESGVPS
RFSGSGSGTD FTLTISSLQP EDFASYYCQQ ANSFPYTFGQ GTKLEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSEN RGEC
SEQ ID NO: 44-sarilumab anti-IL-6 receptor-IL10 knob
EVQLVESGGG LVQPGRSLRL SCAASRFTED DYAMHWVRQA PGKGLEWVSG ISWNSGRIGY
ADSVKGRFTI SRDNAENSLF LQMNGLRAED TALYYCAKGR DSFDIWGQGT MVTVSSASTK
GPSVFPLAPC SRSTSESTAA LGCLVKDYFP EPVTVSWNSG ALTSGVHTEP AVLQSSGLYS
LSSVVTVPSS SLGTKTYTCN VDHKPSNTKV DKRVESKYGP PCPPCPAPEF LGGPSVFLFP
PKPKDTLYIT REPEVTCVVV DVSQEDPEVQ FNWYVDGVEV HNAKTKPREE QFNSTYRVVS
VLTVLHQDWL NGKEYKCKVS NKGLPSSIEK TISKAKGQPR EPQVYTLPPC QEEMTKNQVS
LWCLVKGFYP SDIAVEWESN GQPENNYKTT PPVLDSDGSF FLYSRLTVDK SRWQEGNVES
CSVMHEALHN HYTQKSLSLS LGA(GGGGS)ₙ₁SPGQGTQSEN SCTHFPGNLP NMLRDLRDAF
SRVKTFFQMK DQLDNLLLKE SLLEDFKGYL GCQALSEMIQ FYLEEVMPQA ENQDPDIKAH
VNSLGENLKT LRLRLRRCHR SLPCENKSKA VEQVKNAENK LQEKGIYKAM SEFDIFINYI
EAYMTMKIRN [(GGGGSAA)ₙ₂SPGQGTQSEN SCTHFPGNLP NMLRDLRDAF SRVKTFFQMK
DQLDNLLLKE SLLEDEKGYL GCQALSEMIQ FYLEEVMPQA ENQDPDIKAH VNSLGENLKT
LRLRLRRCHR SLPCENKSKA VEQVKNAFNK LQEKGIYKAM SEFDIFINYI EAYMTMKIRN]ₙ₃;
wherein n1 = 0, 1, 2, or 3; n2 = 1, 2, 3, 4, or 5; and n3 = 0
or 1.
SEQ ID NO: 45-sarilumab anti-IL-6 receptor-IL10 scFv1 hole
EVQLVESGGG LVQPGRSLRL SCAASRFTED DYAMHWVRQA PGKGLEWVSG ISWNSGRIGY
ADSVKGRFTI SRDNAENSLF LQMNGLRAED TALYYCAKGR DSFDIWGQGT MVTVSSASTK
GPSVFPLAPC SRSTSESTAA LGCLVKDYFP EPVTVSWNSG ALTSGVHTEP AVLQSSGLYS
LSSVVTVPSS SLGTKTYTCN VDHKPSNTKV DKRVESKYGP PCPPCPAPEF LGGPSVELEP
PKPKDTLYIT REPEVTCVVV DVSQEDPEVQ FNWYVDGVEV HNAKTKPREE QFNSTYRVVS
VLTVLHQDWL NGKEYKCKVS NKGLPSSIEK TISKAKGQPR EPQVCTLPPS QEEMTKNQVS
LSCAVKGFYP SDIAVEWESN GQPENNYKTT PPVLDSDGSF FLVSRLTVDK SRWQEGNVES
CSVMHEALHN HYTQKSLSLS LGA(GGGGSAA)ₙ₁DIQMTQSP SSLSASVGDR VTITCKTSQN
IFENLAWYQQ KPGKAPKLLI YNASPLQAGV PSRESGSGSG TDFTLTISSL QPEDFATYYC
HQYYSGYTFG PGTKLELKGG GGSGGGGSGG GGSQVQLVES GGGVVQPGRS LRLSCAASGE
TFSDYHMAWV RQAPGKGLEW VASITLDATY TYYRDSVRGR FTISRDNSKN TLYLQMNSLR
AEDTAVYYCA RHRGESVWLD YWGQGTLVTV
SS[(GGGGSAA)ₙ₂(PYAYWMR)ₙ₃(GGGGS)ₙ₄DIQMTQSPSSL SASVGDRVTI TCKTSQNIFE
NLAWYQQKPG KAPKLLIYNA SPLQAGVPSR FSGSGSGTDE TLTISSLQPE DFATYYCHQY
YSGYTFGPGT KLELKGGGGS GGGGSGGGGS QVQLVESGGG VVQPGRSLRL SCAASGFTES
DYHMAWVRQA PGKGLEWVAS ITLDATYTYY RDSVRGRFTI SRDNSKNTLY LQMNSLRAED
TAVYYCARHR GFSVWLDYWG QGTLVTVSS]ₙ₅; wherein n1 = 1, 2, 3, or 4; n2 =
1, 2, 3, 4, or 5; n3 = 0 or 1; n4 = 0, 1, or 2; and n5 = 0, or
1.
SEQ ID NO: 46-sarilumab anti-IL-6 receptor-IL10 scFv2 hole
EVQLVESGGG LVQPGRSLRL SCAASRFTED DYAMHWVRQA PGKGLEWVSG ISWNSGRIGY
ADSVKGRFTI SRDNAENSLF LQMNGLRAED TALYYCAKGR DSFDIWGQGT MVTVSSASTK
GPSVFPLAPC SRSTSESTAA LGCLVKDYFP EPVTVSWNSG ALTSGVHTEP AVLQSSGLYS
LSSVVTVPSS SLGTKTYTCN VDHKPSNTKV DKRVESKYGP PCPPCPAPEF LGGPSVELFP
PKPKDTLYIT REPEVTCVVV DVSQEDPEVQ FNWYVDGVEV HNAKTKPREE QFNSTYRVVS
VLTVLHQDWL NGKEYKCKVS NKGLPSSIEK TISKAKGQPR EPQVCTLPPS QEEMTKNQVS
LSCAVKGFYP SDIAVEWESN GQPENNYKTT PPVLDSDGSF FLVSRLTVDK SRWQEGNVES
CSVMHEALHN HYTQKSLSLS LGA(GGGGSAA)ₙ₁QVQLVESG GGVVQPGRSL RLSCAASGET
FSDYHMAWVR QAPGKGLEWV ASITLDATYT YYRDSVRGRF TISRDNSKNT LYLQMNSLRA
EDTAVYYCAR HRGFSVWLDY WGQGTLVTVS SGGGGSGGGG SGGGGSDIQM TQSPSSLSAS
VGDRVTITCK TSQNIFENLA WYQQKPGKAP KLLIYNASPL QAGVPSRESG SGSGTDFTLT
ISSLQPEDFA TYYCHQYYSG YTFGPGTKLE LK[(GGGGSAA)ₙ₂(PYAYWMR)ₙ₃(GGGGS)ₙ₄
QVQLVESGGG VVQPGRSLRL SCAASGFTES DYHMAWVRQA PGKGLEWVAS ITLDATYTYY
RDSVRGRFTI SRDNSKNTLY LQMNSLRAED TAVYYCARHR GESVWLDYWG QGTLVTVSSG
GGGSGGGGSG GGGSDIQMTQ SPSSLSASVG DRVTITCKTS QNIFENLAWY QQKPGKAPKL
LIYNASPLQA GVPSRESGSG SGTDETLTIS SLQPEDFATY YCHQYYSGYT FGPGTKLELK]
n5; Wherein n1 = 1, 2, 3, or 4; n2 = 1, 2, 3, 4, or 5; n3 = 0 or 1;
n4 = 0, 1, or 2; and n5 = 0, or 1.
SEQ ID NO: 47-sarilumab anti-IL-6 receptor-IL10 homodimer 1
EVQLVESGGG LVQPGRSLRL SCAASRFTED DYAMHWVRQA PGKGLEWVSG ISWNSGRIGY
ADSVKGRFTI SRDNAENSLF LQMNGLRAED TALYYCAKGR DSFDIWGQGT MVTVSSASTK
GPSVFPLAPC SRSTSESTAA LGCLVKDYFP EPVTVSWNSG ALTSGVHTFP AVLQSSGLYS
LSSVVTVPSS SLGTKTYTCN VDHKPSNTKV DKRVESKYGP PCPPCPAPEF LGGPSVELFP
PKPKDTLMIS RTPEVTCVVV DVSQEDPEVQ FNWYVDGVEV HNAKTKPREE QFNSTYRVVS
VLTVLHQDWL NGKEYKCKVS NKGLPSSIEK TISKAKGQPR EPQVYTLPPS QEEMTKNQVS
LTCLVKGFYP SDIAVEWESN GQPENNYKTT PPVLDSDGSF FLYSRLTVDK SRWQEGNVES
CSVMHEALHN HYTQKSLSLS LGA(GGGGSAA)ₙ₁SPGQGTQS ENSCTHEPGN LPNMLRDLRD
AFSRVKTFFQ MKDQLDNLLL KESLLEDEKG YLGCQALSEM IQFYLEEVMP QAENQDPDIK
AHVNSLGENL KTLRLRLRRC HRSLPCENKS KAVEQVKNAF NKLQEKGIYK AMSEFDIFIN
YIEAYMTMKI RN(GGGGSAA)ₙ₂PYAYWMRGG GGSDIQMTQS PSSLSASVGD RVTITCKTSQ
NIFENLAWYQ QKPGKAPKLL IYNASPLQAG VPSRESGSGS GTDETLTISS LQPEDFATYY
CHQYYSGYTF GPGTKLELKG GGGSGGGGSG GGGSQVQLVE SGGGVVQPGR SLRLSCAASG
FTESDYHMAW VRQAPGKGLE WVASITLDAT YTYYRDSVRG RFTISRDNSK NTLYLQMNSL
RAEDTAVYYC ARHRGFSVWL DYWGQGTLVT VSS; wherein n1 = 0, 1, 2, 3, 4,
or 5; and n2 = 0, 1, 2, 3, 4, or 5.
SEQ ID NO: 48-sarilumab anti-IL-6 receptor-IL10 homodimer 2
EVQLVESGGG LVQPGRSLRL SCAASRFTED DYAMHWVRQA PGKGLEWVSG ISWNSGRIGY
ADSVKGRFTI SRDNAENSLF LQMNGLRAED TALYYCAKGR DSFDIWGQGT MVTVSSASTK
GPSVFPLAPC SRSTSESTAA LGCLVKDYFP EPVTVSWNSG ALTSGVHTEP AVLQSSGLYS
LSSVVTVPSS SLGTKTYTCN VDHKPSNTKV DKRVESKYGP PCPPCPAPEF LGGPSVELEP
PKPKDTLMIS RTPEVTCVVV DVSQEDPEVQ FNWYVDGVEV HNAKTKPREE QFNSTYRVVS
VLTVLHQDWL NGKEYKCKVS NKGLPSSIEK TISKAKGQPR EPQVYTLPPS QEEMTKNQVS
LTCLVKGFYP SDIAVEWESN GQPENNYKTT PPVLDSDGSF FLYSRLTVDK SRWQEGNVES
CSVMHEALHN HYTQKSLSLS LGA(GGGGS)ₙ₁DIQMTQSPSS LSASVGDRVT ITCKTSQNIF
ENLAWYQQKP GKAPKLLIYN ASPLQAGVPS RESGSGSGTD FTLTISSLQP EDFATYYCHQ
YYSGYTFGPG TKLELKGGGG SGGGGsGGGG SQVQLVESGG GVVQPGRSLR LSCAASGFTF
SDYHMAWVRQ APGKGLEWVA SITLDATYTY YRDSVRGRET ISRDNSKNTL YLQMNSLRAE
DTAVYYCARH RGESVWLDYW GQGTLVTVSS (GGGGSAA)ₙ₂S PGQGTQSENS CTHEPGNLPN
MLRDLRDAFS RVKTFFQMKD QLDNLLLKES LLEDFKGYLG CQALSEMIQF YLEEVMPQAE
NQDPDIKAHV NSLGENLKTL RLRLRRCHRS LPCENKSKAV EQVKNAFNKL QEKGIYKAMS
EFDIFINYIE AYMTMKIRN; wherein n1 = 0, 1, 2, 3, 4, or 5; and n2 =
0, 1, 2, 3, 4, or 5.
SEQ ID NO: 49-sarilumab anti-IL-6 receptor-IL10 homodimer 3
EVQLVESGGG LVQPGRSLRL SCAASRFTED DYAMHWVRQA PGKGLEWVSG ISWNSGRIGY
ADSVKGRFTI SRDNAENSLF LQMNGLRAED TALYYCAKGR DSFDIWGQGT MVTVSSASTK
GPSVFPLAPC SRSTSESTAA LGCLVKDYFP EPVTVSWNSG ALTSGVHTEP AVLQSSGLYS
LSSVVTVPSS SLGTKTYTCN VDHKPSNTKV DKRVESKYGP PCPPCPAPEF LGGPSVELFP
PKPKDTLMIS RTPEVTCVVV DVSQEDPEVQ FNWYVDGVEV HNAKTKPREE QFNSTYRVVS
VLTVLHQDWL NGKEYKCKVS NKGLPSSIEK TISKAKGQPR EPQVYTLPPS QEEMTKNQVS
LTCLVKGFYP SDIAVEWESN GQPENNYKTT PPVLDSDGSF FLYSRLTVDK SRWQEGNVES
CSVMHEALHN HYTQKSLSLS LGA(GGGGSAA)ₙ₁QVQLVESG GGVVQPGRSL RLSCAASGFT
FSDYHMAWVR QAPGKGLEWV ASITLDATYT YYRDSVRGRF TISRDNSKNT LYLQMNSLRA
EDTAVYYCAR HRGFSVWLDY WGQGTLVTVS SGGGGSGGGG SGGGGSDIQM TQSPSSLSAS
VGDRVTITCK TSQNIFENLA WYQQKPGKAP KLLIYNASPL QAGVPSRESG SGSGTDETLT
ISSLQPEDFA TYYCHQYYSG YTFGPGTKLE LK(GGGGSAA)ₙ₂PYAYWMRGG GGSPGQGTQS
ENSCTHEPGN LPNMLRDLRD AFSRVKTFFQ MKDQLDNLLL KESLLEDEKG YLGCQALSEM
IQFYLEEVMP QAENQDPDIK AHVNSLGENL KTLRLRLRRC HRSLPCENKS KAVEQVKNAF
NKLQEKGIYK AMSEFDIFIN YIEAYMTMKI RN; wherein n1 = 0, 1, 2, 3, 4,
or 5; n2 = 1, 2, 3, 4, or 5.
SEQ ID NO: 50-sarilumab anti-IL-6 receptor-IL10 homodimer 4
EVQLVESGGG LVQPGRSLRL SCAASRFTED DYAMHWVRQA PGKGLEWVSG ISWNSGRIGY
ADSVKGRFTI SRDNAENSLF LQMNGLRAED TALYYCAKGR DSFDIWGQGT MVTVSSASTK
GPSVFPLAPC SRSTSESTAA LGCLVKDYFP EPVTVSWNSG ALTSGVHTEP AVLQSSGLYS
LSSVVTVPSS SLGTKTYTCN VDHKPSNTKV DKRVESKYGP PCPPCPAPEF LGGPSVELEP
PKPKDTLMIS RTPEVTCVVV DVSQEDPEVQ FNWYVDGVEV HNAKTKPREE QFNSTYRVVS
VLTVLHQDWL NGKEYKCKVS NKGLPSSIEK TISKAKGQPR EPQVYTLPPS QEEMTKNQVS
LTCLVKGFYP SDIAVEWESN GQPENNYKTT PPVLDSDGSF FLYSRLTVDK SRWQEGNVES
CSVMHEALHN HYTQKSLSLS LGA(GGGGSAA)ₙ₁QVQLVESG GGVVQPGRSL RLSCAASGFT
FSDYHMAWVR QAPGKGLEWV ASITLDATYT YYRDSVRGRF TISRDNSKNT LYLQMNSLRA
EDTAVYYCAR HRGFSVWLDY WGQGTLVTVS SGGGGSGGGG SGGGGSDIQM TQSPSSLSAS
VGDRVTITCK TSQNIFENLA WYQQKPGKAP KLLIYNASPL QAGVPSRESG SGSGTDETLT
ISSLQPEDFA TYYCHQYYSG YTFGPGTKLE LK(GGGGSAA)ₙ₂PYAYWMRGG GGSPGQGTQS
ENSCTHEPGN LPNMLRDLRD AFSRVKTFFQ MKDQLDNLLL KESLLEDEKG YLGCQALSEM
IQFYLEEVMP QAENQDPDIK AHVNSLGENL KTLRLRLRRC HRSLPCENKS KAVEQVKNAF
NKLQEKGIYK AMSEFDIFIN YIEAYMTMKI RN; wherein n1 = 0, 1, 2, 3, 4,
or 5; n2 = 1, 2, 3, 4, or 5.
SEQ ID NO: 51-anti-IFNAR-1 9D4, VH
EVQLVQSGAE VKKPGESLKI SCKGSGYIFT NYWIAWVRQM PGKGLESMGI IYPGDSDIRY
SPSFQGQVTI SADKSITTAY LQWSSLKASD TAMYYCARHD IEGEDYWGRG TLVTVSS
SEQ ID NO: 52-anti-IFNAR-1 9D4, VL
EIVLTQSPGT LSLSPGERAT LSCRASQSVS SSFFAWYQQK PGQAPRLLIY GASSRATGIP
DRLSGSGSGT DETLTITRLE PEDFAVYYCQ QYDSSAITFG QGTRLEIK
SEQ ID NO: 53-anti-IFNAR-1 9D4, Light chain
EIVLTQSPGT LSLSPGERAT LSCRASQSVS SSFFAWYQQK PGQAPRLLIY GASSRATGIP
DRLSGSGSGT DETLTITRLE PEDFAVYYCQ QYDSSAITFG QGTRLEIKRT VAAPSVEIFP
PSDEQLKSGT ASVVCLLNNF YPREAKVQWK VDNALQSGNS QESVTEQDSK DSTYSLSSTL
TLSKADYEKH KVYACEVTHQ GLSSPVTKSF NRGEC
SEQ ID NO: 54-anti-IFNAR-1 9D4-IL10 knob
EVQLVQSGAE VKKPGESLKI SCKGSGYIFT NYWIAWVRQM PGKGLESMGI IYPGDSDIRY
SPSFQGQVTI SADKSITTAY LQWSSLKASD TAMYYCARHD IEGEDYWGRG TLVTVSSAST
KGPSVFPLAP CSRSTSESTA ALGCLVKDYF PEPVTVSWNS GALTSGVHTE PAVLQSSGLY
SLSSVVTVPS SSLGTKTYTC NVDHKPSNTK VDKRVESKYG PPCPPCPAPE FLGGPSVELF
PPKPKDTLYI TREPEVTCVV VDVSQEDPEV QFNWYVDGVE VHNAKTKPRE EQENSTYRVV
SVLTVLHQDW LNGKEYKCKV SNKGLPSSIE KTISKAKGQP REPQVYTLPP CQEEMTKNQV
SLWCLVKGFY PSDIAVEWES NGQPENNYKT TPPVLDSDGS FFLYSRLTVD KSRWQEGNVE
SCSVMHEALH NHYTQKSLSL SLGA(GGGGS)ₙ₁SPGQGTQSE NSCTHEPGNL PNMLRDLRDA
FSRVKTFFQM KDQLDNLLLK ESLLEDEKGY LGCQALSEMI QFYLEEVMPQ AENQDPDIKA
HVNSLGENLK TLRLRLRRCH RSLPCENKSK AVEQVKNAFN KLQEKGIYKA MSEFDIFINY
IEAYMTMKIR N[(GGGGSAA)ₙ₂SPGQGTQSE NSCTHEPGNL PNMLRDLRDA FSRVKTFFQM
KDQLDNLLLK ESLLEDFKGY LGCQALSEMI QFYLEEVMPQ AENQDPDIKA HVNSLGENLK
TLRLRLRRCH RSLPCENKSK AVEQVKNAFN KLQEKGIYKA MSEFDIFINY IEAYMTMKIR
N]n3; wherein n1 = 0, 1, 2, or 3; n2 = 1, 2, 3, 4, or 5; and n3 =
0 or 1.
SEQ ID NO: 55-anti-IFNAR-1 9D4-IL10-scFv1 hole
EVQLVQSGAE VKKPGESLKI SCKGSGYIFT NYWIAWVRQM PGKGLESMGI IYPGDSDIRY
SPSFQGQVTI SADKSITTAY LQWSSLKASD TAMYYCARHD IEGEDYWGRG TLVTVSSAST
KGPSVFPLAP CSRSTSESTA ALGCLVKDYF PEPVTVSWNS GALTSGVHTE PAVLQSSGLY
SLSSVVTVPS SSLGTKTYTC NVDHKPSNTK VDKRVESKYG PPCPPCPAPE FLGGPSVELE
PPKPKDTLYI TREPEVTCVV VDVSQEDPEV QFNWYVDGVE VHNAKTKPRE EQFNSTYRVV
SVLTVLHQDW LNGKEYKCKV SNKGLPSSIE KTISKAKGQP REPQVCTLPP SQEEMTKNQV
SLSCAVKGFY PSDIAVEWES NGQPENNYKT TPPVLDSDGS FFLVSRLTVD KSRWQEGNVE
SCSVMHEALH NHYTQKSLSL SLGA(GGGGSAA)ₙ₁DIQMTQS PSSLSASVGD RVTITCKTSQ
NIFENLAWYQ QKPGKAPKLL IYNASPLQAG VPSRESGSGS GTDETLTISS LQPEDFATYY
CHQYYSGYTF GPGTKLELKG GGGSGGGGSG GGGSQVQLVE SGGGVVQPGR SLRLSCAASG
FTESDYHMAW VRQAPGKGLE WVASITLDAT YTYYRDSVRG RFTISRDNSK NTLYLQMNSL
RAEDTAVYYC ARHRGFSVWL DYWGQGTLVT VSS[(GGGGSAA)ₙ₂(PYAYWMR)ₙ₃(GGGGS)ₙ₄
DIQMTQSPSS LSASVGDRVT ITCKTSQNIF ENLAWYQQKP GKAPKLLIYN ASPLQAGVPS
RFSGSGSGTD FTLTISSLQP EDFATYYCHQ YYSGYTFGPG TKLELKGGGG SGGGGSGGGG
SQVQLVESGG GVVQPGRSLR LSCAASGFTF SDYHMAWVRQ APGKGLEWVA SITLDATYTY
YRDSVRGRFT ISRDNSKNTL YLQMNSLRAE DTAVYYCARH RGFSVWLDYW GQGTLVTVSS]ₙ₅;
wherein n1 = 1, 2, 3, or 4; n2 = 1, 2, 3, 4, or 5; n3 = 0 or 1;
n4 = 0, 1, or 2; and n5 = 0, or 1.
SEQ ID NO: 56-anti-IFNAR-1 9D4-IL10-scFv2 hole
EVQLVQSGAE VKKPGESLKI SCKGSGYIFT NYWIAWVRQM PGKGLESMGI IYPGDSDIRY
SPSFQGQVTI SADKSITTAY LQWSSLKASD TAMYYCARHD IEGFDYWGRG TLVTVSSAST
KGPSVFPLAP CSRSTSESTA ALGCLVKDYF PEPVTVSWNS GALTSGVHTE PAVLQSSGLY
SLSSVVTVPS SSLGTKTYTC NVDHKPSNTK VDKRVESKYG PPCPPCPAPE FLGGPSVELE
PPKPKDTLYI TREPEVTCVV VDVSQEDPEV QFNWYVDGVE VHNAKTKPRE EQFNSTYRVV
SVLTVLHQDW LNGKEYKCKV SNKGLPSSIE KTISKAKGQP REPQVCTLPP SQEEMTKNQV
SLSCAVKGFY PSDIAVEWES NGQPENNYKT TPPVLDSDGS FFLVSRLTVD KSRWQEGNVE
SCSVMHEALH NHYTQKSLSL SLGA(GGGGSAA)ₙ₁QVQLVES GGGVVQPGRS LRLSCAASGE
TFSDYHMAWV RQAPGKGLEW VASITLDATY TYYRDSVRGR FTISRDNSKN TLYLQMNSLR
AEDTAVYYCA RHRGESVWLD YWGQGTLVTV SSGGGGSGGG GSGGGGSDIQ MTQSPSSLSA
SVGDRVTITC KTSQNIFENL AWYQQKPGKA PKLLIYNASP LQAGVPSRES GSGSGTDETL
TISSLQPEDF ATYYCHQYYS GYTFGPGTKL ELK[(GGGGSAA)ₙ₂(PYAYWMR)ₙ₃(GGGGS)ₙ₄
QVQLVESGGG VVQPGRSLRL SCAASGFTFS DYHMAWVRQA PGKGLEWVAS ITLDATYTYY
RDSVRGRFTI SRDNSKNTLY LQMNSLRAED TAVYYCARHR GFSVWLDYWG QGTLVTVSSG
GGGSGGGGSG GGGSDIQMTQ SPSSLSASVG DRVTITCKTS QNIFENLAWY QQKPGKAPKL
LIYNASPLQA GVPSRESGSG SGTDETLTIS SLQPEDFATY YCHQYYSGYT FGPGTKLELK]ₙ₅;
wherein n1 = 1, 2, 3, or 4; n2 = 1, 2, 3, 4, or 5; n3 = 0 or 1;
n4 = 0, 1, or 2; and n5 = 0, or 1.
SEQ ID NO: 57-anti-IFNAR-1 9D4-IL10 homodimer 1
EVQLVQSGAE VKKPGESLKI SCKGSGYIFT NYWIAWVRQM PGKGLESMGI IYPGDSDIRY
SPSFQGQVTI SADKSITTAY LQWSSLKASD TAMYYCARHD IEGEDYWGRG TLVTVSSAST
KGPSVFPLAP CSRSTSESTA ALGCLVKDYF PEPVTVSWNS GALTSGVHTF PAVLQSSGLY
SLSSVVTVPS SSLGTKTYTC NVDHKPSNTK VDKRVESKYG PPCPPCPAPE FLGGPSVELE
PPKPKDTLMI SRTPEVTCVV VDVSQEDPEV QFNWYVDGVE VHNAKTKPRE EQFNSTYRVV
SVLTVLHQDW LNGKEYKCKV SNKGLPSSIE KTISKAKGQP REPQVYTLPP SQEEMTKNQV
SLTCLVKGFY PSDIAVEWES NGQPENNYKT TPPVLDSDGS FFLYSRLTVD KSRWQEGNVE
SCSVMHEALH NHYTQKSLSL SLGA(GGGGSAA)ₙ₁SPGQGTQ SENSCTHEPG NLPNMLRDLR
DAFSRVKTFF QMKDQLDNLL LKESLLEDFK GYLGCQALSE MIQFYLEEVM PQAENQDPDI
KAHVNSLGEN LKTLRLRLRR CHRSLPCENK SKAVEQVKNA FNKLQEKGIY KAMSEFDIFI
NYIEAYMTMK IRN(GGGGSAA)ₙ₂PYAYWMRG GGGSDIQMTQ SPSSLSASVG DRVTITCKTS
QNIFENLAWY QQKPGKAPKL LIYNASPLQA GVPSRESGSG SGTDETLTIS SLQPEDFATY
YCHQYYSGYT FGPGTKLELK GGGGSGGGGS GGGGSQVQLV ESGGGVVQPG RSLRLSCAAS
GFTFSDYHMA WVRQAPGKGL EWVASITLDA TYTYYRDSVR GRFTISRDNS KNTLYLQMNS
LRAEDTAVYY CARHRGESVW LDYWGQGTLV TVSS; wherein n1 = 0, 1, 2, 3, 4,
or 5; and n2 = 0, 1, 2, 3, 4, or 5.
SEQ ID NO: 58-Anti-IFNAR-1 9D4-IL10 Homodimer 2
EVQLVQSGAE VKKPGESLKI SCKGSGYIFT NYWIAWVRQM PGKGLESMGI IYPGDSDIRY
SPSFQGQVTI SADKSITTAY LQWSSLKASD TAMYYCARHD IEGEDYWGRG TLVTVSSAST
KGPSVEPLAP CSRSTSESTA ALGCLVKDYF PEPVTVSWNS GALTSGVHTF PAVLQSSGLY
SLSSVVTVPS SSLGTKTYTC NVDHKPSNTK VDKRVESKYG PPCPPCPAPE FLGGPSVFLF
PPKPKDTLMI SRTPEVTCVV VDVSQEDPEV QFNWYVDGVE VHNAKTKPRE EQENSTYRVV
SVLTVLHQDW LNGKEYKCKV SNKGLPSSIE KTISKAKGQP REPQVYTLPP SQEEMTKNQV
SLTCLVKGFY PSDIAVEWES NGQPENNYKT TPPVLDSDGS FFLYSRLTVD KSRWQEGNVE
SCSVMHEALH NHYTQKSLSL SLGA(GGGGS)ₙ₁DIQMTQSPS SLSASVGDRV TITCKTSQNI
FENLAWYQQK PGKAPKLLIY NASPLQAGVP SRESGSGSGT DFTLTISSLQ PEDFATYYCH
QYYSGYTFGP GTKLELKGGG GSGGGGSGGG GSQVQLVESG GGVVQPGRSL RLSCAASGET
FSDYHMAWVR QAPGKGLEWV ASITLDATYT YYRDSVRGRF TISRDNSKNT LYLQMNSLRA
EDTAVYYCAR HRGFSVWLDY WGQGTLVTVS S(GGGGSAA)ₙ₂SPGQGTQSEN SCTHEPGNLP
NMLRDLRDAF SRVKTFFQMK DQLDNLLLKE SLLEDEKGYL GCQALSEMIQ FYLEEVMPQA
ENQDPDIKAH VNSLGENLKT LRLRLRRCHR SLPCENKSKA VEQVKNAFNK LQEKGIYKAM
SEFDIFINYI EAYMTMKIRN; wherein n1 = 0, 1, 2, 3, 4, or 5; and n2 =
0, 1, 2, 3, 4, or 5.
SEQ ID NO: 59-anti-IFNAR-1 9D4-IL10 homodimer 3
EVQLVQSGAE VKKPGESLKI SCKGSGYIFT NYWIAWVRQM PGKGLESMGI IYPGDSDIRY
SPSFQGQVTI SADKSITTAY LQWSSLKASD TAMYYCARHD IEGEDYWGRG TLVTVSSAST
KGPSVFPLAP CSRSTSESTA ALGCLVKDYF PEPVTVSWNS GALTSGVHTF PAVLQSSGLY
SLSSVVTVPS SSLGTKTYTC NVDHKPSNTK VDKRVESKYG PPCPPCPAPE FLGGPSVELE
PPKPKDTLMI SRTPEVTCVV VDVSQEDPEV QFNWYVDGVE VHNAKTKPRE EQENSTYRVV
SVLTVLHQDW LNGKEYKCKV SNKGLPSSIE KTISKAKGQP REPQVYTLPP SQEEMTKNQV
SLTCLVKGFY PSDIAVEWES NGQPENNYKT TPPVLDSDGS FFLYSRLTVD KSRWQEGNVE
SCSVMHEALH NHYTQKSLSL SLGA(GGGGSAA)ₙ₁ SPGQGT QSENSCTHEP GNLPNMLRDL
RDAFSRVKTF FQMKDQLDNL LLKESLLEDF KGYLGCQALS EMIQFYLEEV MPQAENQDPD
IKAHVNSLGE NLKTLRLRLR RCHRSLPCEN KSKAVEQVKN AFNKLQEKGI YKAMSEFDIE
INYIEAYMTM KIRN(GGGGSAA)ₙ₂PYAYWMR GGGGSQVQLV ESGGGVVQPG RSLRLSCAAS
GFTFSDYHMA WVRQAPGKGL EWVASITLDA TYTYYRDSVR GRFTISRDNS KNTLYLQMNS
LRAEDTAVYY CARHRGESVW LDYWGQGTLV TVSSGGGGSG GGGSGGGGSD IQMTQSPSSL
SASVGDRVTI TCKTSQNIFE NLAWYQQKPG KAPKLLIYNA SPLQAGVPSR FSGSGSGTDE
TLTISSLQPE DEATYYCHQY YSGYTEGPGT KLELK; wherein n1 = 0, 1, 2, 3,
4, or 5; n2 = 1, 2, 3, 4, or 5.
SEQ ID NO: 60-anti-IFNAR-1 9D4-IL10 homodimer 4
EVQLVQSGAE VKKPGESLKI SCKGSGYIFT NYWIAWVRQM PGKGLESMGI IYPGDSDIRY
SPSFQGQVTI SADKSITTAY LQWSSLKASD TAMYYCARHD IEGEDYWGRG TLVTVSSAST
KGPSVFPLAP CSRSTSESTA ALGCLVKDYF PEPVTVSWNS GALTSGVHTF PAVLQSSGLY
SLSSVVTVPS SSLGTKTYTC NVDHKPSNTK VDKRVESKYG PPCPPCPAPE FLGGPSVELF
PPKPKDTLMI SRTPEVTCVV VDVSQEDPEV QFNWYVDGVE VHNAKTKPRE EQFNSTYRVV
SVLTVLHQDW LNGKEYKCKV SNKGLPSSIE KTISKAKGQP REPQVYTLPP SQEEMTKNQV
SLTCLVKGFY PSDIAVEWES NGQPENNYKT TPPVLDSDGS FFLYSRLTVD KSRWQEGNVE
SCSVMHEALH NHYTQKSLSL SLGA(GGGGSAA)ₙ₁QVQLVES GGGVVQPGRS LRLSCAASGF
TFSDYHMAWV RQAPGKGLEW VASITLDATY TYYRDSVRGR FTISRDNSKN TLYLQMNSLR
AEDTAVYYCA RHRGESVWLD YWGQGTLVTV SSGGGGSGGG GSGGGGSDIQ MTQSPSSLSA
SVGDRVTITC KTSQNIFENL AWYQQKPGKA PKLLIYNASP LQAGVPSRES GSGSGTDETL
TISSLQPEDF ATYYCHQYYS GYTFGPGTKL ELK(GGGGSAA)ₙ₂SPGQGTQS ENSCTHEPGN
LPNMLRDLRD AFSRVKTFFQ MKDQLDNLLL KESLLEDEKG YLGCQALSEM IQFYLEEVMP
QAENQDPDIK AHVNSLGENL KTLRLRLRRC HRSLPCENKS KAVEQVKNAF NKLQEKGIYK
AMSEFDIFIN YIEAYMTMKI RN; wherein n1 = 0, 1, 2, 3, 4, or 5; n2 = 1,
2, 3, 4, or 5.
SEQ ID NO: 61-CTLA-4 ECD mutant belatacept
MHVAQPAVVL ASSRGIASFV CEYASPGKYT EVRVTVLRQA DSQVTEVCAA TYMMGNELTE
LDDSICTGTS SGNQVNLTIQ GLRAMDTGLY ICKVELMYPP PYYEGIGNGT QIYVIDPEPC
SEQ ID NO: 62-M3 peptide
RGFFRGG
SEQ ID NO: 63-CTLA-4 ECD mutant
MHVAQPAVVL ASSRGIASFV CEYASPGKYT EVRVTVLRQA DSQVTEVCAA TYMMGNELTE
LDDSICTGTS SGNQVNLTIQ GLRAMDTGLY ICKVELMYPP PYYEGIGNGT QIYVIDPEPC
PDSDQEPKSS AESKYGPPCP PCPAPEFLGG PSVELFPPKP KDTLYITREP EVTCVVVDVS
QEDPEVQFNW YVDGVEVHNA KTKPREEQEN STYRVVSVLT VLHQDWLNGK EYKCKVSNKG
LPSSIEKTIS KAKGQPREPQ VYTLPPCQEE MTKNQVSLWC LVKGFYPSDI AVEWESNGQP
ENNYKTTPPV LDSDGSFFLY SRLTVDKSRW QEGNVESCSV MHEALHNHYT
QKSLSLSLGA (GGGGS)ₙ₁SPG QGTQSENSCT HFPGNLPNML RDLRDAFSRV KTFFQMKDQL
DNLLLKESLL EDFKGYLGCQ ALSEMIQFYL EEVMPQAENQ DPDIKAHVNS LGENLKTLRL
RLRRCHRSLP CENKSKAVEQ VKNAFNKLQE KGIYKAMSEF DIFINYIEAY
MTMKIRN[(GGGGSAA)ₙ₂SPG QGTQSENSCT HFPGNLPNML RDLRDAFSRV KTFFQMKDQL
DNLLLKESLL EDFKGYLGCQ ALSEMIQFYL EEVMPQAENQ DPDIKAHVNS LGENLKTLRL
RLRRCHRSLP CENKSKAVEQ VKNAFNKLQE KGIYKAMSEF DIFINYIEAY MTMKIRN]ₙ₃;
wherein n1 = 0, 1, 2, or 3; n2 = 1, 2, 3, 4, or 5; and n3 = 0 or
1.
SEQ ID NO: 64-CTLA-4 ECD mutant belatacept-IL10-scFv1 hole
MHVAQPAVVL ASSRGIASFV CEYASPGKYT EVRVTVLRQA DSQVTEVCAA TYMMGNELTE
LDDSICTGTS SGNQVNLTIQ GLRAMDTGLY ICKVELMYPP PYYEGIGNGT QIYVIDPEPC
PDSDQEPKSS AESKYGPPCP PCPAPEELGG PSVELFPPKP KDTLYITREP EVTCVVVDVS
QEDPEVQFNW YVDGVEVHNA KTKPREEQEN STYRVVSVLT VLHQDWLNGK EYKCKVSNKG
LPSSIEKTIS KAKGQPREPQ VCTLPPSQEE MTKNQVSLSC AVKGFYPSDI AVEWESNGQP
ENNYKTTPPV LDSDGSFFLV SRLTVDKSRW QEGNVESCSV MHEALHNHYT
QKSLSLSLGA (GGGGSAA)ₙ₁D IQMTQSPSSL SASVGDRVTI TCKTSQNIFE NLAWYQQKPG
KAPKLLIYNA SPLQAGVPSR FSGSGSGTDF TLTISSLQPE DFATYYCHQY YSGYTFGPGT
KLELKGGGGS GGGGSGGGGS QVQLVESGGG VVQPGRSLRL SCAASGFTES DYHMAWVRQA
PGKGLEWVAS ITLDATYTYY RDSVRGRFTI SRDNSKNTLY LQMNSLRAED TAVYYCARHR
GFSVWLDYWG QGTLVTVSS[(GGGGSAA)ₙ₂(PYAYWMR)ₙ₃(GGGGS)ₙ₄DIQM TQSPSSLSAS
VGDRVTITCK TSQNIFENLA WYQQKPGKAP KLLIYNASPL QAGVPSRESG SGSGTDFTLT
ISSLQPEDFA TYYCHQYYSG YTFGPGTKLE LKGGGGSGGG GSGGGGSQVQ LVESGGGVVQ
PGRSLRLSCA ASGFTFSDYH MAWVRQAPGK GLEWVASITL DATYTYYRDS VRGRFTISRD
NSKNTLYLQM NSLRAEDTAV YYCARHRGES VWLDYWGQGT LVTVSS]ₙ₅; wherein
n1 = 1, 2, 3, or 4; n2 = 1, 2, 3, 4, or 5; n3 = 0 or 1; n4 = 0,
1, or 2; and n5 = 0, or 1.
SEQ ID NO: 65-CTLA-4 ECD mutant belatacept -IL10-scFv2 hole
MHVAQPAVVL ASSRGIASFV CEYASPGKYT EVRVTVLRQA DSQVTEVCAA TYMMGNELTE
LDDSICTGTS SGNQVNLTIQ GLRAMDTGLY ICKVELMYPP PYYEGIGNGT QIYVIDPEPC
PDSDQEPKSS AESKYGPPCP PCPAPEFLGG PSVELFPPKP KDTLYITREP EVTCVVVDVS
QEDPEVQFNW YVDGVEVHNA KTKPREEQEN STYRVVSVLT VLHQDWLNGK EYKCKVSNKG
LPSSIEKTIS KAKGQPREPQ VCTLPPSQEE MTKNQVSLSC AVKGFYPSDI AVEWESNGQP
ENNYKTTPPV LDSDGSFFLV SRLTVDKSRW QEGNVESCSV MHEALHNHYT
QKSLSLSLGA (GGGGSAA)ₙ₁Q VQLVESGGGV VQPGRSLRLS CAASGFTESD YHMAWVRQAP
GKGLEWVASI TLDATYTYYR DSVRGRFTIS RDNSKNTLYL QMNSLRAEDT AVYYCARHRG
FSVWLDYWGQ GTLVTVSSGG GGSGGGGSGG GGSDIQMTQS PSSLSASVGD RVTITCKTSQ
NIFENLAWYQ QKPGKAPKLL IYNASPLQAG VPSRESGSGS GTDETLTISS LQPEDFATYY
CHQYYSGYTF GPGTKLELK[(GGGGSAA)ₙ₂(PYAYWMR)ₙ₃(GGGGS)ₙ₄QVQ LVESGGGVVQ
PGRSLRLSCA ASGFTESDYH MAWVRQAPGK GLEWVASITL DATYTYYRDS VRGRFTISRD
NSKNTLYLQM NSLRAEDTAV YYCARHRGFS VWLDYWGQGT LVTVSSGGGG SGGGGSGGGG
SDIQMTQSPS SLSASVGDRV TITCKTSQNI FENLAWYQQK PGKAPKLLIY NASPLQAGVP
SRFSGSGSGT DFTLTISSLQ PEDFATYYCH QYYSGYTFGP GTKLELK]ₙ₅; wherein
n1 = 1, 2, 3, or 4; n2 = 1, 2, 3, 4, or 5; n3 = 0 or 1; n4 = 0,
1, or 2; and n5 = 0, or 1.
SEQ ID NO: 66-CTLA-4 ECD mutant belatacept -IL10-homodimer 1
MHVAQPAVVL ASSRGIASFV CEYASPGKYT EVRVTVLRQA DSQVTEVCAA TYMMGNELTE
LDDSICTGTS SGNQVNLTIQ GLRAMDTGLY ICKVELMYPP PYYEGIGNGT QIYVIDPEPC
PDSDQEPKSS AESKYGPPCP PCPAPEFLGG PSVELFPPKP KDTLMISRTP EVTCVVVDVS
QEDPEVQFNW YVDGVEVHNA KTKPREEQFN STYRVVSVLT VLHQDWLNGK EYKCKVSNKG
LPSSIEKTIS KAKGQPREPQ VYTLPPSQEE MTKNQVSLTC LVKGFYPSDI AVEWESNGQP
ENNYKTTPPV LDSDGSFFLY SRLTVDKSRW QEGNVESCSV MHEALHNHYT QKSLSLSLGA
(GGGGSAA)ₙ₁ SPGQGTQSEN SCTHEPGNLP NMLRDLRDAF SRVKTFFQMK DQLDNLLLKE
SLLEDEKGYL GCQALSEMIQ FYLEEVMPQA ENQDPDIKAH VNSLGENLKT LRLRLRRCHR
SLPCENKSKA VEQVKNAFNK LQEKGIYKAM SEFDIFINYI EAYMTMKIRN (GGGGSAA)ₙ₂P
YAYWMRGGGG SDIQMTQSPS SLSASVGDRV TITCKTSQNI FENLAWYQQK PGKAPKLLIY
NASPLQAGVP SRESGSGSGT DFTLTISSLQ PEDFATYYCH QYYSGYTEGP GTKLELKGGG
GSGGGGSGGG GSQVQLVESG GGVVQPGRSL RLSCAASGFT FSDYHMAWVR QAPGKGLEWV
ASITLDATYT YYRDSVRGRE TISRDNSKNT LYLQMNSLRA EDTAVYYCAR HRGESVWLDY
WGQGTLVTVS S; wherein n1 = 0, 1, 2, 3, 4, or 5; and n2 = 0, 1, 2,
3, 4, or 5.
SEQ ID NO: 67-CTLA-4 ECD mutant belatacept-IL10-homodimer 2
MHVAQPAVVL ASSRGIASFV CEYASPGKYT EVRVTVLRQA DSQVTEVCAA TYMMGNELTF
LDDSICTGTS SGNQVNLTIQ GLRAMDTGLY ICKVELMYPP PYYEGIGNGT QIYVIDPEPC
PDSDQEPKSS AESKYGPPCP PCPAPEFLGG PSVFLFPPKP KDTLMISRTP EVTCVVVDVS
QEDPEVQENW YVDGVEVHNA KTKPREEQEN STYRVVSVLT VLHQDWLNGK EYKCKVSNKG
LPSSIEKTIS KAKGQPREPQ VYTLPPSQEE MTKNQVSLTC LVKGFYPSDI AVEWESNGQP
ENNYKTTPPV LDSDGSFFLY SRLTVDKSRW QEGNVESCSV MHEALHNHYT QKSLSLSLGA
(GGGGS)ₙ₁DI QMTQSPSSLS ASVGDRVTIT CKTSQNIFEN LAWYQQKPGK APKLLIYNAS
PLQAGVPSRF SGSGSGTDFT LTISSLQPED FATYYCHQYY SGYTFGPGTK LELKGGGGSG
GGGSGGGGSQ VQLVESGGGV VQPGRSLRLS CAASGFTESD YHMAWVRQAP GKGLEWVASI
TLDATYTYYR DSVRGRETIS RDNSKNTLYL QMNSLRAEDT AVYYCARHRG FSVWLDYWGQ
GTLVTVSS(GGGGSAA)ₙ₂SP GQGTQSENSC THEPGNLPNM LRDLRDAFSR VKTFFQMKDQ
LDNLLLKESL LEDFKGYLGC QALSEMIQFY LEEVMPQAEN QDPDIKAHVN SLGENLKTLR
LRLRRCHRSL PCENKSKAVE QVKNAFNKLQ EKGIYKAMSE FDIFINYIEA YMTMKIRN;
wherein n1 = 0, 1, 2, 3, 4, or 5; and n2 = 0, 1, 2, 3, 4, or 5.
SEQ ID NO: 68-CTLA-4 ECD mutant belatacept-IL10-homodimer 3
MHVAQPAVVL ASSRGIASFV CEYASPGKYT EVRVTVLRQA DSQVTEVCAA TYMMGNELTE
LDDSICTGTS SGNQVNLTIQ GLRAMDTGLY ICKVELMYPP PYYEGIGNGT QIYVIDPEPC
PDSDQEPKSS AESKYGPPCP PCPAPEFLGG PSVFLFPPKP KDTLMISRTP EVTCVVVDVS
QEDPEVQFNW YVDGVEVHNA KTKPREEQFN STYRVVSVLT VLHQDWLNGK EYKCKVSNKG
LPSSIEKTIS KAKGQPREPQ VYTLPPSQEE MTKNQVSLTC LVKGFYPSDI AVEWESNGQP
ENNYKTTPPV LDSDGSFFLY SRLTVDKSRW QEGNVESCSV MHEALHNHYT
QKSLSLSLGA (GGGGSAA)ₙ₁S PGQGTQSENS CTHEPGNLPN MLRDLRDAFS RVKTFFQMKD
QLDNLLLKES LLEDFKGYLG CQALSEMIQF YLEEVMPQAE NQDPDIKAHV NSLGENLKTL
RLRLRRCHRS LPCENKSKAV EQVKNAFNKL QEKGIYKAMS EFDIFINYIE
AYMTMKIRN (GGGGSAA)ₙ₂PY AYWMRGGGGS QVQLVESGGG VVQPGRSLRL SCAASGFTES
DYHMAWVRQA PGKGLEWVAS ITLDATYTYY RDSVRGRFTI SRDNSKNTLY LQMNSLRAED
TAVYYCARHR GFSVWLDYWG QGTLVTVSSG GGGSGGGGSG GGGSDIQMTQ SPSSLSASVG
DRVTITCKTS QNIFENLAWY QQKPGKAPKL LIYNASPLQA GVPSRESGSG SGTDETLTIS
SLQPEDFATY YCHQYYSGYT FGPGTKLELK; wherein n1 = 0, 1, 2, 3, 4, or
5; n2 = 1, 2, 3, 4, or 5.
SEQ ID NO: 69-CTLA-4 ECD mutant belatacept-IL10-homodimer 4
MHVAQPAVVL ASSRGIASFV CEYASPGKYT EVRVTVLRQA DSQVTEVCAA TYMMGNELTE
LDDSICTGTS SGNQVNLTIQ GLRAMDTGLY ICKVELMYPP PYYEGIGNGT QIYVIDPEPC
PDSDQEPKSS AESKYGPPCP PCPAPEFLGG PSVELFPPKP KDTLMISRTP EVTCVVVDVS
QEDPEVQFNW YVDGVEVHNA KTKPREEQFN STYRVVSVLT VLHQDWLNGK EYKCKVSNKG
LPSSIEKTIS KAKGQPREPQ VYTLPPSQEE MTKNQVSLTC LVKGFYPSDI AVEWESNGQP
ENNYKTTPPV LDSDGSFFLY SRLTVDKSRW QEGNVFSCSV MHEALHNHYT QKSLSLSLGA
(GGGGSAA)ₙ₁ QVQLVESGGG VVQPGRSLRL SCAASGFTES DYHMAWVRQA PGKGLEWVAS
ITLDATYTYY RDSVRGRFTI SRDNSKNTLY LQMNSLRAED TAVYYCARHR GESVWLDYWG
QGTLVTVSSG GGGSGGGGSG GGGSDIQMTQ SPSSLSASVG DRVTITCKTS QNIFENLAWY
QQKPGKAPKL LIYNASPLQA GVPSRESGSG SGTDETLTIS SLQPEDFATY YCHQYYSGYT
FGPGTKLELK (GGGGSAA)ₙ₂ SPGQGTQSEN SCTHEPGNLP NMLRDLRDAF SRVKTFFQMK
DQLDNLLLKE SLLEDEKGYL GCQALSEMIQ FYLEEVMPQA ENQDPDIKAH VNSLGENLKT
LRLRLRRCHR SLPCENKSKA VEQVKNAFNK LQEKGIYKAM SEFDIFINYI EAYMTMKIRN;
wherein n1 = 0, 1, 2, 3, 4, or 5; n2 = 1, 2, 3, 4, or 5.
SEQ ID NO: 70-TNFR-2 ECD-IL10-knob
LPAQVAFTPY APEPGSTCRL REYYDQTAQM CCSKCSPGQH AKVFCTKTSD TVCDSCEDST
YTQLWNWVPE CLSCGSRCSS DQVETQACTR EQNRICTCRP GWYCALSKQE GCRLCAPLRK
CRPGFGVARP GTETSDVVCK PCAPGTESNT TSSTDICRPH QICNVVAIPG NASMDAVCTS
TSPTRSMAPG AVHLPQPVST RSQHTQPTPE PSTAPSTSFL LPMGPSPPAE GSTGDQEPKS
SAESKYGPPC PPCPAPEFLG GPSVFLFPPK PKDTLYITRE PEVTCVVVDV SQEDPEVQEN
WYVDGVEVHN AKTKPREEQF NSTYRVVSVL TVLHQDWING KEYKCKVSNK GLPSSIEKTI
SKAKGQPREP QVYTLPPCQE EMTKNQVSLW CLVKGFYPSD IAVEWESNGQ PENNYKTTPP
VLDSDGSFFL YSRLTVDKSR WQEGNVESCS VMHEALHNHY TQKSLSLSLG A(GGGGS)ₙ₁S
PGQGTQSENS CTHEPGNLPN MLRDLRDAFS RVKTFFQMKD QLDNLLLKES LLEDFKGYLG
CQALSEMIQF YLEEVMPQAE NQDPDIKAHV NSLGENLKTL RLRLRRCHRS LPCENKSKAV
EQVKNAFNKL QEKGIYKAMS EFDIFINYIE AYMTMKIRN[(GGGGSAA)ₙ₂S PGQGTQSENS
CTHEPGNLPN MLRDLRDAFS RVKTFFQMKD QLDNLLLKES LLEDFKGYLG CQALSEMIQF
YLEEVMPQAE NQDPDIKAHV NSLGENLKTL RLRLRRCHRS LPCENKSKAV EQVKNAENKL
QEKGIYKAMS EFDIFINYIE AYMTMKIRN]ₙ₃; wherein n1 = 0, 1, 2, or 3;
n2 = 1, 2, 3, 4, or 5; and n3 = 0 or 1.
SEQ ID NO: 71-TNFR-2 ECD-IL10-scFv1 hole
LPAQVAFTPY APEPGSTCRL REYYDQTAQM CCSKCSPGQH AKVECTKTSD TVCDSCEDST
YTQLWNWVPE CLSCGSRCSS DQVETQACTR EQNRICTCRP GWYCALSKQE GCRLCAPLRK
CRPGFGVARP GTETSDVVCK PCAPGTESNT TSSTDICRPH QICNVVAIPG NASMDAVCTS
TSPTRSMAPG AVHLPQPVST RSQHTQPTPE PSTAPSTSEL LPMGPSPPAE GSTGDQEPKS
SAESKYGPPC PPCPAPEFLG GPSVELFPPK PKDTLYITRE PEVTCVVVDV SQEDPEVQEN
WYVDGVEVHN AKTKPREEQF NSTYRVVSVL TVLHQDWING KEYKCKVSNK GLPSSIEKTI
SKAKGQPREP QVCTLPPSQE EMTKNQVSLS CAVKGFYPSD IAVEWESNGQ PENNYKTTPP
VLDSDGSFFL VSRLTVDKSR WQEGNVESCS VMHEALHNHY TQKSLSLSLG A(GGGGSAA)ₙ₁
DIQMTQSPSS LSASVGDRVT ITCKTSQNIF ENLAWYQQKP GKAPKLLIYN ASPLQAGVPS
RFSGSGSGTD FTLTISSLQP EDFATYYCHQ YYSGYTFGPG TKLELKGGGG SGGGGSGGGG
SQVQLVESGG GVVQPGRSLR LSCAASGFTF SDYHMAWVRQ APGKGLEWVA SITLDATYTY
YRDSVRGRFT ISRDNSKNTL YLQMNSLRAE DTAVYYCARH RGESVWLDYW GQGTLVTVSS
[(GGGGSAA)ₙ₂(GPLGVR)ₙ₃(GGGGS)ₙ₄DIQ MTQSPSSLSA SVGDRVTITC KTSQNIFENL
AWYQQKPGKA PKLLIYNASP LQAGVPSRES GSGSGTDFTL TISSLQPEDF ATYYCHQYYS
GYTFGPGTKL ELKGGGGSGG GGSGGGGSQV QLVESGGGVV QPGRSLRLSC AASGFTESDY
HMAWVRQAPG KGLEWVASIT LDATYTYYRD SVRGRFTISR DNSKNTLYLQ MNSLRAEDTA
VYYCARHRGF SVWLDYWGQG TLVTVSS]ₙ₅; wherein n1 = 1, 2, 3, or 4;
n2 = 1, 2, 3, 4, or 5; n3 = 0 or 1; n4 = 0, 1, or 2; and n5 = 0,
or 1.
SEQ ID NO: 72-TNER-2 ECD-IL10-scFv2 hole
LPAQVAFTPY APEPGSTCRL REYYDQTAQM CCSKCSPGQH AKVFCTKTSD TVCDSCEDST
YTQLWNWVPE CLSCGSRCSS DQVETQACTR EQNRICTCRP GWYCALSKQE GCRLCAPLRK
CRPGFGVARP GTETSDVVCK PCAPGTESNT TSSTDICRPH QICNVVAIPG NASMDAVCTS
TSPTRSMAPG AVHLPQPVST RSQHTQPTPE PSTAPSTSEL LPMGPSPPAE GSTGDQEPKS
SAESKYGPPC PPCPAPEFLG GPSVELFPPK PKDTLYITRE PEVTCVVVDV SQEDPEVQEN
WYVDGVEVHN AKTKPREEQF NSTYRVVSVL TVLHQDWING KEYKCKVSNK GLPSSIEKTI
SKAKGQPREP QVCTLPPSQE EMTKNQVSLS CAVKGFYPSD IAVEWESNGQ PENNYKTTPP
VLDSDGSFFL VSRLTVDKSR WQEGNVESCS VMHEALHNHY TQKSLSLSLG
A(GGGGSAA)ₙ₁QVQLVESGGG VVQPGRSLRL SCAASGFTES DYHMAWVRQA PGKGLEWVAS
ITLDATYTYY RDSVRGRFTI SRDNSKNTLY LQMNSLRAED TAVYYCARHR GESVWLDYWG
QGTLVTVSSG GGGSGGGGSG GGGSDIQMTQ SPSSLSASVG DRVTITCKTS QNIFENLAWY
QQKPGKAPKL LIYNASPLQA GVPSRESGSG SGTDETLTIS SLQPEDFATY YCHQYYSGYT
FGPGTKLELK[(GGGGSAA)ₙ₂(PYAYWMR)ₙ₃(GGGGS)ₙ₄QVQ LVESGGGVVQ PGRSLRLSCA
ASGFTFSDYH MAWVRQAPGK GLEWVASITL DATYTYYRDS VRGRFTISRD NSKNTLYLQM
NSLRAEDTAV YYCARHRGES VWLDYWGQGT LVTVSSGGGG SGGGGSGGGG SDIQMTQSPS
SLSASVGDRV TITCKTSQNI FENLAWYQQK PGKAPKLLIY NASPLQAGVP SRESGSGSGT
DETLTISSLQ PEDFATYYCH QYYSGYTFGP GTKLELK]ₙ₅; wherein n1 = 1, 2, 3,
or 4; n2 = 1, 2, 3, 4, or 5; n3 = 0 or 1; n4 = 0, 1, or 2; and
n5 = 0, or 1.
SEQ ID NO: 73-TNFR-2 ECD-IL10-homodimer 1
LPAQVAFTPY APEPGSTCRL REYYDQTAQM CCSKCSPGQH AKVECTKTSD TVCDSCEDST
YTQLWNWVPE CLSCGSRCSS DQVETQACTR EQNRICTCRP GWYCALSKQE GCRLCAPLRK
CRPGFGVARP GTETSDVVCK PCAPGTESNT TSSTDICRPH QICNVVAIPG NASMDAVCTS
TSPTRSMAPG AVHLPQPVST RSQHTQPTPE PSTAPSTSFL LPMGPSPPAE GSTGDQEPKS
SAESKYGPPC PPCPAPEFLG GPSVELFPPK PKDTLMISRT PEVTCVVVDV SQEDPEVQEN
WYVDGVEVHN AKTKPREEQF NSTYRVVSVL TVLHQDWLNG KEYKCKVSNK GLPSSIEKTI
SKAKGQPREP QVYTLPPSQE EMTKNQVSLT CLVKGFYPSD IAVEWESNGQ PENNYKTTPP
VLDSDGSFFL YSRLTVDKSR WQEGNVESCS VMHEALHNHY TQKSLSLSLG
A(GGGGSAA)ₙ₁SPGQGTQSEN SCTHEPGNLP NMLRDLRDAF SRVKTFFQMK DQLDNLLLKE
SLLEDEKGYL GCQALSEMIQ FYLEEVMPQA ENQDPDIKAH VNSLGENLKT LRLRLRRCHR
SLPCENKSKA VEQVKNAFNK LQEKGIYKAM SEFDIFINYI EAYMTMKIRN
(GGGGSAA)ₙ₂(PYAYWMR)ₙ₃(GGGGS)ₙ₄DIQ MTQSPSSLSA SVGDRVTITC KTSQNIFENL
AWYQQKPGKA PKLLIYNASP LQAGVPSRES GSGSGTDETL TISSLQPEDF ATYYCHQYYS
GYTFGPGTKL ELKGGGGSGG GGSGGGGSQV QLVESGGGVV QPGRSLRLSC AASGFTFSDY
HMAWVRQAPG KGLEWVASIT LDATYTYYRD SVRGRETISR DNSKNTLYLQ MNSLRAEDTA
VYYCARHRGF SVWLDYWGQG TLVTVSS; wherein n1 = 0, 1, 2, 3, 4, or 5;
n2 = 0, 1, 2, 3, 4, or 5; n3 = 0, 1, 2, 3, 4, or 5; and n4 = 0, 1,
2, 3, 4, or 5.
SEQ ID NO: 74-TNER-2 ECD-IL10
LPAQVAFTPY APEPGSTCRL REYYDQTAQM CCSKCSPGQH AKVECTKTSD TVCDSCEDST
YTQLWNWVPE CLSCGSRCSS DQVETQACTR EQNRICTCRP GWYCALSKQE GCRLCAPLRK
CRPGFGVARP GTETSDVVCK PCAPGTESNT TSSTDICRPH QICNVVAIPG NASMDAVCTS
TSPTRSMAPG AVHLPQPVST RSQHTQPTPE PSTAPSTSFL LPMGPSPPAE GSTGDQEPKS
SAESKYGPPC PPCPAPEFLG GPSVELFPPK PKDTLMISRT PEVTCVVVDV SQEDPEVQEN
WYVDGVEVHN AKTKPREEQF NSTYRVVSVL TVLHQDWING KEYKCKVSNK GLPSSIEKTI
SKAKGQPREP QVYTLPPSQE EMTKNQVSLT CLVKGFYPSD IAVEWESNGQ PENNYKTTPP
VLDSDGSFFL YSRLTVDKSR WQEGNVESCS VMHEALHNHY TQKSLSLSLG
A(GGGGSAA)ₙ₁SPGQGTQSEN SCTHEPGNLP NMLRDLRDAF SRVKTFFQMK DQLDNLLLKE
SLLEDFKGYL GCQALSEMIQ FYLEEVMPQA ENQDPDIKAH VNSLGENLKT LRLRLRRCHR
SLPCENKSKA VEQVKNAENK LQEKGIYKAM SEFDIFINYI EAYMTMKIRN
(GGGGSAA)ₙ₂(PYAYWMR)ₙ₃(GGGGS)ₙ₄QVQ LVESGGGVVQ PGRSLRLSCA ASGFTESDYH
MAWVRQAPGK GLEWVASITL DATYTYYRDS VRGRFTISRD NSKNTLYLQM NSLRAEDTAV
YYCARHRGES VWLDYWGQGT LVTVSSGGGG SGGGGSGGGG SDIQMTQSPS SLSASVGDRV
TITCKTSQNI FENLAWYQQK PGKAPKLLIY NASPLQAGVP SRESGSGSGT DETLTISSLQ
PEDFATYYCH QYYSGYTFGP GTKLELK; wherein n1 = 0, 1, 2, 3, 4, or 5;
n2 = 1, 2, 3, 4, or 5; n3 = 0, 1, 2, 3, 4, or 5; and n4 = 0, 1, 2,
3, 4, or 5.
SEQ ID NO: 75-cleavable peptide linker
GGSLSGRSDN HGS
SEQ ID NO: 76-cleavable linker, MMP3
PYAYWMR
SEQ ID NO: 77-83-MMP-2/MMP-9 cleavable peptide linkers
GPLGVR (SEQ ID NO: 77)
PLGMWSR (SEQ ID NO: 78)
PLGLWAR (SEQ ID NO: 79)
PQGIAGQR (SEQ ID NO: 80)
PLGLAG (SEQ ID NO: 81)
LALGPR (SEQ ID NO: 82)
GGPLGMLSQS (SEQ ID NO: 83)
SEQ ID NO: 84-92-urokinase plasminogen activator (uPA) cleavable
peptide linkers
GGGGRRGGS (SEQ ID NO: 84)
TGRGPSWV (SEQ ID NO: 85)
SARGPSRW (SEQ ID NO: 86)
TARGPSFK (SEQ ID NO: 87)
TARGPSW (SEQ ID NO: 88)
GGWHTGRN (SEQ ID NO: 89)
HTGRSGAL (SEQ ID NO: 90)
PLTGRSGG (SEQ ID NO: 91)
LTGRSGA (SEQ ID NO: 92)
SEQ ID NO: 93-matriptase cleavable peptide linker
RQARVVNG (SEQ ID NO: 93)
SEQ ID NO: 94-matriptase-MMP2/9 dual cleavable peptide linker
VHMPLGFLGP RQARVVNA (SEQ ID NO: 94).
SEQ ID NO: 95
QEPKSS
SEQ ID NO: 96
(GGGGS)ₙ, wherein n = 1, 2, or 3
SEQ ID NO: 97-non-cleavable peptide linker
GSAGSAAGSG EF
SEQ ID NO: 98-non-cleavable peptide linker
(GGGGS) n1GS AGSAAGSGEF (GGGGS) 2, wherein n1 = 1, 2, or 3, and
n2 = 1, 2, or 3.
SEQ ID NO: 99-non-cleavable peptide linker
(GGGGS)ₙ₁AA (GGGGS)ₙ₂; wherein n1 = 2 or 3, and n2 = 2 or 3.
SEQ ID NO: 100-dupilumab Heavy chain-IL10-homodimer 1
EVQLVESGGG LEQPGGSLRL SCAGSGFTER DYAMTWVRQA PGKGLEWVSS ISGSGGNTYY
ADSVKGRFTI SRDNSKNTLY LQMNSLRAED TAVYYCAKDR LSITIRPRYY GLDVWGQGTT
VTVSSASTKG PSVEPLAPCS RSTSESTAAL GCLVKDYFPE PVTVSWNSGA LTSGVHTEPA
VLQSSGLYSL SSVVTVPSSS LGTKTYTCNV DHKPSNTKVD KRVESKYGPP CPPCPAPEFL
GGPSVFLFPP KPKDTLMISR TPEVTCVVVD VSQEDPEVQF NWYVDGVEVH NAKTKPREEQ
FNSTYRVVSV LTVLHQDWLN GKEYKCKVSN KGLPSSIEKT ISKAKGQPRE PQVYTLPPSQ
EEMTKNQVSL TCLVKGFYPS DIAVEWESNG QPENNYKTTP PVLDSDGSFF LYSRLTVDKS
RWQEGNVFSC SVMHEALHNH YTQKSLSLSL GAPYAYWMRS PGQGTQSENS CTHFPGNLPN
MLRDLRDAFS RVKTFFQMKD QLDNLLLKES LLEDFKGYLG CQALSEMIQF YLEEVMPQAE
NQDPDIKAHV NSLGENLKTL RLRLRRCHRS LPCENKSKAV EQVKNAFNKL QEKGIYKAMS
EFDIFINYIE AYMTMKIRN
SEQ ID NO: 101-abatacept amino acid sequence (signal peptide is
underlined)
MGVLLTQRTL LSLVLALLFP SMASMAMHVA QPAVVLASSR GIASFVCEYA SPGKATEVRV
TVLRQADSQV TEVCAATYMM GNELTELDDS ICTGTSSGNQ VNLTIQGLRA MDTGLYICKV
ELMYPPPYYL GIGNGTQIYV IDPEPCPDSD QEPKSSDKTH TSPPSPAPEL LGGSSVFLFP
PKPKDTLMIS RTPEVTCVVV DVSHEDPEVK FNWYVDGVEV HNAKTKPREE QYNSTYRVVS
VLTVLHQDWL NGKEYKCKVS NKALPAPIEK TISKAKGQPR EPQVYTIPPS RDELTKNQVS
LTCLVKGFYP SDIAVEWESN GQPENNYKTT PPVLDSDGSF FLYSKLTVDK SRWQQGNVES
CSVMHEALHN HYTQKSLSLS PGK**
SEQ ID NO: 102-abatacept-IL-10v (signal peptide is underlined)
MGVLLTQRTL LSLVLALLFP SMASMAMHVA QPAVVLASSR GIASFVCEYA SPGKATEVRV
TVLRQADSQV TEVCAATYMM GNELTELDDS ICTGTSSGNQ VNLTIQGLRA MDTGLYICKV
ELMYPPPYYL GIGNGTQIYV IDPEPCPDSD QEPKSSDKTH TSPPSPAPEL LGGSSVELFP
PKPKDTLMIS RTPEVTCVVV DVSHEDPEVK FNWYVDGVEV HNAKTKPREE QYNSTYRVVS
VLTVLHQDWL NGKEYKCKVS NKALPAPIEK TISKAKGQPR EPQVYTLPPS RDELTKNQVS
LTCLVKGFYP SDIAVEWESN GQPENNYKTT PPVLDSDGSF FLYSKLTVDK SRWQQGNVES
CSVMHEALHN HYTQKSLSLS PGAGGGGSGG GGSGGGGSSP GQGTQSENSC THFPGNLPNM
LRDLRDAFSR VKTFFQMKDQ LDNLLLKESL LEDEKGYLGC QALSEMIQFY LEEVMPQAEN
QDPDIKAHVN SLGENLKTLR LRLRRCHRSL PCENKSKAVE QVKNAFNKLQ EKGIYKAMSE
FDIFINYIEA YMTMKIRN**
SEQ ID NO: 103-abatacept-IL-10v-scFv1 (signal peptide is underlined)
MGVLLTQRTL LSLVLALLFP SMASMAMHVA QPAVVLASSR GIASFVCEYA SPGKATEVRV
TVLRQADSQV TEVCAATYMM GNELTELDDS ICTGTSSGNQ VNLTIQGLRA MDTGLYICKV
ELMYPPPYYL GIGNGTQIYV IDPEPCPDSD QEPKSSDKTH TSPPSPAPEL LGGSSVELFP
PKPKDTLMIS RTPEVTCVVV DVSHEDPEVK FNWYVDGVEV HNAKTKPREE QYNSTYRVVS
VLTVLHQDWL NGKEYKCKVS NKALPAPIEK TISKAKGQPR EPQVYTLPPS RDELTKNQVS
LTCLVKGFYP SDIAVEWESN GQPENNYKTT PPVLDSDGSF FLYSKLTVDK SRWQQGNVES
CSVMHEALHN HYTQKSLSLS PGAGGGGSGG GGSGGGGSSP GQGTQSENSC THEPGNLPNM
LRDLRDAFSR VKTFFQMKDQ LDNLLLKESL LEDFKGYLGC QALSEMIQFY LEEVMPQAEN
QDPDIKAHVN SLGENLKTLR LRLRRCHRSL PCENKSKAVE QVKNAFNKLQ EKGIYKAMSE
FDIFINYIEA YMTMKIRNGG GGSGGGGSAA GGGGGSGGGG SDIQMTQSPS SLSASVGDRV
TITCKTSQNI FENLAWYQQK PGKAPKLLIY NASPLQAGVP SRESGSGSGT DETLTISSLQ
PEDFATYYCH QYYSGYTFGP GTKLELKGGG GSGGGGSGGG GSQVQLVESG GGVVQPGRSL
RLSCAASGFT FSDYHMAWVR QAPGKGLEWV ASITLDATYT YYRDSVRGRF TISRDNSKNT
LYLQMNSLRA EDTAVYYCAR HRGFSVWLDY WGQGTLVTVS S**
SEQ ID NO: 104-abatacept-IL-10v -scFv2 (signal peptide is underlined)
MGVLLTQRTL LSLVLALLFP SMASMAMHVA QPAVVLASSR GIASFVCEYA SPGKATEVRV
TVLRQADSQV TEVCAATYMM GNELTELDDS ICTGTSSGNQ VNLTIQGLRA MDTGLYICKV
ELMYPPPYYL GIGNGTQIYV IDPEPCPDSD QEPKSSDKTH TSPPSPAPEL LGGSSVELEP
PKPKDTLMIS RTPEVTCVVV DVSHEDPEVK FNWYVDGVEV HNAKTKPREE QYNSTYRVVS
VLTVLHQDWL NGKEYKCKVS NKALPAPIEK TISKAKGQPR EPQVYTLPPS RDELTKNQVS
LTCLVKGFYP SDIAVEWESN GQPENNYKTT PPVLDSDGSF FLYSKLTVDK SRWQQGNVES
CSVMHEALHN HYTQKSLSLS PGAGGGGSGG GGSGGGGSSP GQGTQSENSC THFPGNLPNM
LRDLRDAFSR VKTFFQMKDQ LDNLLLKESL LEDEKGYLGC QALSEMIQFY LEEVMPQAEN
QDPDIKAHVN SLGENLKTLR LRLRRCHRSL PCENKSKAVE QVKNAFNKLQ EKGIYKAMSE
FDIFINYIEA YMTMKIRNGG GGSGGGGSAA GGGGGSGGGG SQVQLVESGG GVVQPGRSLR
LSCAASGFTF SDYHMAWVRQ APGKGLEWVA SITLDATYTY YRDSVRGRET ISRDNSKNTL
YLQMNSLRAE DTAVYYCARH RGESVWLDYW GQGTLVTVSS GGGGSGGGGS GGGGSDIQMT
QSPSSLSASV GDRVTITCKT SQNIFENLAW YQQKPGKAPK LLIYNASPLQ AGVPSRESGS
GSGTDFTLTI SSLQPEDFAT YYCHQYYSGY TFGPGTKLEL K**
SEQ ID NO: 105-abatacept-scFv1-IL10v (signal peptide is underlined)
MGVLLTQRTL LSLVLALLFP SMASMAMHVA QPAVVLASSR GIASFVCEYA SPGKATEVRV
TVLRQADSQV TEVCAATYMM GNELTELDDS ICTGTSSGNQ VNLTIQGLRA MDTGLYICKV
ELMYPPPYYL GIGNGTQIYV IDPEPCPDSD QEPKSSDKTH TSPPSPAPEL LGGSSVELFP
PKPKDTLMIS RTPEVTCVVV DVSHEDPEVK FNWYVDGVEV HNAKTKPREE QYNSTYRVVS
VLTVLHQDWL NGKEYKCKVS NKALPAPIEK TISKAKGQPR EPQVYTLPPS RDELTKNQVS
LTCLVKGFYP SDIAVEWESN GQPENNYKTT PPVLDSDGSF FLYSKLTVDK SRWQQGNVES
CSVMHEALHN HYTQKSLSLS PGAGGGGSGG GGSGGGGSDI QMTQSPSSLS ASVGDRVTIT
CKTSQNIFEN LAWYQQKPGK APKLLIYNAS PLQAGVPSRF SGSGSGTDET LTISSLQPED
FATYYCHQYY SGYTFGPGTK LELKGGGGSG GGGSGGGGSQ VQLVESGGGV VQPGRSLRLS
CAASGFTESD YHMAWVRQAP GKGLEWVASI TLDATYTYYR DSVRGRETIS RDNSKNTLYL
QMNSLRAEDT AVYYCARHRG FSVWLDYWGQ GTLVTVSSGG GGSGGGGSAA GGGGGSGGGG
SSPGQGTQSE NSCTHEPGNL PNMLRDLRDA FSRVKTFFQM KDQLDNLLLK ESLLEDEKGY
LGCQALSEMI QFYLEEVMPQ AENQDPDIKA HVNSLGENLK TLRLRLRRCH RSLPCENKSK
AVEQVKNAFN KLQEKGIYKA MSEFDIFINY IEAYMTMKIR N**
SEQ ID NO: 106-abatacept-scFv2-IL-10v (signal peptide is underlined)
MGVLLTQRTL LSLVLALLFP SMASMAMHVA QPAVVLASSR GIASFVCEYA SPGKATEVRV
TVLRQADSQV TEVCAATYMM GNELTELDDS ICTGTSSGNQ VNLTIQGLRA MDTGLYICKV
ELMYPPPYYL GIGNGTQIYV IDPEPCPDSD QEPKSSDKTH TSPPSPAPEL LGGSSVELEP
PKPKDTLMIS RTPEVTCVVV DVSHEDPEVK FNWYVDGVEV HNAKTKPREE QYNSTYRVVS
VLTVLHQDWL NGKEYKCKVS NKALPAPIEK TISKAKGQPR EPQVYTLPPS RDELTKNQVS
LTCLVKGFYP SDIAVEWESN GQPENNYKTT PPVLDSDGSF FLYSKLTVDK SRWQQGNVES
CSVMHEALHN HYTQKSLSLS PGAGGGGSGG GGSGGGGSQV QLVESGGGVV QPGRSLRLSC
AASGFTFSDY HMAWVRQAPG KGLEWVASIT LDATYTYYRD SVRGRETISR DNSKNTLYLQ
MNSLRAEDTA VYYCARHRGF SVWLDYWGQG TLVTVSSGGG GSGGGGSGGG GSDIQMTQSP
SSLSASVGDR VTITCKTSQN IFENLAWYQQ KPGKAPKLLI YNASPLQAGV PSRESGSGSG
TDFTLTISSL QPEDFATYYC HQYYSGYTFG PGTKLELKGG GGSGGGGSAA GGGGGSGGGG
SSPGQGTQSE NSCTHEPGNL PNMLRDLRDA FSRVKTFFQM KDQLDNLLLK ESLLEDEKGY
LGCQALSEMI QFYLEEVMPQ AENQDPDIKA HVNSLGENLK TLRLRLRRCH RSLPCENKSK
AVEQVKNAFN KLQEKGIYKA MSEFDIFINY IEAYMTMKIR N**
SEQ ID NO: 107-abatacept-IL-10v-RF (signal peptide is underlined)
MGVLLTQRTL LSLVLALLFP SMASMAMHVA QPAVVLASSR GIASFVCEYA SPGKATEVRV
TVLRQADSQV TEVCAATYMM GNELTELDDS ICTGTSSGNQ VNLTIQGLRA MDTGLYICKV
ELMYPPPYYL GIGNGTQIYV IDPEPCPDSD QEPKSSDKTH TSPPSPAPEL LGGSSVELFP
PKPKDTLMIS RTPEVTCVVV DVSHEDPEVK FNWYVDGVEV HNAKTKPREE QYNSTYRVVS
VLTVLHQDWL NGKEYKCKVS NKALPAPIEK TISKAKGQPR EPQVYTLPPS RDELTKNQVS
LTCLVKGFYP SDIAVEWESN GQPENNYKTT PPVLDSDGSF FLYSKLTVDK SRWQQGNVES
CSVMHEALHN RFTQKSLSLS PGAGGGGSGG GGSGGGGSSP GQGTQSENSC THEPGNLPNM
LRDLRDAFSR VKTFFQMKDQ LDNLLLKESL LEDFKGYLGC QALSEMIQFY LEEVMPQAEN
QDPDIKAHVN SLGENLKTLR LRLRRCHRSL PCENKSKAVE QVKNAFNKLQ EKGIYKAMSE
EDIFINYIEA YMTMKIRN**
SEQ ID NO: 108-light chain of antagonist antibody against trem-1
(signal peptide is underlined)
MDMRVPAQLL GLLLLWLPGA KCDIVLTQSP DSLAVSLGER ATINCRASES VDTEDYSELH
WYQQKPGQPP KLLIYRASNL ESGVPDRFSG SGSGTDETLT ISSLQAEDVA VYYCQQSNED
PYTFGQGTKL EIKRTVAAPS VFIFPPSDEQ LKSGTASVVC LLNNFYPREA KVQWKVDNAL
QSGNSQESVT EQDSKDSTYS LSSTLTLSKA DYEKHKVYAC EVTHQGLSSP VTKSENRGEC**
SEQ ID NO: 109-heavy chain of antagonist antibody against trem-1
(signal peptide is underlined)
MGVKVLFALI CIAVAEAEVQ LVESGGGLVQ PGGSLKLSCA ASGFTESTYA MHWVRQASGK
GLEWVGRIRT KSSNYATYYA ASVKGRFTIS RDDSKNTAYL QMNSLKTEDT AVYYCTRDMG
IRRQFAYWGQ GTLVTVSSAS TKGPSVEPLA PCSRSTSEST AALGCLVKDY FPEPVTVSWN
SGALTSGVHT FPAVLQSSGL YSLSSVVTVP SSSLGTKTYT CNVDHKPSNT KVDKRVESKY
GPPCPPCPAP EFLGGPSVEL FPPKPKDTLM ISRTPEVTCV VVDVSQEDPE VQFNWYVDGV
EVHNAKTKPR EEQFNSTYRV VSVLTVLHQD WINGKEYKCK VSNKGLPSSI EKTISKAKGQ
PREPQVYTLP PSQEEMTKNQ VSLTCLVKGF YPSDIAVEWE SNGQPENNYK TTPPVLDSDG
SFFLYSRLTV DKSRWQEGNV FSCSVMHEAL HNHYTQKSLS LSLGK**
SEQ ID NO: 110-trem-1-IL-10v F111S-scFv1-cleavable (signal peptide
is underlined)
MGVKVLFALI CIAVAEAEVQ LVESGGGLVQ PGGSLKLSCA ASGFTFSTYA MHWVRQASGK
GLEWVGRIRT KSSNYATYYA ASVKGRFTIS RDDSKNTAYL QMNSLKTEDT AVYYCTRDMG
IRRQFAYWGQ GTLVTVSSAS TKGPSVEPLA PCSRSTSEST AALGCLVKDY FPEPVTVSWN
SGALTSGVHT FPAVLQSSGL YSLSSVVTVP SSSLGTKTYT CNVDHKPSNT KVDKRVESKY
GPPCPPCPAP EFLGGPSVEL FPPKPKDTLM ISRTPEVTCV VVDVSQEDPE VQFNWYVDGV
EVHNAKTKPR EEQFNSTYRV VSVLTVLHQD WLNGKEYKCK VSNKGLPSSI EKTISKAKGQ
PREPQVYTLP PSQEEMTKNQ VSLTCLVKGF YPSDIAVEWE SNGQPENNYK TTPPVLDSDG
SFFLYSRLTV DKSRWQEGNV FSCSVMHEAL HNHYTQKSLS LSLGAGGGGS GGGGSGGGGS
SPGQGTQSEN SCTHEPGNLP NMLRDLRDAF SRVKTFFQMK DQLDNLLLKE SLLEDEKGYL
GCQALSEMIQ FYLEEVMPQA ENQDPDIKAH VNSLGENLKT LRLRLRRCHR SLPCENKSKA
VEQVKNAFNK LQEKGIYKAM SEFDIFINYI EAYMTMKIRN GGGGSGGGGS GPLGVRGGGG
SGGGGSDIQM TQSPSSLSAS VGDRVTITCK TSQNIFENLA WYQQKPGKAP KLLIYNASPL
QAGVPSRFSG SGSGTDFTLT ISSLQPEDFA TYYCHQYYSG YTFGPGTKLE LKGGGGSGGG
GSGGGGSQVQ LVESGGGVVQ PGRSLRLSCA ASGFTFSDYH MAWVRQAPGK GLEWVASITL
DATYTYYRDS VRGRFTISRD NSKNTLYLQM NSLRAEDTAV YYCARHRGES VWLDYWGQGT
LVTVSS**
SEQ ID NO: 111-trem-1-scFv1-IL10v (signal peptide is underlined)
MGVKVLFALI CIAVAEAEVQ LVESGGGLVQ PGGSLKLSCA ASGFTESTYA MHWVRQASGK
GLEWVGRIRT KSSNYATYYA ASVKGRFTIS RDDSKNTAYL QMNSLKTEDT AVYYCTRDMG
IRRQFAYWGQ GTLVTVSSAS TKGPSVEPLA PCSRSTSEST AALGCLVKDY FPEPVTVSWN
SGALTSGVHT FPAVLQSSGL YSLSSVVTVP SSSLGTKTYT CNVDHKPSNT KVDKRVESKY
GPPCPPCPAP EFLGGPSVEL FPPKPKDTLM ISRTPEVTCV VVDVSQEDPE VQFNWYVDGV
EVHNAKTKPR EEQFNSTYRV VSVLTVLHQD WLNGKEYKCK VSNKGLPSSI EKTISKAKGQ
PREPQVYTLP PSQEEMTKNQ VSLTCLVKGF YPSDIAVEWE SNGQPENNYK TTPPVLDSDG
SFFLYSRLTV DKSRWQEGNV FSCSVMHEAL HNHYTQKSLS LSLGAGGGGS GGGGSGGGGS
DIQMTQSPSS LSASVGDRVT ITCKTSQNIF ENLAWYQQKP GKAPKLLIYN ASPLQAGVPS
RFSGSGSGTD FTLTISSLQP EDFATYYCHQ YYSGYTFGPG TKLELKGGGG SGGGGSGGGG
SQVQLVESGG GVVQPGRSLR LSCAASGFTF SDYHMAWVRQ APGKGLEWVA SITLDATYTY
YRDSVRGRET ISRDNSKNTL YLQMNSLRAE DTAVYYCARH RGESVWLDYW GQGTLVTVSS
GGGGSGGGGS AAGGGGGSGG GGSSPGQGTQ SENSCTHEPG NLPNMLRDLR DAFSRVKTFF
QMKDQLDNLL LKESLLEDFK GYLGCQALSE MIQFYLEEVM PQAENQDPDI KAHVNSLGEN
LKTLRLRLRR CHRSLPCENK SKAVEQVKNA ENKLQEKGIY KAMSEFDIFI NYIEAYMTMK
IRN**
SEQ ID NO: 112-trem-1-IL-10v-RF (signal peptide is underlined)
MGVKVLFALI CIAVAEAEVQ LVESGGGLVQ PGGSLKLSCA ASGFTESTYA MHWVRQASGK
GLEWVGRIRT KSSNYATYYA ASVKGRFTIS RDDSKNTAYL QMNSLKTEDT AVYYCTRDMG
IRRQFAYWGQ GTLVTVSSAS TKGPSVEPLA PCSRSTSEST AALGCLVKDY FPEPVTVSWN
SGALTSGVHT FPAVLQSSGL YSLSSVVTVP SSSLGTKTYT CNVDHKPSNT KVDKRVESKY
GPPCPPCPAP EFLGGPSVEL FPPKPKDTLM ISRTPEVTCV VVDVSQEDPE VQFNWYVDGV
EVHNAKTKPR EEQENSTYRV VSVLTVLHQD WINGKEYKCK VSNKGLPSSI EKTISKAKGQ
PREPQVYTLP PSQEEMTKNQ VSLTCLVKGF YPSDIAVEWE SNGQPENNYK TTPPVLDSDG
SFFLYSRLTV DKSRWQEGNV FSCSVMHEAL HNRFTQKSLS LSLGAGGGGS GGGGSGGGGS
SPGQGTQSEN SCTHEPGNLP NMLRDLRDAF SRVKTFFQMK DQLDNLLLKE SLLEDFKGYL
GCQALSEMIQ FYLEEVMPQA ENQDPDIKAH VNSLGENLKT LRLRLRRCHR SLPCENKSKA
VEQVKNAFNK LQEKGIYKAM SEFDIFINYI EAYMTMKIRN**
SEQ ID NO: 113 trem-1-IL-10v F111S-scFv1-MMP3 cleavable, IGG1-
LALA_G237A-YTE (signal peptide is underlined)
MGVKVLFALI CIAVAEAEVQ LVESGGGLVQ PGGSLKLSCA ASGFTESTYA MHWVRQASGK
GLEWVGRIRT KSSNYATYYA ASVKGRFTIS RDDSKNTAYL QMNSLKTEDT AVYYCTRDMG
IRRQFAYWGQ GTLVTVSSAS TKGPSVEPLA PCSRSTSEST AALACLVKDY FPEPVTVSWN
SGALTSGVHT FPAVLQSSGL YSLSSVVTVP SSSLGTKTYT CNVDHKPSNT KVDKRVESKY
GPPCPPCPAP EFLGGPSVEL FPPKPKDTLM ISRTPEVTCV VVDVSQEDPE VQFNWYVDGV
EVHNAKTKPR EEQFNSTYRV VSVLTVLHQD WLNGKEYKCK VSNKGLPSSI EKTISKAKGQ
PREPQVYTLP PSQEEMTKNQ VSLTCLVKGF YPSDIAVEWE SNGQPENNYK TTPPVLDSDG
SFFLYSRLTV DKSRWQEGNV FSCSVMHEAL HNHYTQKSLS LSLGAGGGGS GGGGSGGGGS
SPGQGTQSEN SCTHFPGNLP NMLRDLRDAF SRVKTFFQMK DQLDNLLLKE SLLEDEKGYL
GCQALSEMIQ FYLEEVMPQA ENQDPDIKAH VNSLGENLKT LRLRLRRCHR SLPCENKSKA
VEQVKNAFNK LQEKGIYKAM SEFDIFINYI EAYMTMKIRN GGGGSGGGGS PYAYWMRGGG
GSGGGGSDIQ MTQSPSSLSA SVGDRVTITC KTSQNIFENL AWYQQKPGKA PKLLIYNASP
LQAGVPSRES GSGSGTDETL TISSLQPEDF ATYYCHQYYS GYTFGPGTKL ELKGGGGSGG
GGSGGGGSQV QLVESGGGVV QPGRSLRLSC AASGFTESDY HMAWVRQAPG KGLEWVASIT
LDATYTYYRD SVRGRFTISR DNSKNTLYLQ MNSLRAEDTA VYYCARHRGF SVWLDYWGQG
TLVTVSS**
SEQ ID NO: 114-IGG1-LALA_G237A-YTE, MAdCAM-IL-10v-scFv2-cleavable
MMP3 (signal peptide is underlined)
MGVKVLFALI CIAVAEAQVQ LVQSGAEVKK PGASVKVSCK ASGYTFTSYG INWVRQAPGQ
GLEWMGWISV YSGNTNYAQK VQGRVTMTAD TSTSTAYMDL RSLRSDDTAV YYCAREGSSS
SGDYYYGMDV WGQGTTVTVS SASTKGPSVF PLAPSSKSTS GGTAALACLV KDYFPEPVTV
SWNSGALTSG VHTFPAVLQS SGLYSLSSVV TVPSSSLGTQ TYICNVNHKP SNTKVDKKAE
PKSCDKTHTC PPCPAPEAAG GPSVELFPPK PKDTLYITRE PEVTCVVVDV SHEDPEVKEN
WYVDGVEVHN AKTKPREEQY NSTYRVVSVL TVLHQDWLNG KEYKCKVSNK ALPAPIEKTI
SKAKGQPREP QVYTLPPSRE EMTKNQVSLT CLVKGFYPSD IAVEWESNGQ PENNYKTTPP
VLDSDGSFFL YSKLTVDKSR WQQGNVESCS VMHEALHNHY TQKSLSLSPG GGGSGGGGSG
GGGSSPGQGT QSENSCTHEP GNLPNMLRDL RDAFSRVKTF FQMKDQLDNL LLKESLLEDE
KGYLGCQALS EMIQFYLEEV MPQAENQDPD IKAHVNSLGE NLKTLRLRLR RCHRSLPCEN
KSKAVEQVKN AFNKLQEKGI YKAMSEFDIF INYIEAYMTM KIRNAGGGGS GGGGSPYAYW
MRGGGGSGGG GSQVQLVESG GGVVQPGRSL RLSCAASGFT FSDYHMAWVR QAPGKGLEWV
ASITLDATYT YYRDSVRGRF TISRDNSKNT LYLQMNSLRA EDTAVYYCAR HRGESVWLDY
WGQGTLVTVS SGGGGSGGGG SGGGGSDIQM TQSPSSLSAS VGDRVTITCK TSQNIFENLA
WYQQKPGKAP KLLIYNASPL QAGVPSRESG SGSGTDETLT ISSLQPEDFA TYYCHQYYSG
YTFGPGTKLE LK
SEQ ID NO: 115-IGG1-LALA_G237A-YTE, MAdCAM-scFv2-IL-10v-cleavable
MMP3
QVQLVQSGAE VKKPGASVKV SCKASGYTFT SYGINWVRQA PGQGLEWMGW ISVYSGNTNY
AQKVQGRVTM TADTSTSTAY MDLRSLRSDD TAVYYCAREG SSSSGDYYYG MDVWGQGTTV
TVSSASTKGP SVEPLAPSSK STSGGTAALA CLVKDYFPEP VTVSWNSGAL TSGVHTFPAV
LQSSGLYSLS SVVTVPSSSL GTQTYICNVN HKPSNTKVDK KAEPKSCDKT HTCPPCPAPE
AAGGPSVELF PPKPKDTLYI TREPEVTCVV VDVSHEDPEV KENWYVDGVE VHNAKTKPRE
EQYNSTYRVV SVLTVLHQDW LNGKEYKCKV SNKALPAPIE KTISKAKGQP REPQVYTLPP
SREEMTKNQV SLTCLVKGFY PSDIAVEWES NGQPENNYKT TPPVLDSDGS FFLYSKLTVD
KSRWQQGNVF SCSVMHEALH NHYTQKSLSL SPGGGGSGGG GSGGGGSQVQ LVESGGGVVQ
PGRSLRLSCA ASGFTFSDYH MAWVRQAPGK GLEWVASITL DATYTYYRDS VRGRFTISRD
NSKNTLYLQM NSLRAEDTAV YYCARHRGES VWLDYWGQGT LVTVSSGGGG SGGGGSGGGG
SDIQMTQSPS SLSASVGDRV TITCKTSQNI FENLAWYQQK PGKAPKLLIY NASPLQAGVP
SRESGSGSGT DETLTISSLQ PEDFATYYCH QYYSGYTEGP GTKLELKAGG GGSPYAYWMR
GGGGSGGSPG QGTQSENSCT HFPGNLPNML RDLRDAFSRV KTFFQMKDQL DNLLLKESLL
EDFKGYLGCQ ALSEMIQFYL EEVMPQAENQ DPDIKAHVNS LGENLKTLRL RLRRCHRSLP
CENKSKAVEQ VKNAFNKLQE KGIYKAMSEF DIFINYIEAY MTMKIRNA
SEQ ID NO: 116-MadCam Antibody Light Chain
DIVMTQTPLS LSVTPGQPAS ISCKSSQSLL HTDGTTYLYW YLQKPGQPPQ LLIYEVSNRE
SGVPDRESGS GSGTDFTLKI SRVEAEDVGI YYCMQNIQLP WTFGQGTKVE IKRTVAAPSV
FIFPPSDEQL KSGTASVVCL LNNFYPREAK VQWKVDNALQ SGNSQESVTE QDSKDSTYSL
SSTLTLSKAD YEKHKVYACE VTHQGLSSPV TKSENRGEC

1. A prodrug comprising a cytokine moiety, a masking moiety, and a carrier moiety, wherein
the masking moiety binds to the cytokine moiety and inhibits a biological activity of the cytokine moiety;
the cytokine moiety comprises an interleukin-10 (IL-10) agonist polypeptide or a transforming growth factor beta (TGF-β) agonist polypeptide and is fused to the carrier moiety or the masking moiety;
the carrier moiety binds to an antigen on the surface of an immune cell, wherein the immune cell expresses a receptor for the cytokine moiety; and
the masking moiety is fused to the cytokine moiety or to the carrier moiety, optionally through a peptide linker.

2. The prodrug of claim 1, wherein the IL-10 agonist polypeptide comprises SEQ ID NO: 1, 2, or 3, or an amino acid sequence that is at least 90% identical to SEQ ID NO: 1, 2, or 3.

3. The prodrug of claim 1, wherein the TGF-β agonist polypeptide is a TGF-β1 agonist polypeptide, a TGF-β2 agonist polypeptide, or a TGF-β3 agonist polypeptide.

4. The prodrug of claim 3, wherein the TGF-β agonist polypeptide comprises SEQ ID NO: 7, or an amino acid sequence that is at least 90% identical to SEQ ID NO: 7.

5. The prodrug of any one of claims 1-4, wherein the cytokine moiety is fused to the carrier moiety directly or via a non-cleavable or cleavable peptide linker and the masking moiety is fused to the carrier moiety directly or via a non-cleavable or cleavable peptide linker.

6. The prodrug of claim 5, wherein the prodrug further comprises a second cytokine moiety fused to the C-terminus of the cytokine moiety.

7. The prodrug of claim 5 or 6, wherein the prodrug further comprises a second masking moiety fused to the C-terminus of the masking moiety.

8. The prodrug of any one of claims 1-4, wherein two cytokine moieties are separately fused to the carrier moiety through cleavable peptide linkers, wherein said cleavable linker comprises 4, 5, 6, 7, 8, 9, or 10 amino acids, optionally comprising SEQ ID NO: 77.

9. The prodrug of any one of claims 1-4, wherein two cytokine moieties are separately fused to the carrier moiety directly or via non-cleavable peptide linkers and two masking moieties are separately fused to the two cytokine moieties directly or via non-cleavable or cleavable peptide linkers.

10. The prodrug of any one of claims 1-4, wherein two cytokine moieties are separately fused to the carrier moiety directly or via non-cleavable peptide linkers and one masking moiety is fused to one of the two cytokine moieties directly or via a non-cleavable or a cleavable peptide linker.

11. The prodrug of any one of claims 1-4, wherein two masking moieties are separately fused to the carrier moiety directly or via non-cleavable peptide linkers and two cytokine moieties are separately fused to the two masking moieties directly or via non-cleavable peptide linkers.

12. The prodrug of any one of claims 1-4, wherein the cytokine moiety is fused to the carrier moiety directly or via a non-cleavable peptide linker and the masking moiety is fused to the carrier moiety directly or via a non-cleavable peptide linker, and a second cytokine moiety is fused to the C-terminus of the masking moiety directly or via a non-cleavable peptide linker.

13. The prodrug of any one of the preceding claims, wherein the carrier moiety comprises 1) an antibody or antigen-binding fragment thereof, or 2) an antibody Fc domain and two antigen-binding moieties, which are fused directly or via a non-cleavable peptide linker to the N-terminus or the C-terminus of the Fc domain.

14. The prodrug of claim 13, wherein the antibody or antigen-binding fragment thereof binds to an antigen expressed on the surface of an immune cell.

15. The prodrug of claim 14, wherein the immune cell is selected from an NK cell, a T cell, a B cell, and a macrophage and expresses a cell surface receptor for the cytokine moiety.

16. The prodrug of any one of claims 13-15, wherein the Fc domain optionally comprises knobs-into-holes mutations.

17. The prodrug of any one of claims 13-15, wherein the Fc domain or the Fc domain of the antibody optionally comprises RF mutations, wherein the RF mutations reduce or eliminate binding of the Fc domain to a protein A affinity resin.

18. The prodrug of claim 17, wherein the RF mutation is selected from H371R/Y372F (numbering according to SEQ ID NO: 107) or H453R/Y454F (numbering according to SEQ ID NO: 112).

19. The prodrug of any one of claims 13-15, 17 and 18, wherein the carrier moiety comprises an antibody or antigen-binding fragment thereof that binds to an antigen selected from IL-1 receptor accessory protein (IL1RAP), IL-1 receptor (IL-1RI), a human IL-3 receptor, IL-4 receptor α chain (IL-4Rα), IL-5 receptor α chain (IL-5Rα), IL-6 receptor α chain (IL-6Rα), a human IL-9 receptor, a human IL-13 receptor, a human IL-17 receptor, a human IL-23 receptor, a human IL-31 receptor, a human IL-33 receptor, a receptor for thymic stromal lymphopoietin (TSLP), CD20, CD25, BCMA, CD40, CD80, CD86, Trem-1, CSF-1R, OX40, 4-1BB, TNF-alpha receptor 1 (TNFR-1), TNF-alpha receptor 1 (TNFR-2), a receptor for B lymphocyte stimulator (BLyS), mucosal addressin cell adhesion molecule 1 (MAdCAM-1), and an Interferon-alpha receptor.

20. The prodrug of any one of claims 13-15, 17 and 18, wherein the carrier moiety comprises an antibody or antigen-binding fragment that comprises the same heavy and light chain complementarity-determining regions (CDRs), the same heavy and light variable domains, or the same heavy and light chains, as an antibody selected from canakinumab, adalimumab, CDP-571, infliximab, rontalizumab, sifalimumab, olokizumab (CDP6038), elsilimomab, BMS-945429 (ALD518), sirukumab (CNTO 136), levilimab (BCD-089), siltuximab, secukinumab, ixekizumab, ustekinuma, guselkumab, and tildrakizumab.

21. The prodrug of any one of claims 13-19, wherein said carrier moiety comprises an anti-IL-4 receptor α chain (IL-4Rα) antibody or an antigen-binding fragment thereof, which comprises
light chain CDRs derived from SEQ ID NO: 11 and heavy chain CDRs derived from SEQ ID NO: 12; or
a light chain variable domain with an amino acid sequence of SEQ ID NO: 13 or at least 95% identical thereto, and a heavy chain variable domain with an amino acid sequence of SEQ ID NO: 14 or at least 95% identical thereto.

22. The prodrug of any one of claims 13-19, wherein said carrier moiety comprises an anti-IL-5 receptor α chain (IL-5Rα) antibody or an antigen-binding fragment thereof, which comprises a light chain variable domain with an amino acid sequence of SEQ ID NO: 15 or at least 95% identical thereto, and a heavy chain variable domain with an amino acid sequence of SEQ ID NO: 16 or at least 95% identical thereto.

23. The prodrug of any one of claims 13-19, wherein said carrier moiety comprises an anti-IL-6 receptor α chain (IL-6Rα) antibody or a binding fragment thereof, which comprises
a light chain variable domain with an amino acid sequence of SEQ ID NO: 17 or at least 95% identical thereto, and a heavy chain variable domain with an amino acid sequence of SEQ ID NO: 18 or at least 95% identical thereto; or
a light chain variable domain with an amino acid sequence of SEQ ID NO: 19 or at least 95% identical thereto, and a heavy chain variable domain with an amino acid sequence of SEQ ID NO: 19 or at least 95% identical thereto.

24. The prodrug of any one of claims 13-19, wherein said carrier moiety comprises an anti-Trem-1 antibody or a fragment thereof, which comprises a light chain variable domain with an amino acid sequence of SEQ ID NO: 21 or at least 95% identical thereto, and a heavy chain variable domain with an amino acid sequence of SEQ ID NO: 22 or at least 95% identical thereto.

25. The prodrug of any one of claims 13-19, wherein said carrier moiety comprises an anti-CD86 antibody or a fragment thereof, which comprises a light chain variable domain with an amino acid sequence of SEQ ID NO: 23 or at least 95% identical thereto, and a heavy chain variable domain with an amino acid sequence of SEQ ID NO: 24 or at least 95% identical thereto.

26. The prodrug of any one of claims 13-19, wherein said carrier moiety comprises an extracellular domain of CTLA-4 or a functional analog thereof, which comprises an amino acid sequence of SEQ ID NO: 25 or 61 or at least 95% identical thereto.

27. The prodrug of any one of claims 13-19, wherein said carrier moiety comprises an anti-interferon alpha receptor 1 (IFNRA-1) antibody or antigen-binding fragment thereof, which comprises a light chain variable domain with an amino acid sequence of SEQ ID NO: 52 or at least 95% identical thereto, and a heavy chain variable domain with an amino acid sequence of SEQ ID NO: 51 or at least 95% identical thereto.

28. The prodrug of any one of claims 13-19, wherein said carrier moiety comprises an anti-CD86 antibody or a fragment thereof, which comprises a light chain variable domain with an amino acid sequence of SEQ ID NO: 23 or at least 95% identical thereto, and a heavy chain variable domain with an amino acid sequence of SEQ ID NO: 24 or at least 95% identical thereto.

29. The prodrug of any one of claims 1-28, comprising a peptide linker that is cleavable by one or more proteases located at a site of inflammation or an autoimmune disease, optionally selected comprising a substrate sequence of urokinase-type plasminogen activator (uPA), matrix metallopeptidase (MT1-MMP), matrix metallopeptidase 2 (MMP2), MMP3, MMP9, matriptase, legumain, plasmin, TMPRSS-3/4, cathepsin, caspase, human neutrophil elastase, beta-secretase, or PSA, or (i) both uPA and MMP2, (ii) both uPA and MMP9, or (iii) matriptase, MMP2 and MMP9.

30. The prodrug of claim 29, wherein the cleavable peptide linker comprises an amino acid sequence selected from SEQ ID NOs: 75-95.

31. The prodrug of any one of claims 1-28, comprising a non-cleavable peptide linker comprising an amino acid sequence selected from SEQ ID NOs: 95-99.

32. The prodrug of any one of the preceding claims, wherein the masking moiety inhibits the binding of the cytokine moiety to its receptor on the surface of a cell.

33. The prodrug of any one of claims 1-32, wherein the masking moiety comprises
an extracellular domain of IL-10 receptor α chain (IL-10Rα-ECD), an analog of IL-10Rα-ECD, or an antibody against human IL-10 or a binding fragment thereof, or
SEQ ID NO: 4, 5, or 6, or an amino acid sequence that is at least 95% identical thereto.

34. The prodrug of any one of claims 1-32, wherein the masking moiety comprises an extracellular domain of TGF-β Receptor II (TGFRII-ECD), an analog of TGFRII-ECD, or an antibody against human TGF-β or a binding fragment thereof,
SEQ ID NO: 10, or an amino acid sequence that is at least 95% identical thereto, or
a scFv that binds to human TGF-β, optionally wherein the scFv comprises a VH domain with an amino acid sequence of SEQ ID NO: 9 or at least 95% identical thereto, and a VL domain with an amino acid sequence of SEQ ID NO: 8 or at least 95% identical thereto.

35. The prodrug of any one of claims 1-34, comprising two identical light chains and a first heavy chain polypeptide chain and a second heavy chain polypeptide chain, wherein
the light chain comprises SEQ ID NO: 26 or an amino acid sequence at least 95% identical thereto,
the first heavy chain polypeptide chain comprises SEQ ID NO: 27 or 28, or an amino acid sequence at least 95% identical thereto, and
the second heavy chain polypeptide chain comprises SEQ ID NO: 29 or 30, or an amino acid sequence at least 95% identical thereto.

36. The prodrug of any one of claims 1-34, comprising two identical light chains and two identical heavy chains; wherein
the light chain comprises SEQ ID NO: 26 or an amino acid sequence at least 95% identical thereto, and
the heavy chain comprises SEQ ID NO: 31, 32, 33, 34, or 100 or an amino acid sequence at least 95% identical thereto.

37. The prodrug of any one of claims 1-34, comprising two identical light chains and a first heavy chain polypeptide chain and a second heavy chain polypeptide chain; wherein
the light chain comprises SEQ ID NO: 35 or an amino acid sequence at least 95% identical thereto,
the first heavy chain polypeptide chain comprises SEQ ID NO: 36 or an amino acid sequence at least 95% identical thereto, and
the second heavy chain polypeptide chain comprises SEQ ID NO: 37 or 38, or an amino acid sequence at least 95% identical there.

38. The prodrug of any one of claims 1-34, comprising two identical light chains and two identical heavy chains, wherein
the light chain comprises SEQ ID NO: 35 or an amino acid sequence at least 95% identical thereto, and
the heavy chain comprises SEQ ID NO: 39, 40, 41 or 42, or an amino acid sequence at least 95% identical thereto.

39. The prodrug of any one of claims 1-34, comprising two identical light chains and a first heavy chain polypeptide chain and a second heavy chain polypeptide chain, wherein
the light chain comprises SEQ ID NO: 43 or an amino acid sequence at least 95% identical thereto SEQ ID NO: 43,
the first heavy chain polypeptide chain comprises SEQ ID NO: 44 or an amino acid sequence at least 95% identical thereto, and
the second heavy chain polypeptide chain comprises SEQ ID NO: 45 or 46, or an amino acid sequence at least 95% identical thereto.

40. The prodrug of any one of claims 1-34, comprising two identical light chains and two identical heavy chains, wherein
the light chain comprises SEQ ID NO: 43 or said at least 95% identical thereto, and
the heavy chain comprises SEQ ID NO: 47, 48, 49 or 50, or an amino acid sequence at least 95% identical thereto.

41. The prodrug of any one of claims 1-34, comprising two identical light chains and a first heavy chain polypeptide chain and a second heavy chain polypeptide chain,
the light chain comprises SEQ ID NO: 53 or an amino acid sequence at least 95% identical thereto,
the first heavy chain polypeptide chain comprises SEQ ID NO: 54 or an amino acid sequence at least 95% identical thereto, and
the second heavy chain polypeptide chain comprises SEQ ID NO: 55 or 56, or an amino acid sequence at least 95% identical thereto.

42. The prodrug of any one of claims 1-34, comprising two identical light chains and two identical heavy chains, wherein
the light chain comprises SEQ ID NO: 53 or an amino acid sequence at least 95% identical thereto, and
the heavy chain comprises SEQ ID NO: 57, 58, 59 or 60, or an amino acid sequence at least 95% identical thereto.

43. The prodrug of any one of claims 1-34, comprising two identical polypeptide chains comprising an amino acid sequence selected from SEQ ID NOs: 66, 67, 68, 69, 101-106 or at least 95% identical thereto.

44. The prodrug of any one of claims 1-34, comprising a first polypeptide chain and a second polypeptide chain which form a heterodimer, wherein
the first polypeptide chain comprises SEQ ID NO: 63 or 107, or an amino acid sequence at least 95% identical thereto, and
the second polypeptide chain comprises SEQ ID NO: 64, 65, 105, or 106, or an amino acid sequence at least 95% identical thereto.

45. The prodrug of any one of claims 1-34, comprising a first polypeptide chain and a second polypeptide chain which form a heterodimer; wherein
the first polypeptide chain comprises SEQ ID NO: 107 or an amino acid sequence at least 95% identical thereto, and
the second polypeptide chain comprises SEQ ID NO:105 or 106, or an amino acid sequence at least 95% identical thereto.

46. The prodrug of any one of claims 1-34, comprising two identical light chains and two identical heavy chains, wherein
the light chain comprises SEQ ID NO: 108 or an amino acid sequence at least 95% identical thereto SEQ ID NO: 108, and
the heavy chain comprises SEQ ID NO: 109, 110, 111, or 113, or an amino acid sequence at least 95% identical thereto.

47. The prodrug of any one of claims 1-34, comprising two identical light chains, a first heavy chain polypeptide chain, and a second heavy chain polypeptide chain, wherein
the light chain comprises SEQ ID NO: 108 or an amino acid sequence at least 95% identical thereto,
the first heavy chain polypeptide chain comprises SEQ ID NO: 110, or 111, or an amino acid sequence at least 95% identical thereto, and
the second heavy chain polypeptide chain comprises SEQ ID NO: 112, or an amino acid sequence at least 95% identical thereto.

48. The prodrug of any one of claims 1-34, comprising two identical light chains and two identical heavy chains, wherein
the light chain comprises SEQ ID NO: 116, or an amino acid sequence at least 95% identical thereto, and
the heavy chain polypeptide chain comprises SEQ ID NO: 114 or 115, or an amino acid sequence at least 95% identical thereto.

49. The prodrug of any one of the preceding claims, wherein the prodrug has a higher activity modulating an immune cell which expresses both the antigen targeted by the carrier moiety and a receptor for IL-10 or TGF-β than an immune cell which does not express both or either of the antigen and the cytokine receptor.

50. A pharmaceutical composition comprising the prodrug of any one of claims 1-49 and a pharmaceutically acceptable excipient.

51. A polynucleotide or polynucleotides encoding the prodrug of any one of claims 1-49.

52. An expression vector or vectors comprising the polynucleotide or polynucleotides of claim 51.

53. A host cell comprising the vector(s) of claim 52, optionally wherein the gene(s) encoding matriptase, uPA, MMP-2, MMP3, and/or MMP-9 are knocked out in the host cell.

54. A method of making the prodrug of any one of claims 1-49, comprising
culturing the host cell of claim 53 under conditions that allow expression of the prodrug, wherein the host cell is a mammalian cell, and
isolating the prodrug.

55. A method of treating an autoimmune disease or inflammatory condition in a patient in need thereof, comprising administering to the patient the prodrug of any one of claims 1-49.

56. A prodrug of any one of claims 1-49 for use in treating an autoimmune disease or inflammatory condition in a patient in need thereof.

57. Use of a prodrug of any one of claims 1-49 for the manufacture of a medicament for treating an autoimmune disease or inflammatory condition in a patient in need thereof.

58. The method of claim 55, the prodrug for use of claim 56, or the use of claim 57, wherein the prodrug is to be administered in combination with a pharmaceutical composition comprising an IL-2 mutein, an antagonist of TNFα, an antagonist of IL-12, an antagonist of IL-17 or its receptor, an antagonist of IL-23 or its receptor, an antagonist of IL-6 or its receptor, an antagonist of IL-5 or its receptor, an antagonist of IL-4 or its receptor, an antagonist of IL-1β or its receptor, an antagonist of interferon alpha receptor-1 (INFAR-1), an antagonist of CD40, an antagonist of CD80, or an antagonist of CD86.

59. The method, the prodrug for use, or the use of any one of claims 55-58, wherein the autoimmune disease or inflammation condition is selected from the group consisting of asthma, atopic dermatitis, Type I diabetes, diabetic ulcers, allergy, psoriasis, rheumatoid arthritis, multiple sclerosis, osteoarthritis, graft vs host disease (GvHD), lupus nephritis, systemic lupus erythematosus (SLE), Alzheimer's disease, a neuron degeneration disease, an inflammatory bowel disease, ulcerative colitis, Crohn's disease NASH, atherosclerosis, and systemic sclerosis.
